SEQUENCE LISTING (60 sequences)

SEQ ID NO: 1 - protein, 8 aa, Artificial Sequence
  synthetic
GRGDSPKC

SEQ ID NO: 2 - protein, 5 aa, Artificial Sequence
  synthetic
RGDFC

SEQ ID NO: 3 - protein, 29 aa, Artificial Sequence
  synthetic
AALEALAEALEALAEALEALAEAAAAGGC

SEQ ID NO: 4 - protein, 30 aa, Artificial Sequence
  synthetic
AALAEALAEALAEALAEALAEALAAAAGGC

SEQ ID NO: 5 - protein, 15 aa, Artificial Sequence
  synthetic
ALEALAEALEALAEA

SEQ ID NO: 6 - protein, 22 aa, Artificial Sequence
  synthetic
GLFEAIEGFIENGWEGMIWDYG

SEQ ID NO: 7 - protein, 23 aa, Artificial Sequence
  synthetic
GLFGAIAGFIENGWEGMIDGWYG

SEQ ID NO: 8 - protein, 48 aa, Artificial Sequence
  synthetic
GLFEAIEGFIENGWEGMIDGWYGCGLFEAIEGFIENGWEGMIDGWYGC

SEQ ID NO: 9 - protein, 44 aa, Artificial Sequence
  synthetic
GLFEAIEGFIENGWEGMIDGGCGLFEAIEGFIENGWEGMIDGGC

SEQ ID NO: 10 - protein, 35 aa, Artificial Sequence
  synthetic
GLFGALAEALAEALAEHLAEALAEALEALAAGGSC

SEQ ID NO: 11 - protein, 34 aa, Artificial Sequence
  synthetic
GLFEAIEGFIENGWEGLAEALAEALEALAAGGSC

SEQ ID NO: 12 - protein, 41 aa, Artificial Sequence
  synthetic
GLFEAIEGFIENGWEGNIDGKGLFEAIEGFIENGWEGNIDG

SEQ ID NO: 13 - protein, 19 aa, Artificial Sequence
  synthetic
LFEALLELLESLWELLLEA

SEQ ID NO: 14 - protein, 20 aa, Artificial Sequence
  synthetic
GLFKALLKLLKSLWKLLLKA

SEQ ID NO: 15 - protein, 20 aa, Artificial Sequence
  synthetic
GLFRALLRLLRSLWRLLLRA

SEQ ID NO: 16 - protein, 30 aa, Artificial Sequence
  synthetic
WEAKLAKALAKALAKHLAKALAKALKACEA

SEQ ID NO: 17 - protein, 22 aa, Artificial Sequence
  synthetic
GLFFEAIAEFIEGGWEGLIEGC

SEQ ID NO: 18 - protein, 26 aa, Artificial Sequence
  synthetic
GIGAVLKVLTTGLPALISWIKRKRQQ

SEQ ID NO: 19 - protein, 8 aa, Artificial Sequence
  synthetic
HHHHHWYG

SEQ ID NO: 20 - protein, 10 aa, Artificial Sequence
  synthetic
CHKKKKKKHC

SEQ ID NO: 21 - protein, 16 aa, Artificial Sequence
  synthetic
RQIKIWFQNRRMKWKK

SEQ ID NO: 22 - protein, 14 aa, Artificial Sequence
  synthetic
GRKKRRQRRRPPQC

SEQ ID NO: 23 - protein, 27 aa, Artificial Sequence
  synthetic
GALFLGWLGAAGSTMGAWSQPKKKRKV

SEQ ID NO: 24 - protein, 18 aa, Artificial Sequence
  synthetic
LLIILRRRIRKQAHAHSK

SEQ ID NO: 25 - protein, 26 aa, Artificial Sequence
  synthetic
GWTLNSAGYLLKINLKALAALAKKIL

SEQ ID NO: 26 - protein, 18 aa, Artificial Sequence
  synthetic
KLALKLALKALKAALKLA

SEQ ID NO: 27 - protein, 9 aa, Artificial Sequence
  synthetic
RRRRRRRRR

SEQ ID NO: 28 - protein, 10 aa, Artificial Sequence
  synthetic
KFFKFFKFFK

SEQ ID NO: 29 - protein, 37 aa, Artificial Sequence
  synthetic
LLGDFFRKSKEKIGKEFKRIVQRIKDFLRNLVPRTES

SEQ ID NO: 30 - protein, 31 aa, Artificial Sequence
  synthetic
SWLSKTAKKLENSAKKRISEGIAIAIQGGPR

SEQ ID NO: 31 - protein, 30 aa, Artificial Sequence
  synthetic
ACYCRIPACIAGERRYGTCIYQGRLWAFCC

SEQ ID NO: 32 - protein, 36 aa, Artificial Sequence
  synthetic
DHYNCVSSGGQCLYSACPIFTKIQGTCYRGKAKCCK

SEQ ID NO: 33 - protein, 42 aa, Artificial Sequence
  synthetic
RRRPRPPYLPRPRPPPFFPPRLPPRIPPGFPPRFPPRFPGKR

SEQ ID NO: 34 - protein, 13 aa, Artificial Sequence
  synthetic
ILPWKWPWWPWRR

SEQ ID NO: 35 - protein, 16 aa, Artificial Sequence
  synthetic
AAVALLPAVLLALLAP

SEQ ID NO: 36 - protein, 11 aa, Artificial Sequence
  synthetic
AALLPVLLAAP

SEQ ID NO: 37 - protein, 12 aa, Artificial Sequence
  synthetic
RKCRIVVIRVCR

SEQ ID NO: 38 - RNA, 21 nt, Artificial Sequence
  synthetic
aaaauccaaccuacaaguuca

SEQ ID NO: 39 - DNA, 23 bp, Artificial Sequence
  synthetic
ugaacutguagguuggauuuucg

SEQ ID NO: 40 - RNA, 23 nt, Artificial Sequence
  synthetic
cgaaaauccaaccuacaaguucu

SEQ ID NO: 41 - DNA, 23 bp, Artificial Sequence
  synthetic
ugaacutguagguuggauuuucg

SEQ ID NO: 42 - DNA, 23 bp, Artificial Sequence
  synthetic
ugaacutguagguuggauuuucg

SEQ ID NO: 43 - RNA, 21 nt, Artificial Sequence
  synthetic
aaaauccaaccuacaaguuca

SEQ ID NO: 44 - DNA, 23 bp, Artificial Sequence
  synthetic
ugaacutguagguuggauuuucg

SEQ ID NO: 45 - RNA, 21 nt, Artificial Sequence
  synthetic
ggcuacgaaaauccaaccuaa

SEQ ID NO: 46 - RNA, 21 nt, Artificial Sequence
  synthetic
aaagagcaaaacuauucagaa

SEQ ID NO: 47 - RNA, 21 nt, Artificial Sequence
  synthetic
aaagagcaaaacuauucagaa

SEQ ID NO: 48 - RNA, 21 nt, Artificial Sequence
  synthetic
uuuaugauuuacucauuauca

SEQ ID NO: 49 - RNA, 21 nt, Artificial Sequence
  synthetic
ggcuacgaaaauccaaccuaa

SEQ ID NO: 50 - RNA, 21 nt, Artificial Sequence
  synthetic
aaagagcaaaacuauucagaa

SEQ ID NO: 51 - RNA, 21 nt, Artificial Sequence
  synthetic
aaagagcaaaacuauucagaa

SEQ ID NO: 52 - RNA, 21 nt, Artificial Sequence
  synthetic
uuuaugauuuacucauuauca

SEQ ID NO: 53 - DNA, 23 bp, Artificial Sequence
  synthetic
uuaggutggautuucguagccgu

SEQ ID NO: 54 - DNA, 23 bp, Artificial Sequence
  synthetic
uucugaauagutuugcucuuucu

SEQ ID NO: 55 - DNA, 23 bp, Artificial Sequence
  synthetic
uucugaauagutuugcucuuucu

SEQ ID NO: 56 - RNA, 23 nt, Artificial Sequence
  synthetic
ugauaaugaguaaaucauaaaug

SEQ ID NO: 57 - DNA, 23 bp, Artificial Sequence
  synthetic
uuaggutggautuucguagccgu

SEQ ID NO: 58 - DNA, 23 bp, Artificial Sequence
  synthetic
uucugaauagutuugcucuuucu

SEQ ID NO: 59 - DNA, 23 bp, Artificial Sequence
  synthetic
uucugaauagutuugcucuuucu

SEQ ID NO: 60 - RNA, 23 nt, Artificial Sequence
  synthetic
ugauaaugaguaaaucauaaaug

(19) United States
(12) Patent Application Publication
(10) Pub. No.: US 2024/0175028 A1
(43) Pub. Date: May 30, 2024

(54) DOSING OF siRNA COMPOUNDS TO THE CISTERNA MAGNA

(71) Applicant: ALNYLAM PHARMACEUTICALS, INC, Cambridge, MA (US)

(72) Inventors: Tanya Z. Fischer, Newton, MA (US); Bret L. Bostwick, Cambridge, MA (US); Christopher S. Thiele, Melrose, MA (US); Charalambos Kaittanis, Cambridge, MA (US); Jeffrey C. Kurz, Acton, MA (US); Jeffrey W. Allen, Cambridge, MA (US)

(73) Assignee: ALNYLAM PHARMACEUTICALS, INC, Cambridge, MA (US)

(21) Appl. No.: 18/548,634

(22) PCT Filed: Mar. 4, 2022
(86) PCT No.: PCT/US2022/018902

§ 371 Date: Sep. 1, 2023

Related U.S. Application Data
(60) Provisional application No. 63/157,474, filed on Mar. 5, 2021.
(60) Provisional application No. 63/214,716, filed on Jun. 24, 2021.

(51) Int. Cl. C12N 15/113; A61K 9/00; A61K 47/54

(52) U.S. Cl. CPC C12N 15/113; A61K 9/0019; A61K 9/0085; A61K 47/543; C12N 2310/14

(57) ABSTRACT

One aspect of the present invention relates to a double stranded iRNA agent comprising an antisense strand which is complementary to a target gene and one or more lipophilic moieties conjugated to one or more internal positions on at least one strand, which provides for targeting to, and uptake by, tissues and cells of the CNS via administration to a subarachnoid space, e.g., the cisterna magna. Another aspect of the invention relates to a method of gene silencing in tissues and cells of the CNS, that includes administering to the subarachnoid space of a subject in need thereof a therapeutically effective amount of the lipophilic moieties-conjugated double-stranded iRNAs.

### CROSS-REFERENCE TO RELATED APPLICATIONS

The present application is related to and claims priority under 35 U.S.C. § 119(e) to U.S. Provisional Patent Application No. 63/157,474, entitled “DOSING OF siRNA COMPOUNDS TO THE CISTERNA MAGNA,” filed Mar. 5, 2021, and U.S. Provisional Patent Application No. 63/214,716, entitled “DOSING OF siRNA COMPOUNDS TO THE CISTERNA MAGNA,” filed Jun. 24, 2021. The entire contents of the aforementioned patent applications are incorporated herein by this reference.

### FIELD

The disclosure relates to a double stranded iRNA agent comprising one or more lipophilic moieties conjugated to one or more internal positions on at least one strand, optionally via a linker or carrier, which provides for targeting to, and uptake by, tissues and cells of the CNS via administration to a subarachnoid space, e.g., the cisterna magna.

### SEQUENCE LISTING

The instant application contains a Sequence Listing which has been filed electronically in ASCII format and is hereby incorporated by reference in its entirety. Said ASCII copy, created on Mar. 4, 2022, is named BN00005_0182_SL_ST25.txt and is 16 KB in size.

### BACKGROUND

Efficient delivery of an iRNA agent to cells in vivo requires specific targeting, efficient uptake, and substantial protection from the extracellular environment, particularly serum proteins, immune cells or components of biological matrices. RNAi-based therapeutics show promising clinical data for treatment of liver-associated disorders. However, siRNA delivery into extra-hepatic tissues remains an obstacle, limiting the use of siRNA-based therapies.

Similar to other therapeutic modalities, delivery of oligonucleotides to the central nervous system (CNS) poses particular problems due to the blood brain barrier (BBB). Free oligonucleotides either cannot cross the BBB or their crossing might be minimal. One means to deliver oligonucleotides into the CNS is by intrathecal delivery into the cerebrospinal fluid (CSF), the most common means of which is by dural puncture between two lumbar vertebrae (henceforth referred to as “lumbar puncture”). Broad distribution to all regions of the CNS depends on CSF flow and diffusion rostrally along the spinal cord up to and around the brain. Furthermore, the oligonucleotides need also to be efficiently internalized into target cells of the CNS to achieve the desired therapeutic effect. Unfortunately, rapid turnover of the CSF (˜4 hours), as well as constant fluid flows, may limit compound uptake by CNS tissues and cells after intrathecal dosing.

Thus, there is a continuing need for new and improved methods for delivering siRNA molecules in vivo in the CNS to achieve and enhance the therapeutic potential of iRNA agents for treating indications whose treatment depends on access of the therapeutic agent into the brain.

### SUMMARY

An alternative to lumbar puncture for intrathecal administration is intracisternal dosing, where the RNAi is dosed by dural puncture directly into the cisterna magna. Intracisternal injection maximizes RNAi concentrations in proximity to the brain, avoiding the dilution that accompanies rostral transit from the lumbar spine, and potential clearance through CSF drainage pathways along the spine. Intracisternal administration maximizes the time that RNAi is in contact with the brain at concentrations sufficient to drive uptake into brain tissue, providing improved exposure that may translate to better cellular access, tissue pharmacokinetics and siRNA activity, leading to improved therapeutic outcomes.

The instant disclosure is based, at least in part, upon the unexpected discovery that administration of therapeutic oligonucleotides to the cisterna magna is capable of producing specific and efficient knockdown of targeted mRNA(s) and associated protein product(s) within multiple CNS tissues and cells.

One aspect of the invention provides a method of reducing the expression of a target gene in a central nervous system (CNS) tissue or cell of a subject via administration of a double-stranded iRNA agent to a subarachnoid space of the subject, the method involving contacting the tissue or cell with a double-stranded iRNA agent having: an antisense strand which comprises a region of complementarity to the target gene which comprises at least 15 contiguous nucleotides differing by no more than 3 nucleotides from any one of the antisense sequences listed in Table 2, Table 3 or Table 8; a sense strand which is complementary to the antisense strand; and one or more lipophilic moieties conjugated to one or more internal positions on at least one strand, optionally via a linker or carrier.

In some embodiments, the lipophilicity of the lipophilic moiety, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the double-stranded iRNA agent, measured by the unbound fraction in the plasma protein binding assay of the double-stranded iRNA agent, exceeds 0.2. In one embodiment, the plasma protein binding assay determined is an electrophoretic mobility shift assay (EMSA) using human serum albumin protein. The hydrophobicity of the double-stranded iRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. Exemplary lipophilic moieties are lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

Suitable lipophilic moieties also include those containing a saturated or unsaturated C₄-C₃₀ hydrocarbon chain (e.g., C₄-C₃₀ alkyl or alkenyl), and an optional functional group selected from the group consisting of hydroxyl, amine, carboxylic acid, sulfonate, phosphate, thiol, azide, and alkyne. The functional groups are useful to attach the lipophilic moiety to the iRNA agent. In some embodiments, the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

In embodiments, the method involves injecting the double-stranded iRNA agent into cerebrospinal fluid (CSF) of the subarachnoid space of the subject. Optionally, the subarachnoid space of the subject is or includes the cisterna magna.

The lipophilic moiety may be conjugated to the iRNA agent via a direct attachment to the ribosugar of the iRNA agent. Alternatively, the lipophilic moiety may be conjugated to the iRNA agent via a linker or a carrier.

In certain embodiments, the lipophilic moiety are conjugated to the iRNA agent via one or more linkers (tethers).

In some embodiments, the lipophilic moiety is conjugated to the double-stranded iRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the lipophilic moiety is conjugated to the double-stranded iRNA agent via a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

In one embodiment, the lipophilic moiety is conjugated to one or more internal positions on at least one strand, which include all positions except the terminal two positions from each end of the strand. In one embodiment, the lipophilic moiety is conjugated to one or more internal positions on at least one strand, which include all positions except the terminal three positions from each end of the strand.

In one embodiment, the lipophilic moiety is conjugated to one or more internal positions on at least one strand, which exclude the cleavage site region of the sense strand. For instance, the internal positions exclude positions 9-12 counting from the 5′-end of the sense strand. Alternatively, the internal positions exclude positions 11-13 counting from the 3′-end of the sense strand.

In one embodiment, the lipophilic moiety is conjugated to one or more internal positions on at least one strand, which exclude the cleavage site region of the antisense strand. For instance, the internal positions exclude positions 12-14 counting from the 5′-end of the antisense strand.

In one embodiment, the lipophilic moiety is conjugated to one or more internal positions on at least one strand, which exclude positions 11-13 on the sense strand, counting from the 3′-end, and positions 12-14 on the antisense strand, counting from the 5′-end.

In one embodiment, one or more lipophilic moieties are conjugated to one or more of the following internal positions: positions 4-8 and 13-18 on the sense strand, and positions 6-10 and 15-18 on the antisense strand, counting from the 5′end of each strand.

In one embodiment, one or more lipophilic moieties are conjugated to one or more of the following internal positions: positions 5, 6, 7, 15, and 17 on the sense strand, and positions 15 and 17 on the antisense strand, counting from the 5′end of each strand.

In some embodiments, the sense and antisense strands of the double-stranded iRNA agent are each 15 to 30 nucleotides in length.

In one embodiment, the sense and antisense strands of a double-stranded iRNA agent are each 19 to 25 nucleotides in length.

In one embodiment, the sense and antisense strands of the double-stranded iRNA agent are each 21 to 23 nucleotides in length.

In some embodiments, the double-stranded iRNA agent comprises a single-stranded overhang on at least one of the termini. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length.

In one embodiment, the sense strand of the double-stranded iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, the lipophilic moiety is conjugated to a nucleobase, sugar moiety, or internucleosidic linkage of the double-stranded iRNA agent.

In some embodiments, the double-stranded iRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the double-stranded iRNA agent does not contain a 5′-vinyl phosphonate (VP).

In some embodiments, the double-stranded iRNA agent further comprises a targeting ligand that targets a receptor which mediates delivery to a specific CNS tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, and LDL receptor ligand.

Another aspect of the invention relates to a method of reducing the expression of a target gene in a subject, the method involving administering to the cisterna magna of the subject a double-stranded iRNA agent possessing an antisense strand which is complementary to a target gene; a sense strand which is complementary to said antisense strand; and one or more lipophilic moieties conjugated to one or more internal positions on at least one strand, optionally via a linker or carrier.

In some embodiments, the double-stranded iRNA agent is administered to the cisterna magna via intracisternal magna (ICM) injection. By ICM administration of the double-stranded iRNA agent, the method can reduce the expression of a target gene in a brain or spine tissue, for instance, cerebral cortex (e.g., frontal, temporal, parietal, and/or occipital cortex), striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG).

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25.

In some embodiments, the expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the expression of the target gene is reduced by about 20%, about 25%, about 30%, about 35%, about 40%, about 45%, about 50%, about 55%, about 60%, about 65%, about 70%, or about 75% within about 29 days.

In some embodiments, the expression of the target gene is reduced by about 25% to about 35% within about 29 days.

In some embodiments, the expression of the target gene is reduced by about 35% to about 45% within about 29 days.

In some embodiments, the expression of the target gene is reduced by about 45% to about 55% within about 29 days.

In some embodiments, the expression of the target gene is reduced by about 25% to about 50% within about 29 days.

In some embodiments, the expression of the target gene is reduced by about 35% to about 50% within about 29 days.

In some embodiments, the expression of the target gene is reduced by about 45% to about 50% within about 29 days.

In some embodiments, the expression of the target gene is reduced by about 50% within about 29 days.

In some embodiments, the expression of the target gene is reduced by about 25% to about 50% within about 25 to about 31 days.

In some embodiments, the expression of the target gene is reduced by about 35% to about 50% within about 25 to about 31 days.

In some embodiments, the expression of the target gene is reduced by about 45% to about 50% within about 25 to about 31 days.

In some embodiments, the expression of the target gene is reduced by about 50% within about 25 to about 31 days.

In some embodiments, the double-stranded iRNA agent is detected in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell.

In some embodiments, the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected within about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected within about 25 days to about 30 days.

In some embodiments, the double-stranded iRNA agent is detected within about 29 days.

In some embodiments, the double-stranded iRNA agent is detected after at least 29 days.

In some embodiments, the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected after about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected after about 25 days to about 30 days.

In some embodiments, the double-stranded iRNA agent is detected after about 29 days.

In some embodiments, the expression of the target gene is reduced by at least 96%, at least 97%, at least 98%, at least 99%, or at least 100%.

In some embodiments, the expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

In embodiments, the sense strand comprises at least 15 contiguous nucleotides differing by no more than 3 nucleotides from the nucleotide sequence of the sense strand nucleotide sequence of duplex AD-454844, AD-1395836, AD-1397045, AD-961583, AD-961584, AD-961585, or AD-961586.

Another aspect of the invention relates to a method of treating a subject having a CNS disorder, comprising administering to the subject a therapeutically effective amount of a double-stranded RNAi agent, thereby treating the subject. The double-stranded RNAi agent comprises an antisense strand which comprises a region of complementarity to the target gene which comprises at least 15 contiguous nucleotides differing by no more than 3 nucleotides from any one of the antisense sequences listed in Table 2, Table 3, or Table 8; a sense strand which is complementary to said antisense strand; and one or more lipophilic moieties conjugated to one or more internal positions on at least one strand, optionally via a linker or carrier.

All the above embodiments relating to the lipophilic moieties and their conjugation to the double-stranded iRNA agent in the first aspect of the invention relating to the double-stranded iRNA agent are suitable in this aspect of the invention relating to a method of treating a subject having a CNS disorder. Exemplary CNS disorders that can be treated by the method of the invention include Alzheimer's disease, amyotrophic lateral sclerosis (ALS), frontotemporal dementia, Huntington's disease, Parkinson's disease, spinocerebellar ataxia, prion disorders, and lafora. In addition to traditional CNS disorders, this method is also suitable for treating diseases for which successful treatment requires the siRNA to access the brain. Exemplary disorders of this type may include obesity or metabolic syndromes which can be modified through modulation of the sympathetic neuroadipose axis, hypothalamus or pituitary gland.

Another aspect of the invention relates to a method of reducing the expression of a target gene in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell tissue or cell of a subject, that includes the steps of: contacting the tissue or cell of the subject via subarachnoid space administration of a double-stranded iRNA agent, where the agent includes an antisense strand complementary to the target gene; a sense strand complementary to the antisense strand; and one or more lipophilic moieties conjugated to one or more internal positions on at least one strand, optionally via a linker or carrier.

In some embodiments, the lipophilicity of the lipophilic moiety, measured by log K_{ow}, exceeds 0.

In some embodiments, the hydrophobicity of the double-stranded iRNA agent, measured by the unbound fraction in the plasma protein binding assay of the double-stranded iRNA agent, exceeds 0.2.

In some embodiments, the plasma protein binding assay is an electrophoretic mobility shift assay using human serum albumin protein.

In some embodiments, the lipophilic moiety contains a saturated or unsaturated C₁₆ hydrocarbon chain. In some embodiments, the lipophilic moiety is a saturated or unsaturated C₁₆ hydrocarbon chain. In some embodiments, the lipophilic moiety is a saturated C₁₆ hydrocarbon chain (e.g., n-hexadecyl).

In some embodiments, the step of contacting includes injecting the double-stranded iRNA agent into cerebrospinal fluid (CSF) of the subarachnoid space.

In some embodiments, the subarachnoid space is or includes the cisterna magna.

In some embodiments, the target gene is selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25. In some embodiments, the target gene is not HTT.

In some embodiments, the expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the expression of the target gene is reduced by about 20%, about 25%, about 30%, about 35%, about 40%, about 45%, about 50%, about 55%, about 60%, about 65%, about 70%, or about 75% within about 29 days.

In some embodiments, the expression of the target gene is reduced by about 25% to about 35% within about 29 days.

In some embodiments, the expression of the target gene is reduced by about 35% to about 45% within about 29 days.

In some embodiments, the expression of the target gene is reduced by about 45% to about 55% within about 29 days.

In some embodiments, the expression of the target gene is reduced by about 25% to about 50% within about 29 days.

In some embodiments, the expression of the target gene is reduced by about 35% to about 50% within about 29 days.

In some embodiments, the expression of the target gene is reduced by about 45% to about 50% within about 29 days.

In some embodiments, the expression of the target gene is reduced by about 50% within about 29 days.

In some embodiments, the expression of the target gene is reduced by about 25% to about 50% within about 25 to about 31 days.

In some embodiments, the expression of the target gene is reduced by about 35% to about 50% within about 25 to about 31 days.

In some embodiments, the expression of the target gene is reduced by about 45% to about 50% within about 25 to about 31 days.

In some embodiments, the expression of the target gene is reduced by about 50% within about 25 to about 31 days.

In some embodiments, the double-stranded iRNA agent is detected in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell tissues or cells.

In some embodiments, the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected within about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected within about 25 days to about 30 days.

In some embodiments, the double-stranded iRNA agent is detected within about 29 days.

In some embodiments, the double-stranded iRNA agent is detected after at least 29 days.

In some embodiments, the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected after about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected after about 25 days to about 30 days.

In some embodiments, the double-stranded iRNA agent is detected after about 29 days.

In some embodiments, the expression of the target gene is reduced by at least 96%, at least 97%, at least 98%, at least 99%, or at least 100%.

In some embodiments, the expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

Another aspect of the invention relates to a method of reducing the expression of a target gene in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell of a subject, the method including the steps of: administering to the subject a double-stranded iRNA agent that includes an antisense strand complementary to the target gene, a sense strand complementary to the antisense strand; and one or more lipophilic moieties conjugated to one or more internal positions on at least one strand, optionally via a linker or carrier.

In some embodiments, the step of administering includes injecting the double-stranded iRNA agent into cerebrospinal fluid (CSF) of a subarachnoid space.

In some embodiments, the subarachnoid space is or includes the cisterna magna.

In some embodiments, the step of administering further includes injecting the double-stranded iRNA agent into the cisterna magna of the subject.

In some embodiments, the method reduces the expression of the target gene in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell of the subject.

In some embodiments, the target gene is selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25. In some embodiments, the target gene is not HTT.

In some embodiments, the expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the expression of the target gene is reduced by about 20%, about 25%, about 30%, about 35%, about 40%, about 45%, about 50%, about 55%, about 60%, about 65%, about 70%, or about 75% within about 29 days.

In some embodiments, the expression of the target gene is reduced by about 25% to about 35% within about 29 days.

In some embodiments, the expression of the target gene is reduced by about 35% to about 45% within about 29 days.

In some embodiments, the expression of the target gene is reduced by about 45% to about 55% within about 29 days.

In some embodiments, the expression of the target gene is reduced by about 25% to about 50% within about 29 days.

In some embodiments, the expression of the target gene is reduced by about 35% to about 50% within about 29 days.

In some embodiments, the expression of the target gene is reduced by about 45% to about 50% within about 29 days.

In some embodiments, the expression of the target gene is reduced by about 50% within about 29 days.

In some embodiments, the expression of the target gene is reduced by about 25% to about 50% within about 25 to about 31 days.

In some embodiments, the expression of the target gene is reduced by about 35% to about 50% within about 25 to about 31 days.

In some embodiments, the expression of the target gene is reduced by about 45% to about 50% within about 25 to about 31 days.

In some embodiments, the expression of the target gene is reduced by about 50% within about 25 to about 31 days.

In some embodiments, the double-stranded iRNA agent is detected in in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell tissues or cells.

In some embodiments, the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected within about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected within about 25 days to about 30 days.

In some embodiments, the double-stranded iRNA agent is detected within about 29 days.

In some embodiments, the double-stranded iRNA agent is detected after at least 29 days.

In some embodiments, the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected after about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected after about 25 days to about 30 days.

In some embodiments, the double-stranded iRNA agent is detected after about 29 days.

In some embodiments, the expression of the target gene is reduced by at least 96%, at least 97%, at least 98%, at least 99%, or at least 100%.

In some embodiments, the expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

In some embodiments, the double-stranded iRNA agent is administered intravitreally.

In embodiments, the sense strand comprises at least 15 contiguous nucleotides differing by no more than 3 nucleotides from the nucleotide sequence of the sense strand nucleotide sequence of duplex AD-454844, AD-1395836, AD-1397045, AD-961583, AD-961584, AD-961585, or AD-961586.

Another aspect of the invention relates to a method of treating a subject having a CNS disorder, including the step of: administering to a subarachnoid space of the subject a therapeutically effective amount of a double-stranded RNAi agent, thereby treating the subject.

In some embodiments, the double-stranded iRNA agent includes an antisense strand which comprises a region of complementarity to the target gene which comprises at least 15 contiguous nucleotides differing by no more than 3 nucleotides from any one of the antisense sequences listed in Table 2 or Table 3, a sense strand complementary to the antisense strand, and one or more lipophilic moieties conjugated to one or more internal positions on at least one strand, optionally via a linker or carrier.

In some embodiments, the hydrophobicity of the double-stranded iRNA agent, measured by the unbound fraction in the plasma protein binding assay of the double-stranded iRNA agent, exceeds 0.2, or the lipophilicity of the lipophilic moiety, measured by log K_{ow}, exceeds 0.

In some embodiments, the plasma protein binding assay is an electrophoretic mobility shift assay using human serum albumin protein.

In some embodiments, the lipophilic moiety contains a saturated or unsaturated C₁₆ hydrocarbon chain.

In some embodiments, the CNS disorder is selected from the group consisting of Alzheimer's disease (AD), amyotrophic lateral sclerosis (ALS), frontotemporal dementia, Huntington's disease (e.g., Huntington's chorea), Parkinson's disease, spinocerebellar disease, prion disease, and lafora disease.

In some embodiments, the CNS disorder is a disorder affecting the striatum.

In some embodiments, administering further includes a step of injecting the double-stranded iRNA agent into cerebrospinal fluid (CSF) of the subarachnoid space. Optionally, the subarachnoid space is or includes the cisterna magna.

In some embodiments, administering further includes a step of injecting the double-stranded iRNA agent into the cisterna magna of the subject.

In some embodiments, the method reduces the expression of the target gene in brain tissue surrounding the striatum of the subject.

In some embodiments, the double-stranded iRNA agent is directed to a target gene selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25.

In some embodiments, the target gene is not HTT.

In some embodiments, the expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

In some embodiments, the expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the expression of the target gene is reduced by about 20%, about 25%, about 30%, about 35%, about 40%, about 45%, about 50%, about 55%, about 60%, about 65%, about 70%, or about 75% within about 29 days.

In some embodiments, the expression of the target gene is reduced by about 25% to about 35% within about 29 days.

In some embodiments, the expression of the target gene is reduced by about 35% to about 45% within about 29 days.

In some embodiments, the expression of the target gene is reduced by about 45% to about 55% within about 29 days.

In some embodiments, the expression of the target gene is reduced by about 25% to about 50% within about 29 days.

In some embodiments, the expression of the target gene is reduced by about 35% to about 50% within about 29 days.

In some embodiments, the expression of the target gene is reduced by about 45% to about 50% within about 29 days.

In some embodiments, the expression of the target gene is reduced by about 50% within about 29 days.

In some embodiments, the expression of the target gene is reduced by about 25% to about 50% within about 25 to about 31 days.

In some embodiments, the expression of the target gene is reduced by about 35% to about 50% within about 25 to about 31 days.

In some embodiments, the expression of the target gene is reduced by about 45% to about 50% within about 25 to about 31 days.

In some embodiments, the expression of the target gene is reduced by about 50% within about 25 to about 31 days.

In some embodiments, the double-stranded iRNA agent is detected in a cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG) tissue or cell tissues or cells.

In some embodiments, the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected within about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected within about 25 days to about 30 days.

In some embodiments, the double-stranded iRNA agent is detected within about 29 days.

In some embodiments, the double-stranded iRNA agent is detected after at least 29 days.

In some embodiments, the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

In some embodiments, the double-stranded iRNA agent is detected after about 1 to about 5 days, about 5 to about 10 days, about 10 to about 15 days, about 15 to about 20 days, about 20 to about 25 days, or about 25 to about 30 days.

In some embodiments, the double-stranded iRNA agent is detected after about 25 days to about 30 days.

In some embodiments, the double-stranded iRNA agent is detected after about 29 days.

In some embodiments, the expression of the target gene is reduced by at least 96%, at least 97%, at least 98%, at least 99%, or at least 100%.

In some embodiments, the expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

In embodiments, the sense strand comprises at least 15 contiguous nucleotides differing by no more than 3 nucleotides from the nucleotide sequence of the sense strand nucleotide sequence of duplex AD-454844, AD-1395836, AD-1397045, AD-961583, AD-961584, AD-961585, or AD-961586.

In another embodiment, the RNAi agent is a pharmaceutically acceptable salt thereof. “Pharmaceutically acceptable salts” of each of RNAi agents herein include, but are not limited to, a sodium salt, a calcium salt, a lithium salt, a potassium salt, an ammonium salt, a magnesium salt, an mixtures thereof. One skilled in the art will appreciate that the RNAi agent, when provided as a polycationic salt having one cation per free acid group of the optionally modified phosphodiester backbone and/or any other acidic modifications (e.g., 5′-terminal phosphonate groups). For example, an oligonucleotide of “n” nucleotides in length contains n−1 optionally modified phosphodiesters, so that an oligonucleotide of 21 nt in length may be provided as a salt having up to 20 cations (e.g., 20 sodium cations). Similarly, an RNAi agents having a sense strand of 21 nt in length and an antisense strand of 23 nt in length may be provided as a salt having up to 42 cations (e.g., 42 sodium cations). In the preceding example, where the RNAi agent also includes a 5′-terminal phosphate or a 5′-terminal vinylphosphonate group, the RNAi agent may be provided as a salt having up to 44 cations (e.g., 44 sodium cations).

### BRIEF DESCRIPTION OF THE DRAWINGS

FIG. 1 is a scheme showing ligands, such as lipophilic moieties, that are conjugated to siRNAs at internal positions of the sense or antisense strand (i.e., somewhere within the siRNA sequence).

FIG. 2 is a scheme showing ligands, such as lipophilic moieties, that are conjugated to siRNAs through linkers or carriers at the 3′- and/or 5′-ends of the sense or antisense strand.

FIG. 3 is a scheme showing ligands, such as lipophilic moieties, that are conjugated to siRNAs via bio-cleavable linkers.

FIG. 4A is a graph demonstrating APP knockdown by subject (0101, 0102, 0103, and 0104) by AD-454844 versus aCSF control in the lumbar spine, cervical spine, prefrontal cortex, hypothalamus, and striatum following intracisternal magna (ICM) administration.

FIG. 4B is a graph demonstrating that ICM dosing of AD-454844 results in sufficient siRNA delivery throughout the spine and brain, including lumbar spine, cervical spine, prefrontal cortex, hypothalamus, to produce APP mRNA knockdown at the tissue level as measured by both APPα and APPβ.

FIG. 4C is a picture of the AD-454844 siRNA administered by ICM.

FIG. 5 is a schematic images of APP-targeted RNAi agents AD-961583, AD-961584, AD-961585, and AD-961586, as described in Example X.

FIG. 6A is a schematic showing a 2′-aminohexyl-modified C16-siRNA.

FIG. 6B is a diagram showing ¹²⁴I-SIB conjugation.

FIG. 7 is a schematic showing non-human primate (NIP) study groups and ¹²⁴I-SIB-siRNA dose formulations.

FIG. 8 is a graph showing SIB-siRNA radiolabeling.

FIG. 9 is a diagram showing 2′-aminohexyl-modified AD-454844 duplex (AD-1395836).

FIG. 10 is a positron emission tomography (PET) image showing uptake of ¹²⁴I-SIB-siRNA in the cranial cavity of a NHP subject.

FIG. 11A is a series of time course images of a NHP subject injected with ¹²⁴I-SIB-siRNA.

FIG. 11B is a series of time course images of a NHP subject injected with ¹²⁴I-SIB-siRNA.

FIG. 11C is a series of time course images of a NHP subject injected with ¹²⁴I-SIB-siRNA.

FIG. 11D is a series of time course images of a NHP subject injected with ¹²⁴I-SIB-siRNA.

FIG. 11E is a series of time course images of a NHP subject injected with ¹²⁴I-SIB-siRNA.

FIG. 11F is a series of time course images of a NHP subject injected with ¹²⁴I-SIB-siRNA.

FIG. 12A is an image of a representative NHP brain imaged two weeks after dosing with ¹²⁴I-SIB-siRNA.

FIG. 12B is an image of a representative NHP brain imaged two weeks after dosing with ¹²⁴I-SIB-siRNA.

FIG. 13 is a graph showing the percent of mRNA remaining relative to PBS on the y-axis vs. concentration of siRNA (nM) on the x-axis.

FIG. 14A is a representative CT image showing the systemic regions segmented for image analysis.

FIG. 14B is a representative CT image showing all brain subregions, except for the meninges, segmented for image analysis.

FIG. 14C is a representative CT image showing the meninges region segmented for image analysis, and including a coronal plane image, a sagittal plane image, and a transverse plane image.

FIG. 15A is a graph plotting PET (standardized uptake values, “SUV”) on the y-axis versus gamma counting (SUV) on the x-axis in selected peripheral organs and the CNS (listed to right of graph).

FIG. 15B is a graph plotting PET (SUV) on the y-axis versus gamma counting (SUV) on the x-axis in indicated spinal cord and brainstem regions (listed to right of graph).

FIG. 15C is a graph plotting PET (SUV) on the y-axis versus gamma counting (SUV) on the x-axis in indicated brain regions (listed to right of graph).

FIG. 16A is a bar graph of ex vivo standardized uptake values (SUV) obtained using gamma counting, with values shown on the y-axis, across eight indicated tissues (temporal cortex, prefrontal cortex, hypothalamus, brain stem, cerebellum, thalamus, hippocampus, and striatum) following either ICM (circles) or IT (diamonds) delivery, as assessed on Day 14 post-delivery. The inset shows gamma counting standardized uptake values (SUV) obtained in the kidneys and liver, as a control.

FIG. 16B is a bar graph of in vivo standardized uptake values (SUV) obtained using PET, with values shown on the y-axis, across nine indicated tissues (temporal cortex, prefrontal cortex, hypothalamus, brain stem, cerebellum, thalamus, hippocampus, striatum, and ventricle) following either ICM (circles) or IT (diamonds) delivery, as assessed on Day 14 post-delivery. The inset shows PET standardized uptake values (SUV) obtained in the kidneys and liver, as a control.

FIG. 17A is a bar graph showing in vivo PET standardized uptake values (SUV) on the y-axis, obtained in the indicated nine tissues (temporal cortex, prefrontal cortex, hypothalamus, brain stem, cerebellum, thalamus, hippocampus, striatum, and ventricle) shown on the x-axis, following either ICM (circles) or IT (diamonds) delivery, as assessed on Day 1 post-delivery.

FIG. 17B is a bar graph showing in vivo PET standardized uptake values (SUV) on the y-axis, obtained in the indicated nine tissues (temporal cortex, prefrontal cortex, hypothalamus, brain stem, cerebellum, thalamus, hippocampus, striatum, and ventricle) shown on the x-axis, following either ICM (circles) or IT (diamonds) delivery, as assessed on Day 2 post-delivery.

FIG. 17C is a bar graph showing in vivo PET standardized uptake values (SUV) on the y-axis, obtained in the indicated nine tissues (temporal cortex, prefrontal cortex, hypothalamus, brain stem, cerebellum, thalamus, hippocampus, striatum, and ventricle) shown on the x-axis, following either ICM (circles) or IT (diamonds) delivery, as assessed on Day 4 post-delivery.

FIG. 17D is a bar graph showing in vivo PET standardized uptake values (SUV) on the y-axis, obtained in the indicated nine tissues (temporal cortex, prefrontal cortex, hypothalamus, brain stem, cerebellum, thalamus, hippocampus, striatum, and ventricle) shown on the x-axis, following either ICM (circles) or IT (diamonds) delivery, as assessed on Day 7 post-delivery.

FIG. 17E is a bar graph showing in vivo PET standardized uptake values (SUV) on the y-axis, obtained in the indicated nine tissues (temporal cortex, prefrontal cortex, hypothalamus, brain stem, cerebellum, thalamus, hippocampus, striatum, and ventricle) shown on the x-axis, following either ICM (circles) or IT (diamonds) delivery, as assessed on Day 14 post-delivery.

FIG. 17F shows comparative bar graphs showing in vivo PET standardized uptake values (SUV) on the y-axes, obtained in the indicated nine tissues (temporal cortex, prefrontal cortex, hypothalamus, brain stem, cerebellum, thalamus, hippocampus, striatum, and ventricle) of the x-axis following either ICM (circles) or IT (diamonds) delivery, as assessed on Day 1 post-delivery (top graph) and on Day 14 post-delivery (bottom graph). Measured uptake of siRNA into non-CNS control tissues (i.e., kidney and liver) is shown separately to the right of each bar graph.

FIG. 18A is a bar graph showing ex vivo gamma counter standardized uptake values (SUV) on the y-axis in the three indicated spinal cord (SC) tissues (lumbar, thoracic, and cervical) of the x-axis following either ICM (circles) or IT (diamonds) delivery, as assessed on Day 14 post-delivery. In this figure, SC refers to spinal cord only.

FIG. 18B is a bar graph showing in vivo PET standardized uptake values (SUV) on the y-axis in indicated lumbar spinal cord (SC), lower thoracic SC, upper thoracic SC, cervical SC, lumbar spine, lower thoracic spine, upper thoracic spine, and cervical spine locations of the x-axis following either ICM (circles) or IT (diamonds) delivery, as assessed on Day 14 post-delivery. In this figure, SC refers to spinal cord and CSF, while spine refers to vertebrae.

FIG. 19 shows comparative bar graphs showing in vivo PET standardized uptake values (SUV) on the y-axis in indicated lumbar spinal cord (SC), lower thoracic SC, upper thoracic SC, cervical SC, lumbar spine, lower thoracic spine, upper thoracic spine, and cervical spine locations of the x-axis following either ICM (circles) or IT (diamonds) delivery, as assessed on Day 1 post-delivery (left graph) and on Day 14 post-delivery (right graph). SC refers to spinal cord and CSF, while spine refers to vertebrae.

FIG. 20A is a graph depicting a longitudinal overview of ICM delivery and IT delivery of siRNA in the brain, liver, and kidneys of non-human primates (NHPs), with PET data collected at Day 1, Day 2, Day 4, Day 7, and Day 14 shown on the x-axis and % ID shown on the y-axis. Data key is shown to the right of the graph. Brain refers to whole brain Region of Interest (ROI).

FIG. 20B is a dot plot of NHP PET data vs. rat SPECT data at Day 2 post-delivery. Tissue (brain ROI, live, and kidney) is shown on the x-axis and % ID is shown on the y-axis. Black circles (left dots of each pair) represent IT delivery in the rat, while purple circles (right dots of each pair) represent IT delivery in NHPs. The asterisk indicates that a statistically significant difference was observed between rat brain uptake and NHP brain uptake, whereas any differences between rat and NHP in liver and kidneys were not determined to be statistically significant.

FIG. 21A is a graph depicting percent of dose identified (% ID) by PET of whole brain NHP subjects, in the meninges following ICM delivery (open triangles) or IT delivery (solid triangles) at Day 1, Day 2, Day 4, Day 7, and Day 14, as indicated on the x-axis. ICM delivery was thereby shown to have resulted in an increase in % ID relative to IT delivery, at all time points.

FIG. 21B is a diagram showing the ROI assignments for the meninges versus whole brain, specifically indicating regions of partial overlap that include a portion of the parenchyma, the pia mater, and a portion of the subarachnoid space.

FIG. 22A is a graph showing the concentration of siRNA in μg equivalents/ml (y-axis) in CSF as assessed by gamma counting following either IT delivery (open squares) or ICM delivery (open triangles) at 24, 48, 96, 168, or 336 hours (x-axis).

FIG. 22B is a graph showing the concentration of siRNA in μg equivalents/ml (y-axis) in plasma as assessed by gamma counting following either IT delivery (open squares) or ICM delivery (open triangles) at 24, 48, 96, 168, or 336 hours (x-axis).

FIG. 22C is a modified version of the graph shown in FIG. 23B showing the concentration of siRNA in μg equivalents/ml (y-axis) in plasma as assessed by gamma counting following either IT delivery (open squares) or ICM delivery (open triangles) at 24, 48, 96, 168, or 336 hours (x-axis), where the x-axis is re-scaled to highlight the plasma siRNA profile in the first 48 hours.

### DETAILED DESCRIPTION

The present disclosure is based, at least in part, on the discovery of a method for delivering therapeutic oligonucleotides to the central nervous system (CNS) via intracisternal magna (ICM) administration, which results in specific and efficient knockdown of a target mRNA and associated protein product(s) within multiple CNS tissues and cells, including the lumbar spine, cervical spine, prefrontal cortex, hypothalamus, and striatum.

ICM involves administration (e.g., injection) into the cisterna magna, sometimes referred to as the cerebellomedullaris cistern. The brain has three principal openings—referred to as cisterns or cisterna—within the subarachnoid space: the cisterna interpeduncularis, the cisterna pontis, and the cisterna magna. The subarachnoid space is a cerebrospinal fluid (CSF) filled space, which is located between the arachnoid and the pia mater, covers the brain and continues down to the spinal cord. Like the subarachnoid space, the three cisterns are filled with CSF. The cisterna interpeduncularis and the cisterna pontis are located on the ventral side of the medulla oblongata, while the cisterna magna is located between the dorsal side of the medulla oblongata and the cerebellum.

As described herein, ICM administration may involve injection of a therapeutic oligonucleotide through the atlano-occipital membrane and into the cisterna magna space. The injection may include a stereotactic-guided injection system that includes a 3-way stopcock have a male Luer fitting and two female Luer fittings, or the injection may be infused by hand as a slow bolus. A 22-gauge 1 inch spinal needle may be attached to the male Luer lock fitting, a sterile syringe may be connected to the female Luer fitting opposite the male Luer lock fitting, and a tube may be connected to the second female Luer fitting. Optionally, the tube connected to the second female Luer fitting may be a loading line, which may in turn be connected to an infusion pump.

Generally, ICM delivery may involve aligning the spinal needle with the cervical gap of an anesthetized subject or patient, and then manually inserting the spinal needle through the skin and then through the atlanto-occipital membrane (positioned between the dorsal surface of the medulla oblongata and the cerebellum) and into the cisterna magna. To avoid damage to the medulla oblongata by over insertion of the spinal needle, it is important to verify the correct needle insertion depth. This may be accomplished by applying negative pressure to the needle (e.g., by partially retracting the syringe), and maintaining this negative pressure throughout the ICM administration. Once the spinal needle has passed through the atlanto-occipital membrane and into the cisterna magna, a small amount of CSF may be withdrawn into the syringe to serve as a baseline control if desired. The 3-way stopcock may then be opened to allow the loading line to deliver an oligonucleotide therapeutic at a desired rate (e.g., a slow bolus at 0.1 mL/minute-1 mL/minute) until the desired amount of therapeutic has been delivered (e.g., 25 mg, 50 mg, etc.), followed by an optional flush of the syringe with aCSF (e.g., 0.25 mL). After a resting period (e.g., about 5 minutes), the spinal needle may be slowly retracted out of the cisterna magna. Direct pressure may be used to achieve hemostasis after the spinal needle is completely removed. The subject or patient may then be monitored until full recovery from the anesthesia.

Advantageously, the ICM administration techniques disclosed herein allow for targeted mRNA knockdown in the striatum, which is not in direct contact with the CSF, but is instead surrounded by brain interstitial fluid (ISF). The striatum is a part of the thalamocortical system, residing in a loop that connects the cortex to the thalamus. Previous methods of drug delivery to striatum relied mainly on intraparenchymal administration (i.e., direct injection into the striatum), which has the disadvantage of being an extremely invasive procedure because the striatum is located deep within the brain. In this regard, ICM administration of therapeutic oligonucleotides to the CNS via ICM administration has the advantage of being able to deliver therapeutic oligonucleotides to regions of the CNS that are known to be refractory to direct delivery protocols.

As described in detail below, the present disclosure provides conjugated lipophilic moieties on internal position(s) of a double-stranded iRNA agent(s) having a non-limiting pattern of modified nucleotides that sufficiently directed the iRNA agent(s) to CNS tissues such that ICM injection of the iRNA agent(s) enabled targeting to, and uptake by, tissues and cells of the lumbar spine, cervical spine, prefrontal cortex, hypothalamus, and striatum, which then induced significant and sustained mRNA knockdown of the target mRNA of interest. The ability of ICM administration of therapeutic oligonucleotides to effectively knockdown target mRNA levels in such a variety of CNS tissues was both surprising and unexpected. Importantly, the techniques herein provide the ability to significantly reduce expression of a target gene in multiple CNS tissues following a single ICM administration. For example, the expression of a target gene may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Additionally, as described in detail below, the reduction of target gene expression occurs over a clinically relevant time period. For example, the expression of the target gene may be reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the expression of the target gene is reduced by about 20%, about 25%, about 30%, about 35%, about 40%, about 45%, about 50%, about 55%, about 60%, about 65%, about 70%, or about 75% within about 29 days. In some embodiments, the expression of the target gene is reduced by about 25% to about 35% within about 29 days. In some embodiments, the expression of the target gene is reduced by about 35% to about 45% within about 29 days. In some embodiments, the expression of the target gene is reduced by about 45% to about 55% within about 29 days. In some embodiments, the expression of the target gene is reduced by about 25% to about 50% within about 29 days. In some embodiments, the expression of the target gene is reduced by about 35% to about 50% within about 29 days. In some embodiments, the expression of the target gene is reduced by about 45% to about 50% within about 29 days. In some embodiments, the expression of the target gene is reduced by about 50% within about 29 days. In some embodiments, the expression of the target gene is reduced by about 25% to about 50% within about 25 to about 31 days. In some embodiments, the expression of the target gene is reduced by about 35% to about 50% within about 25 to about 31 days. In some embodiments, the expression of the target gene is reduced by about 45% to about 50% within about 25 to about 31 days. In some embodiments, the expression of the target gene is reduced by about 50% within about 25 to about 31 days.

The disclosure has identified, inter alia, that conjugating a lipophilic moiety to one or more internal positions on at least one strand of the double-stranded iRNA agent provides surprisingly good results for in vivo intravitreal delivery and intrathecal delivery and intracisternal magna delivery of the double-stranded iRNAs, resulting in efficient entry of CNS tissues and ocular tissues and are efficiently internalized into cells of the CNS system and ocular system. Advantageously, the techniques herein provide the ability to deliver therapeutic agents (e.g., double-stranded iRNA agents) into the lumbar spine, cervical spine, prefrontal cortex, hypothalamus, and striatum. For example, in some embodiments, the techniques herein provide the ability to deliver double-stranded iRNA agents directed to a target gene selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25 into the cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG). In some embodiments, the target gene is not HTT.

Importantly, the delivery techniques herein provide the ability to detect a double-stranded iRNA agent in the lumbar spine, cervical spine, prefrontal cortex, hypothalamus, and striatum tissues or cells after ICM delivery has occurred. For example, in some embodiments the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the double-stranded iRNA agent is detected within about 1 to about 5 days, about 5 to about 10 days, about 10 to about 15 days, about 15 to about 20 days, about 20 to about 25 days, or about 25 to about 30 days. In some embodiments, the double-stranded iRNA agent is detected within about 25 days to about 30 days. In some embodiments, the double-stranded iRNA agent is detected within about 29 days. In some embodiments, the double-stranded iRNA agent is detected after at least 29 days. In some embodiments, the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, the double-stranded iRNA agent is detected after about 1 to about 5 days, about 5 to about 10 days, about 10 to about 15 days, about 15 to about 20 days, about 20 to about 25 days, or about 25 to about 30 days. In some embodiments, the double-stranded iRNA agent is detected after about 25 days to about 30 days. In some embodiments, the double-stranded iRNA agent is detected after about 29 days. In some embodiments, the expression of the target gene is reduced by at least 96%, at least 97%, at least 98%, at least 99%, or at least 100%. In some embodiments, the expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

One aspect of the invention provides a double-stranded iRNA agent that includes: an antisense strand which is complementary to a target gene; a sense strand which is complementary to said antisense strand; and one or more lipophilic moieties conjugated to one or more internal positions on at least one strand, optionally via a linker or carrier.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the double-stranded iRNA agent, conjugated to one or more lipophilic moieties, can be measured by its protein binding characteristics. For instance, the unbound fraction in the plasma protein binding assay of the double-stranded iRNA agent can be determined to positively correlate to the relative hydrophobicity of the double-stranded iRNA agent, which can positively correlate to the silencing activity of the double-stranded iRNA agent.

In one embodiment, the plasma protein binding assay determined is an electrophoretic mobility shift assay (EMSA) using human serum albumin protein. An exemplary protocol of this binding assay is illustrated in detail in Example 14. The hydrophobicity of the double-stranded iRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded iRNA agent provides optimal hydrophobicity for the enhanced in vivo delivery of siRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprises a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic moiety may be attached to the iRNA agent by any method known in the art, including via a functional grouping already present in the lipophilic moiety or introduced into the iRNA agent, such as a hydroxy group (e.g., —CO—CH₂—OH). The functional groups already present in the lipophilic moiety or introduced into the iRNA agent include, but are not limited to, hydroxyl, amine, carboxylic acid, sulfonate, phosphate, thiol, azide, and alkyne.

Conjugation of the iRNA agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the double-stranded iRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglobulin, or α-1-glycoprotein.

In certain embodiments, the ligand is naproxen or a structural derivative of naproxen. In certain embodiments, the ligand is ibuprofen or a structural derivative of ibuprofen.

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In certain embodiments, more than one lipophilic moieties can be incorporated into the double-strand iRNA agent, particularly when the lipophilic moiety has a low lipophilicity or hydrophobicity. In one embodiment, two or more lipophilic moieties are incorporated into the same strand of the double-strand iRNA agent. In one embodiment, each strand of the double-strand iRNA agent has one or more lipophilic moieties incorporated. In one embodiment, two or more lipophilic moieties are incorporated into the same position (i.e., the same nucleobase, same sugar moiety, or same internucleosidic linkage) of the double-strand iRNA agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the iRNA agent via a direct attachment to the ribosugar of the iRNA agent. Alternatively, the lipophilic moiety may be conjugated to the double-strand iRNA agent via a linker or a carrier.

In one embodiment, the ligand is conjugated at the 2′-position of a nucleotide or modified nucleotide within the sense or antisense strand. For example, a C16 ligand may be conjugated as shown in the following structure:

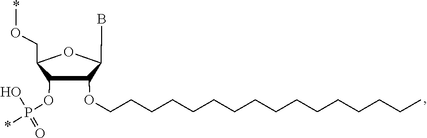

where * denotes a bond to an adjacent nucleotide, and B is a nucleobase or a nucleobase analog, optionally where B is adenine, guanine, cytosine, thymine or uracil.

In certain embodiments, the lipophilic moiety may be conjugated to the iRNA agent via one or more linkers (tethers).

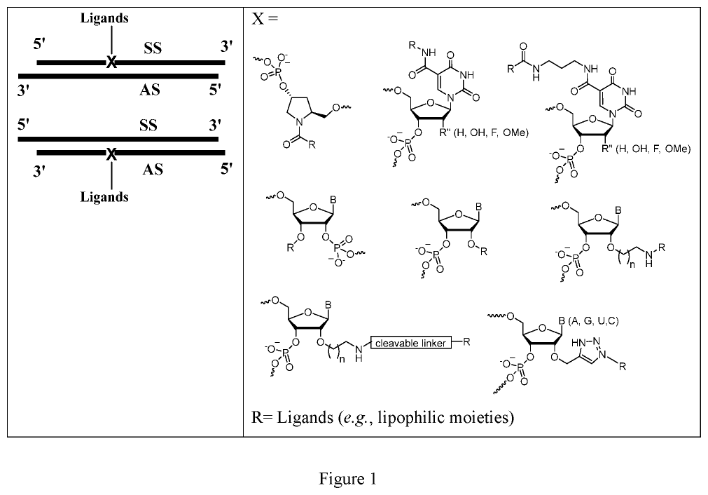

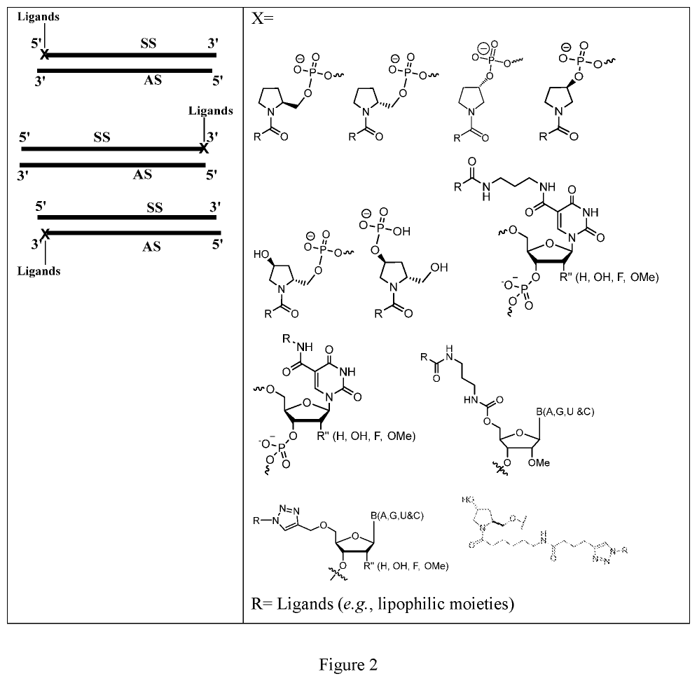

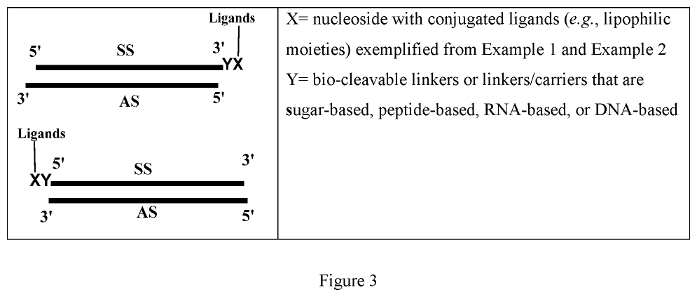

In one embodiment, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate. Some exemplary linkages are illustrated in FIG. 1, Examples 2, 3, 5, 6, and 7.

### Linkers/Tethers

Linkers/Tethers are connected to the lipophilic moiety at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (italics) include TAP-(CH₂)ₙNH—; TAP—C(O)(CH₂)ₙNH—; TAP—NR″″(CH₂)ₙNH—, TAP—C(O)—(CH₂)ₙ—C(O)—; TAP—C(O)—(CH₂)ₙ—C(O)O—; TAP—C(O)—O—; TAP—C(O)—(CH₂)ₙ—NH—C(O)—; TAP—C(O)—(CH₂)ₙ—; TAP—C(O)—NH—; TAP—C(O)—; TAP—(CH₂)ₙ—C(O)—; TAP—(CH₂)ₙ—C(O)O—; TAP—(CH₂)ₙ—; or TAP—(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP—(CH₂)ₙNH(LIGAND); TAP—C(O)(CH₂)ₙNH(LIGAND); TAP-NR″″(CH₂)ₙNH(LIGAND); TAP—(CH₂)ₙONH(LIGAND); TAP—C(O)(CH₂)ₙONH(LIGAND); TAP—NR″″(CH₂)ₙONH(LIGAND); TAP—(CH₂)ₙNHNH₂(LIGAND), TAP—C(O)(CH₂)ₙNHNH₂(LIGAND); TAP—NR″″(CH₂)ₙNHNH₂(LIGAND); TAP—C(O)—(CH₂)ₙ—C(O)(LIGAND); TAP—C(O)—(CH₂)ₙ—C(O)O(LIGAND); TAP—C(O)—O(LIGAND); TAP—C(O)—(CH₂)ₙ—NH—C(O)(LIGAND); TAP—C(O)—(CH₂)ₙ(LIGAND); TAP—C(O)—NH(LIGAND); TAP—C(O)(LIGAND); TAP—(CH₂)ₙ—C(O) (LIGAND); TAP—(CH₂)ₙ—C(O)O(LIGAND); TAP—(CH₂)ₙ(LIGAND); or TAP—(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can be acylated, e.g., with C(O)CF₃.

In some embodiments, the linker/tether can terminate with a mercapto group (i.e., SH) or an olefin (e.g., CH═CH₂). For example, the tether can be TAP—(CH₂)ₙ—SH, TAP—C(O)(CH₂)ₙSH, TAP—(CH₂)ₙ—(CH═CH₂), or TAP—C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP—(CH₂)ₙCHO; TAP—C(O)(CH₂)ₙCHO; or TAP—NR″″(CH₂)ₙCHO, in which n is 1-6 and R″″ is C₁-C₆ alkyl; or TAP—(CH₂)ₙC(O)ONHS; TAP—C(O)(CH₂)ₙC(O)ONHS; or TAP-NR″″(CH₂)ₙC(O)ONHS, in which n is 1-6 and R″″ is C₁-C₆ alkyl; TAP—(CH₂)ₙC(O)OC₆F₅; TAP—C(O)(CH₂)ₙC(O) OC₆F₅; or TAP—NR″″(CH₂)ₙC(O) OC₆F₅, in which n is 1-11 and R″″ is C₁-C₆ alkyl; or —(CH₂)ₙCH₂LG; TAP—C(O)(CH₂)ₙCH₂LG; or TAP—NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

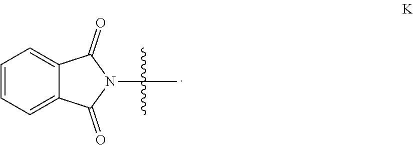 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets cells in the CNS can be conjugated to a tether that includes a sialic acid (SA). CNS cells are enriched for neuramidase enzymes (e.g., neuramidase 1 (NEU1), neuramidase 2 (NEU2), neuramidase 3 (NEU3), neuramidase 4 (NEU4), and the like). In particular, NEU3 is enriched in the cells of the CNS and is localized to the inner membrane of the nuclear envelope while NEU1 is localized to the outer membrane of the nuclear envelope, as well as the plasma membrane (see e.g., Ledeen et al. (2011) New findings on nuclear gangliosides: overview on metabolism and function. 116(5):714-720). NEU3 cleaves terminal 2,3- and 2,6-linked SA (see e.g., U.S. Pat. No. 10,907,176). For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers Tethers

The linkers can also include biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

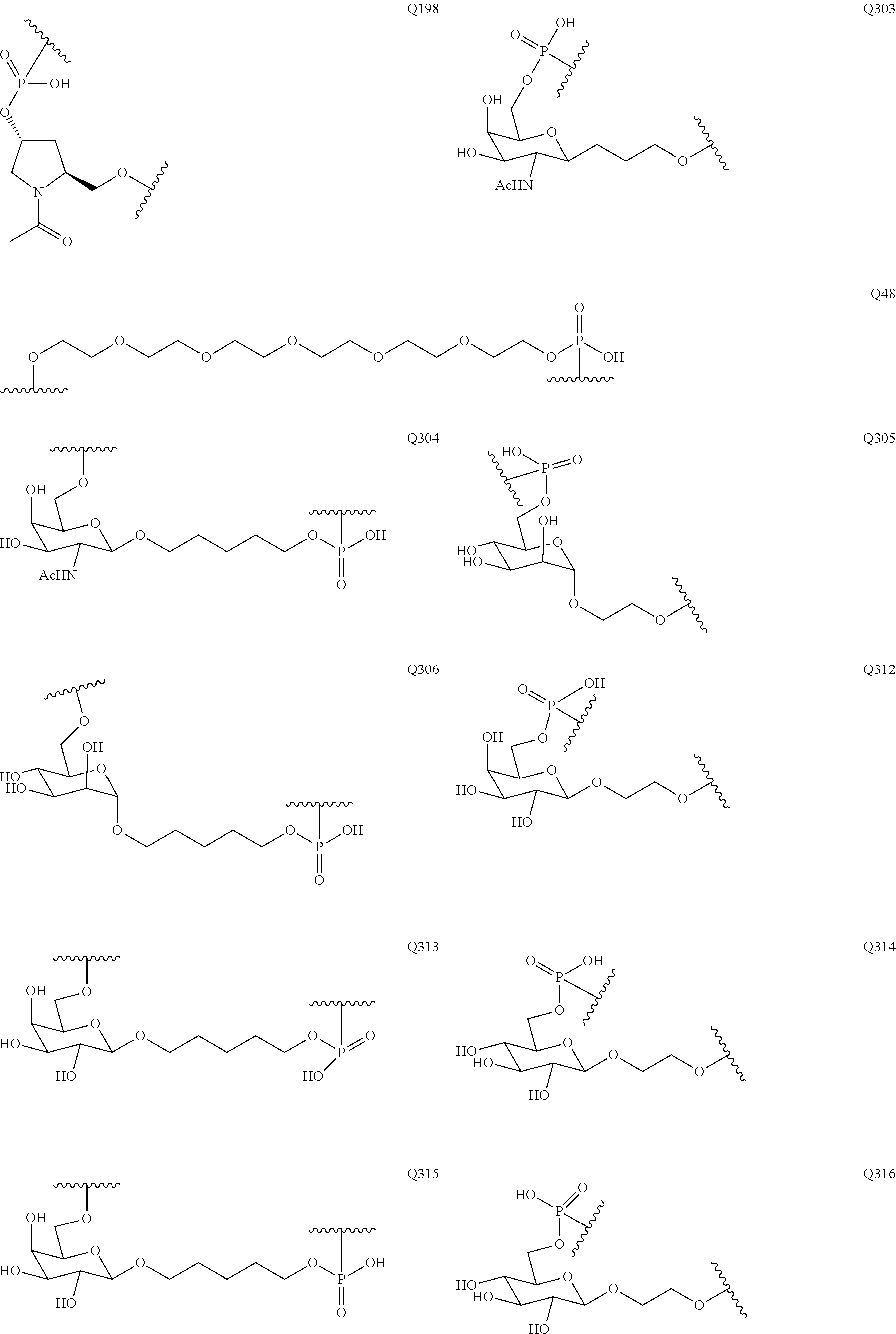

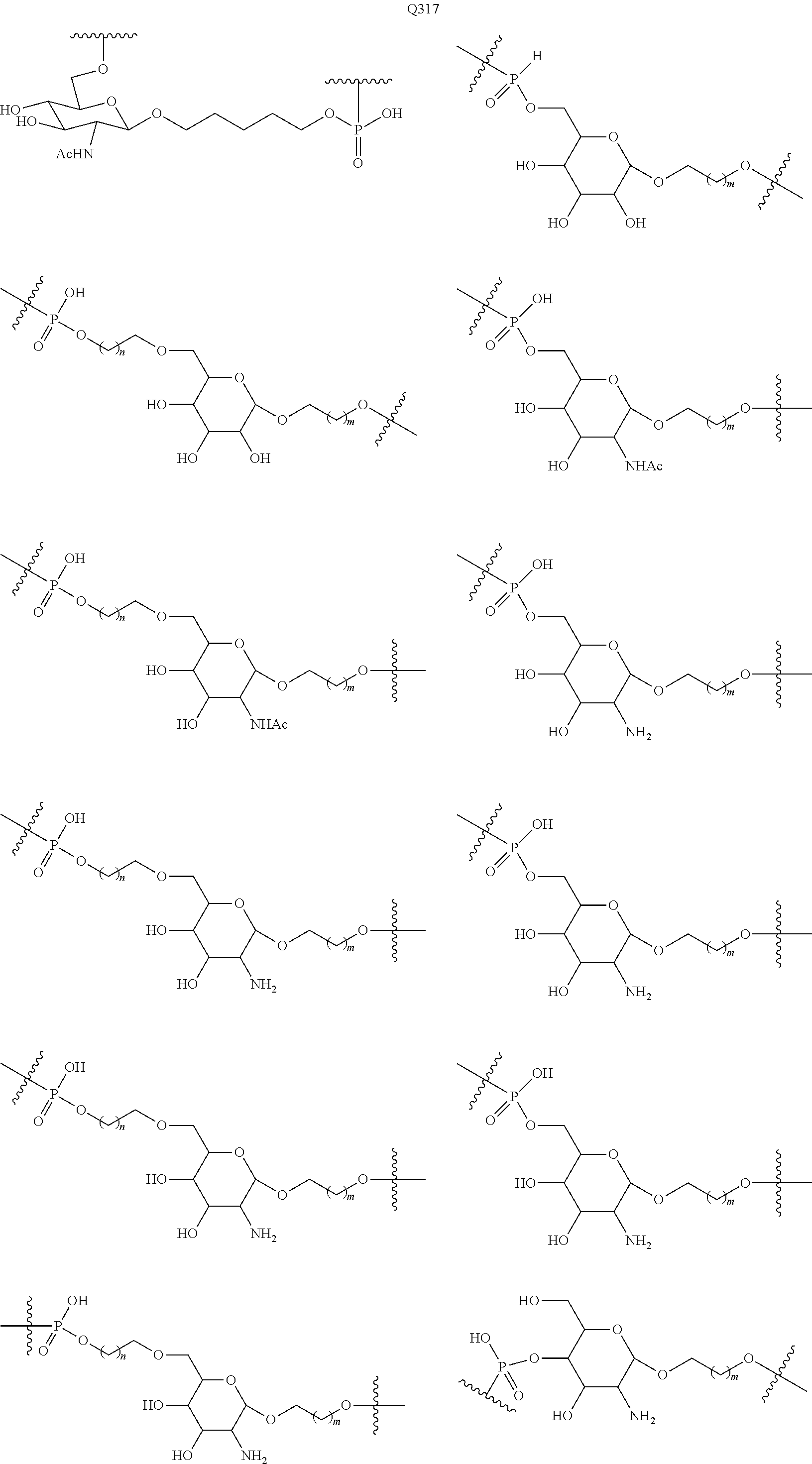

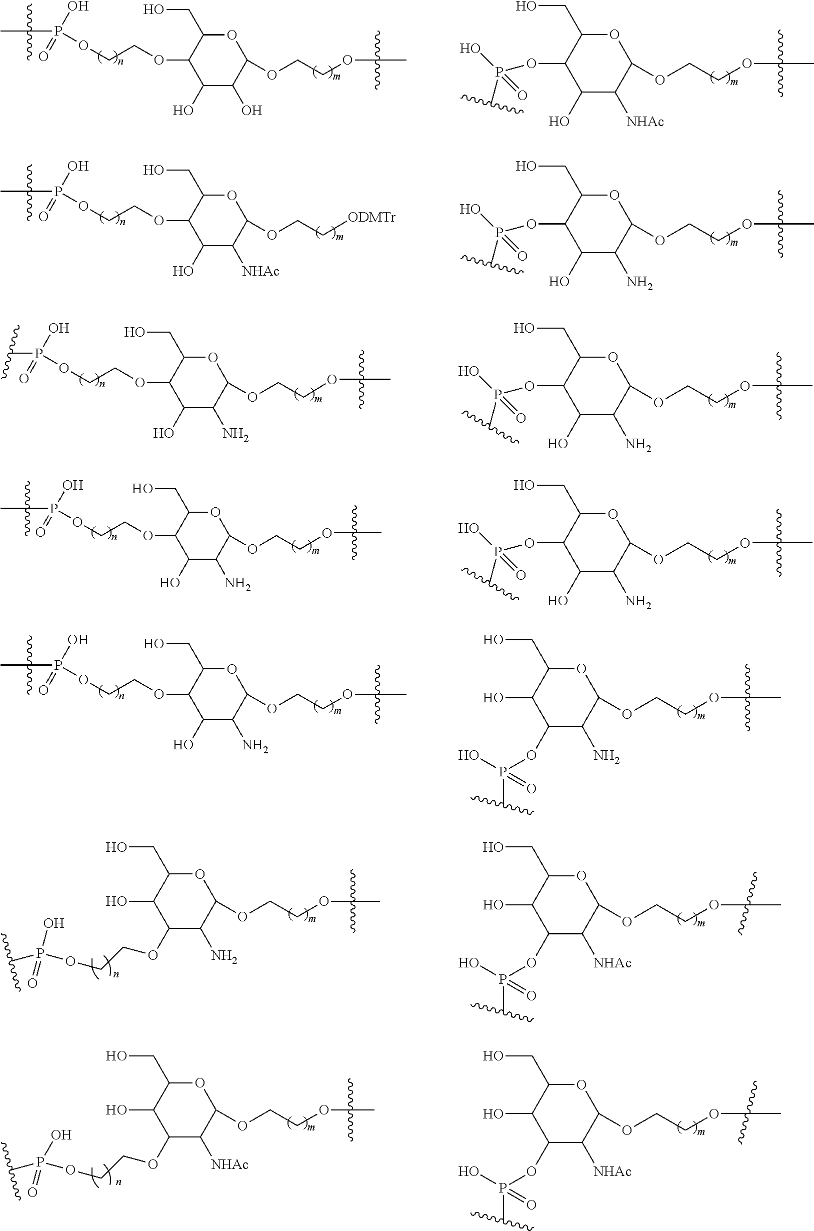

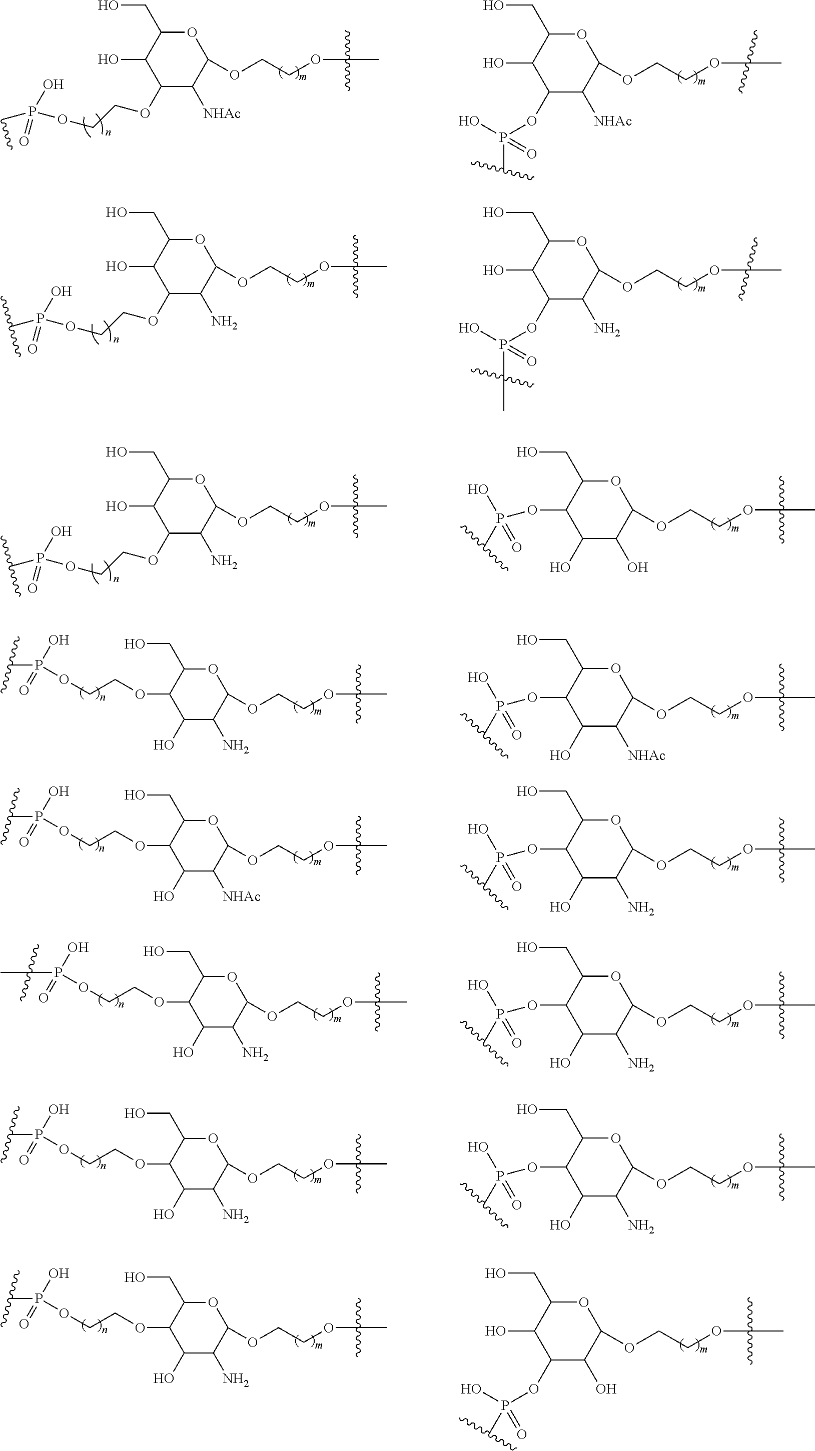

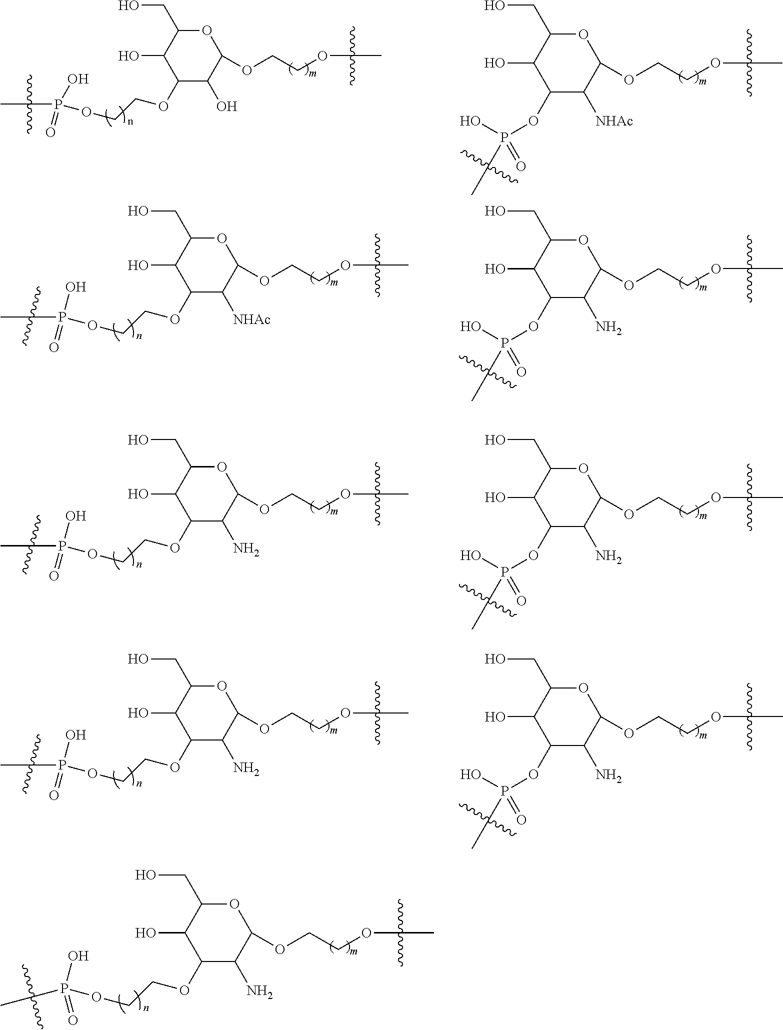

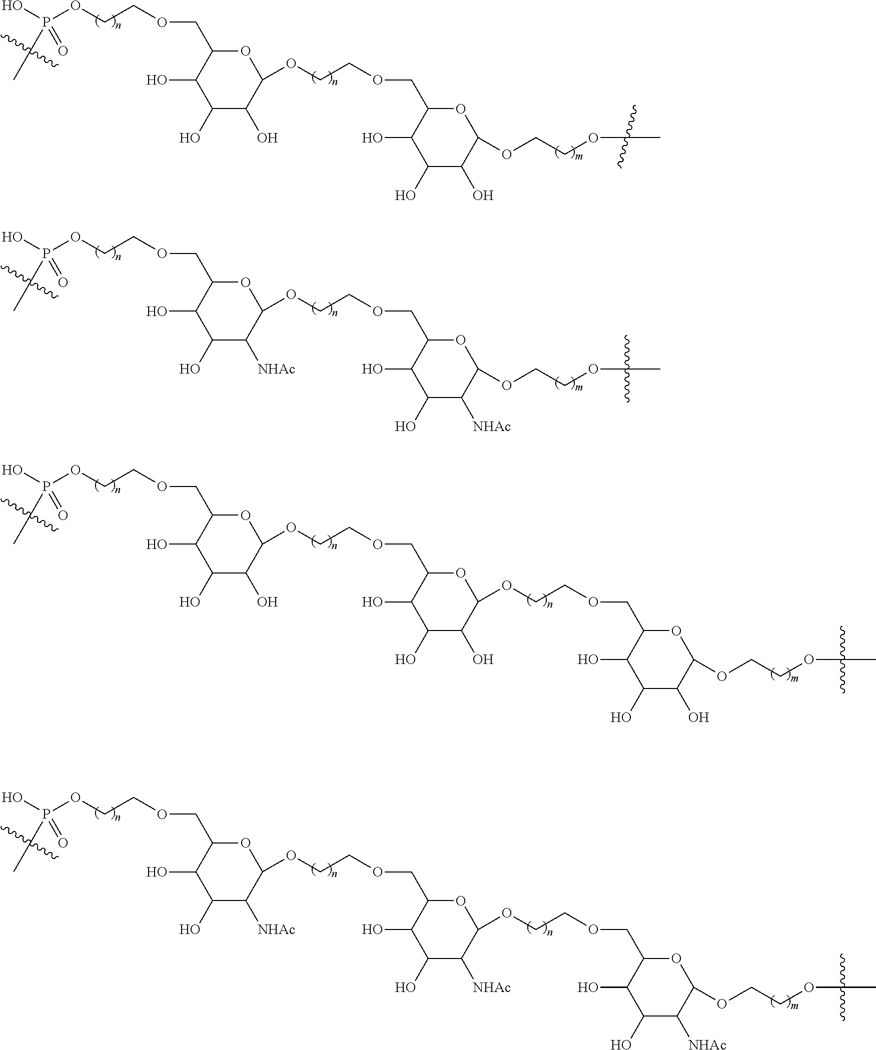

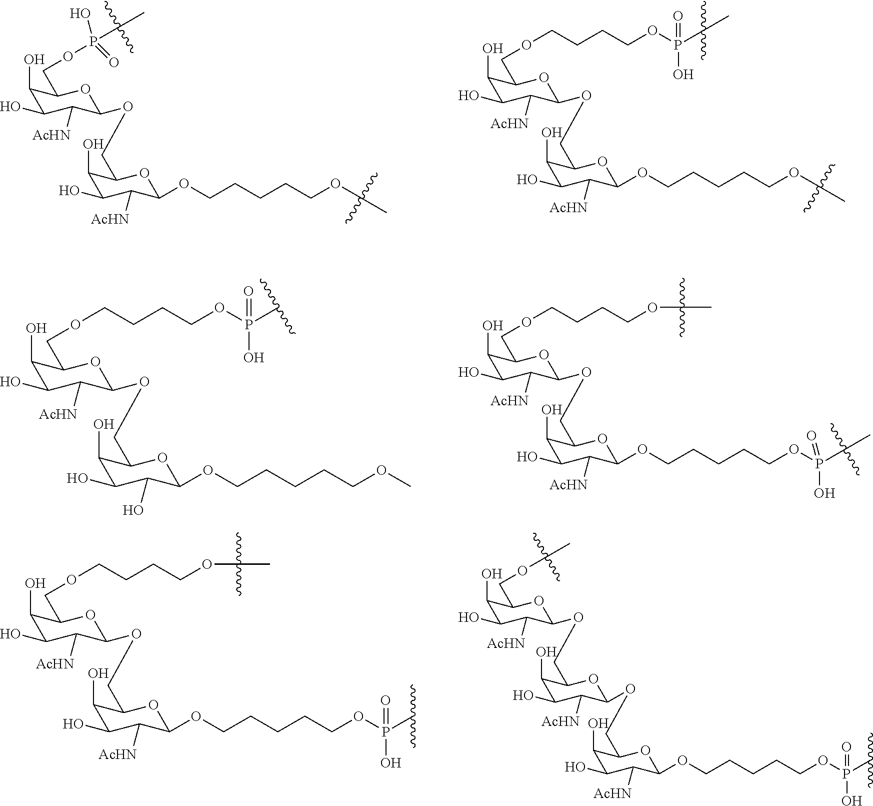 [SMILES: *OCCCCCOC1OC(COC2OC(COP(*)(=O)O)C(O)C(O)C2NC(C)=O)C(O)C(O)C1NC(C)=O ; *OCCCCCOC1OC(COC2OC(COCCCCOP(*)(=O)O)C(O)C(O)C2NC(C)=O)C(O)C(O)C1NC(C)=O ; *P(=O)(O)OCCCCOCC1OC(OCC2OC(OCCCCCOC)C(O)C(O)C2O)C(NC(C)=O)C(O)C1O ; *OCCCCOCC1OC(OCC2OC(OCCCCCOP(*)(=O)O)C(NC(C)=O)C(O)C2O)C(NC(C)=O)C(O)C1O ; *OCCCCOCC1OC(OCC2OC(OCCCCCOP(*)(=O)O)C(NC(C)=O)C(O)C2O)C(NC(C)=O)C(O)C1O ; *OCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O]

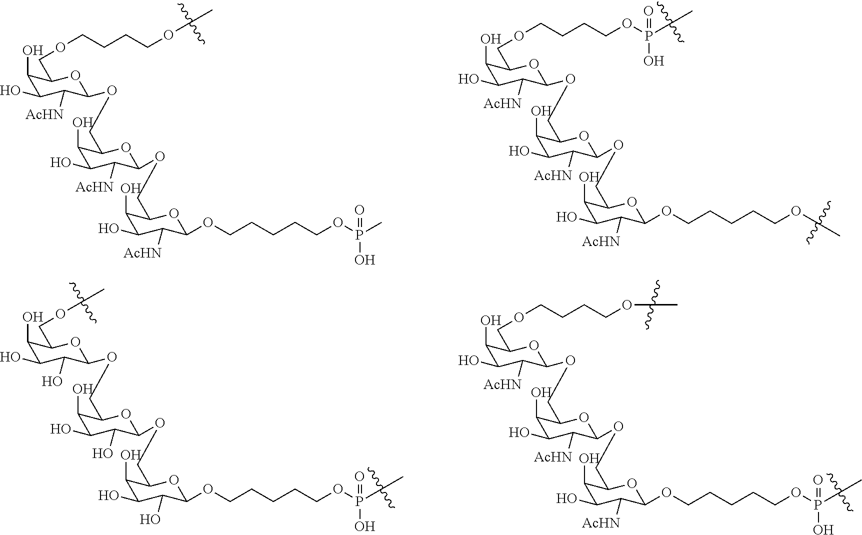 [SMILES: *OCCCCOCC1OC(OCC2OC(OCC3OC(OCCCCCOP(C)(=O)O)C(NC(C)=O)C(O)C3O)C(NC(C)=O)C(O)C2O)C(NC(C)=O)C(O)C1O ; *OCCCCCOC1OC(COC2OC(COC3OC(COCCCCOP(*)(=O)O)C(O)C(O)C3NC(C)=O)C(O)C(O)C2NC(C)=O)C(O)C(O)C1NC(C)=O ; *OCC1OC(OCC2OC(OCC3OC(OCCCCCOP(*)(=O)O)C(O)C(O)C3O)C(O)C(O)C2O)C(O)C(O)C1O ; *OCCCCOCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O]

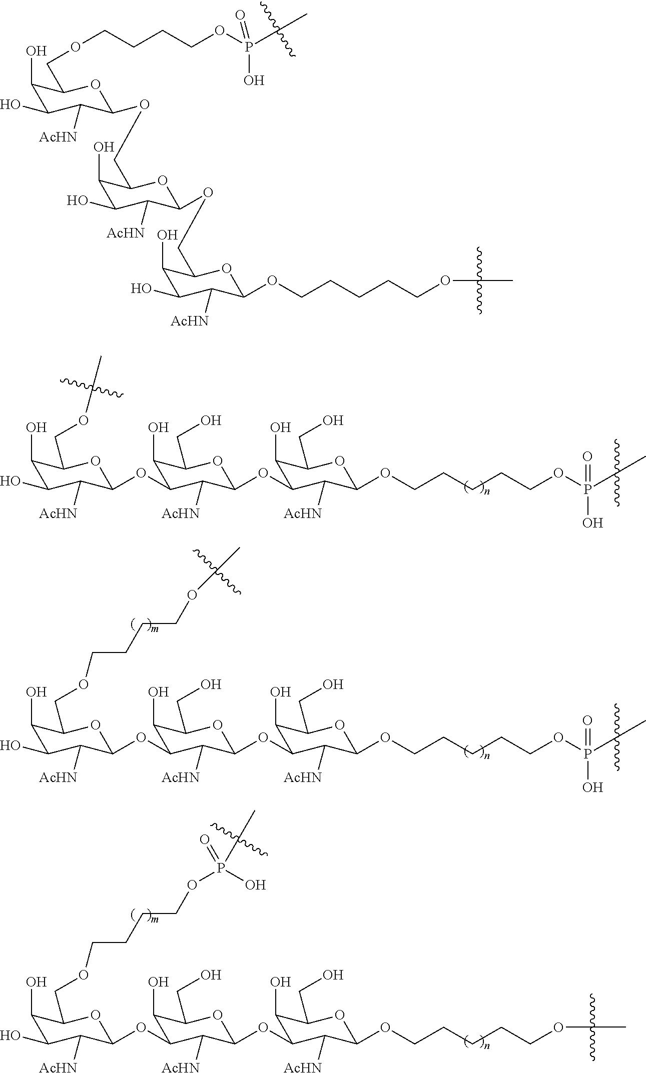

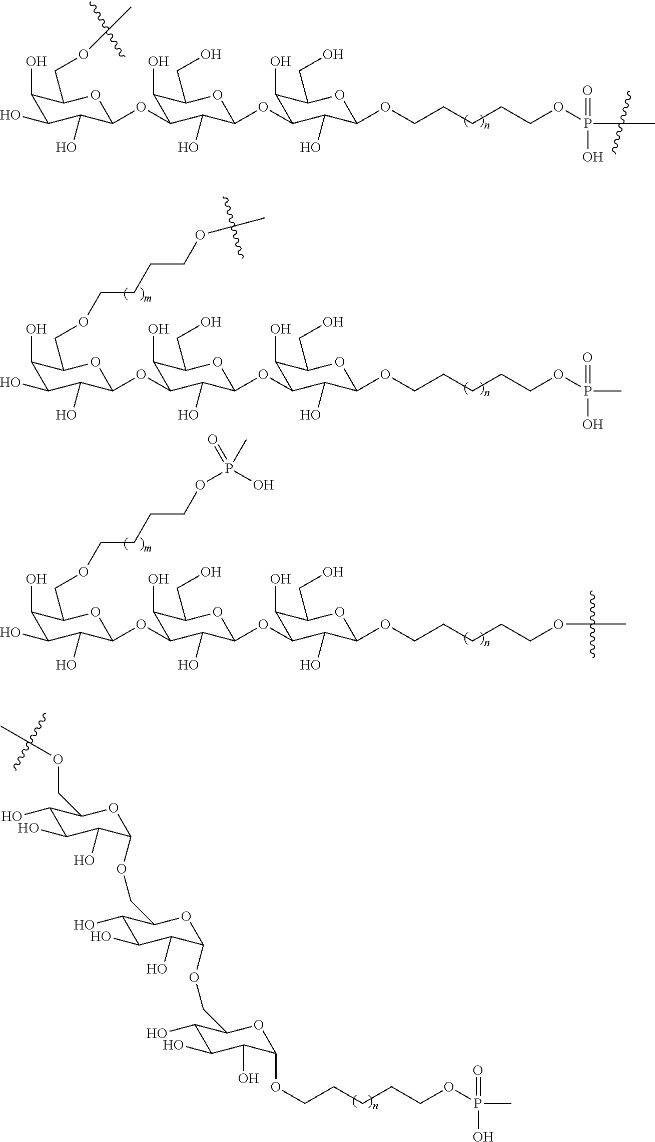

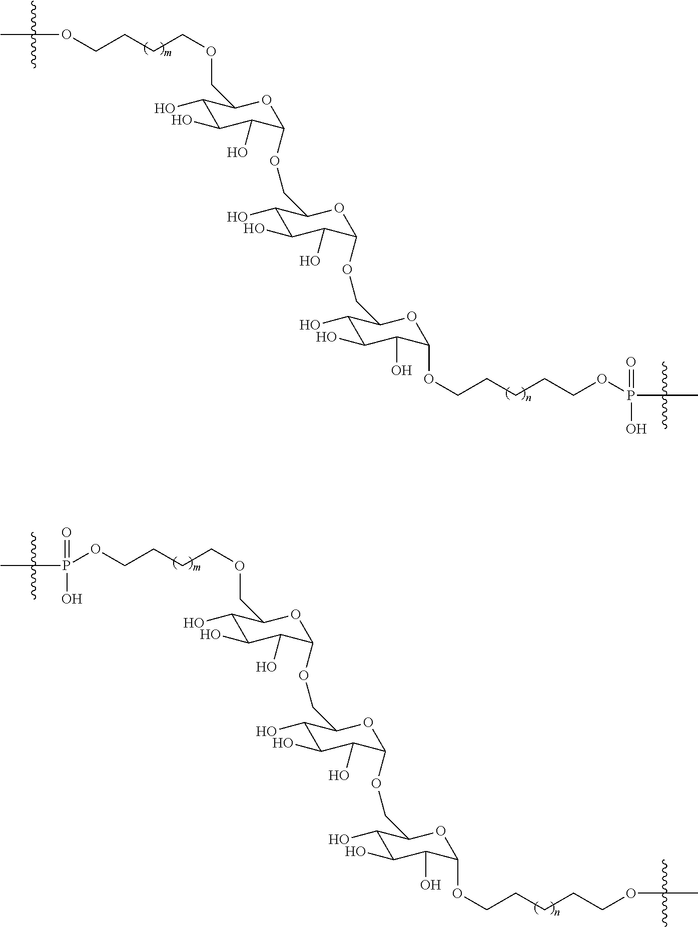

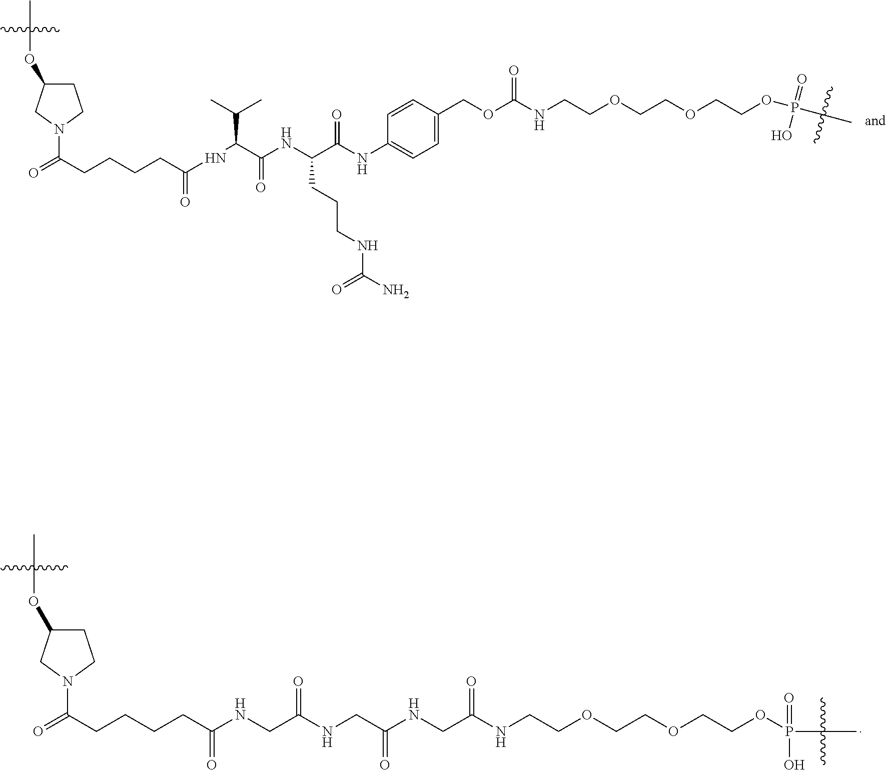 [SMILES: *O[C@H]1CCN(C(=O)CCCCC(=O)N[C@H](C(=O)N[C@@H](CCCNC(N)=O)C(=O)Nc2ccc(COC(=O)NCCOCCOCCOP(*)(=O)O)cc2)C(C)C)C1 ; *O[C@H]1CCN(C(=O)CCCCC(=O)NCC(=O)NCC(=O)NCC(=O)NCCOCCOCCOP(*)(=O)O)C1]

Additional exemplary bio-cleavable linkers are illustrated in Schemes 28-30.

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carriers

In certain embodiments, the lipophilic moiety is conjugated to the iRNA agent via a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The lipophilic moiety can be directly attached to the carrier or indirectly attached to the carrier by an intervening linker/tether, as described above.

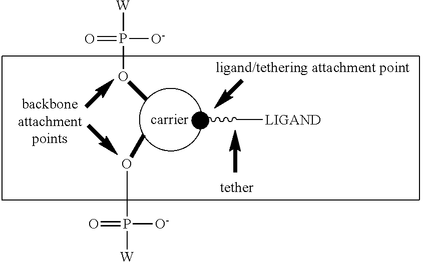

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

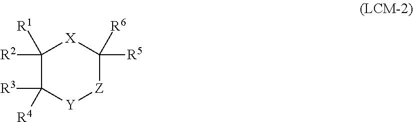

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

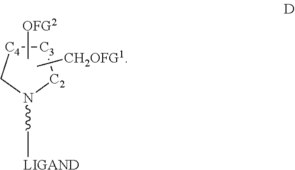

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

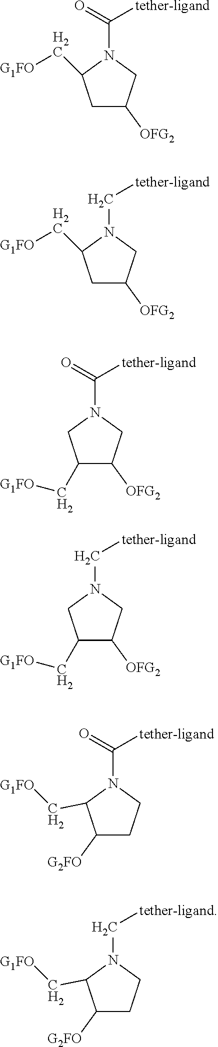

In certain embodiments, the carrier may be based on the piperidine ring system (E), e.g., X is N(CO)R⁷ or NR⁷, Y is CR⁹R¹⁰, and Z is CR¹¹R¹².

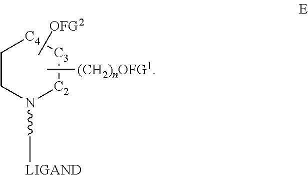

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

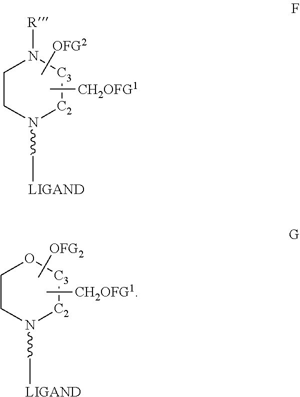

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

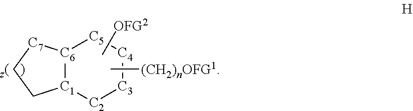

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

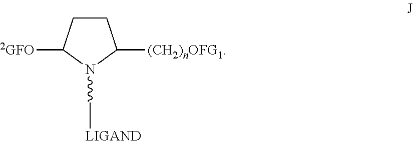

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

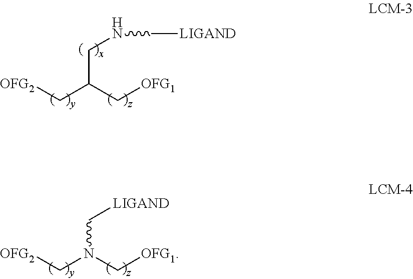

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the double stranded iRNA agent comprises one or more lipophilic moieties conjugated to the 5′ end of the sense strand or the 5′ end of the antisense strand.

In certain embodiments, the lipophilic moiety is conjugated to the 5′-end of a strand via a carrier and/or linker. In one embodiment, the lipophilic moiety is conjugated to the 5′-end of a strand via a carrier of a formula:

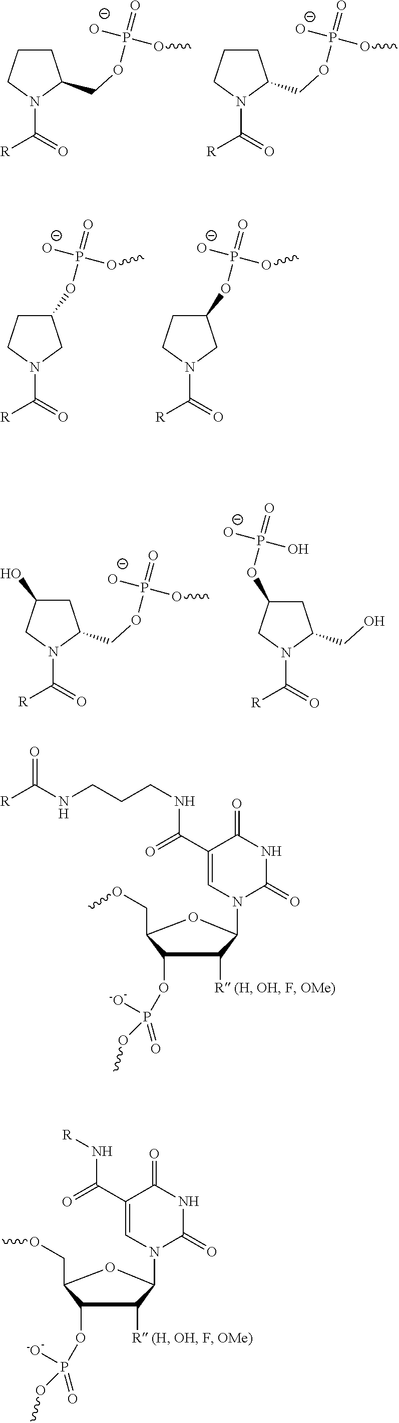

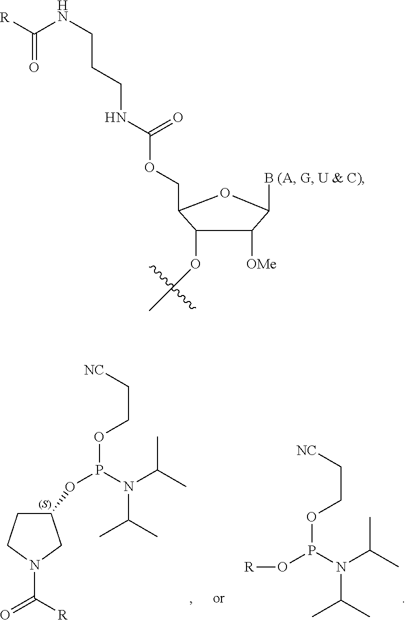

R is a ligand such as the lipophilic moiety.

In some embodiments, the double stranded iRNA agent comprises one or more lipophilic moieties conjugated to the 3′ end of the sense strand or the 3′ end of the antisense strand.

In certain embodiments, the lipophilic moiety is conjugated to the 3′-end of a strand via a carrier and/or linker. In one embodiment, the lipophilic moiety is conjugated to the 3′-end of a strand via a carrier of a formula:

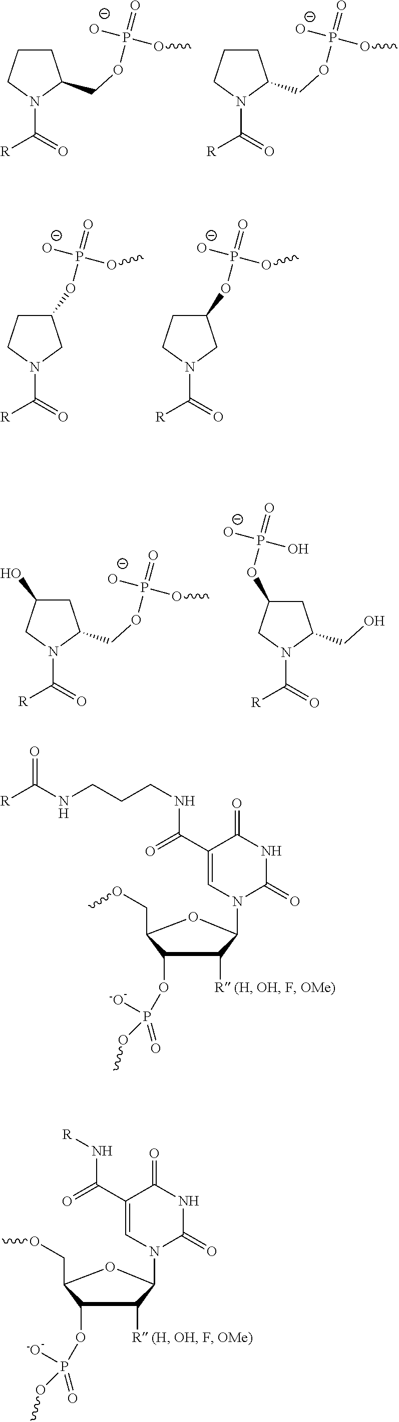

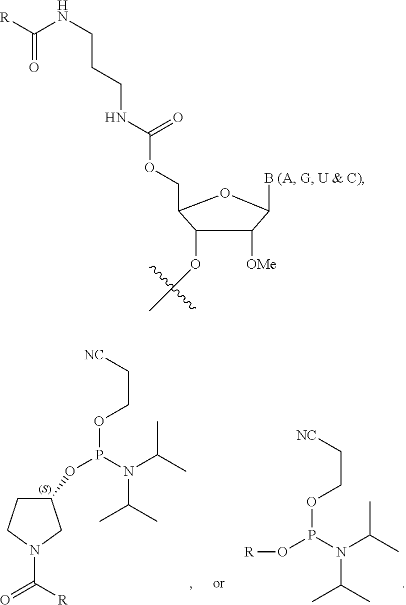

R is a ligand such as the lipophilic moiety.

In some embodiments, the double stranded iRNA agent comprises one or more lipophilic moieties conjugated to both ends of the sense strand.

In some embodiments, the double stranded iRNA agent comprises one or more lipophilic moieties conjugated to both ends of the antisense strand.

In some embodiments, the double stranded iRNA agent comprises one or more lipophilic moieties conjugated to the 5′ end or 3′ end of the sense strand, and one or more lipophilic moieties conjugated to the 5′ end or 3′ end of the antisense strand,

In some embodiments, the lipophilic moiety is conjugated to the terminal end of a strand via one or more linkers (tethers) and/or a carrier.

In one embodiment, the lipophilic moiety is conjugated to the terminal end of a strand via one or more linkers (tethers).

In one embodiment, the lipophilic moiety is conjugated to the 5′ end of the sense strand or antisense strand via a cyclic carrier, optionally via one or more intervening linkers (tethers).

In some embodiments, the lipophilic moiety is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the lipophilic moiety is conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the lipophilic moiety is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the lipophilic moiety is conjugated to one or more internal positions on at least one strand, except the cleavage site region of the sense strand, for instance, the lipophilic moiety is not conjugated to positions 9-12 counting from the 5′-end of the sense strand. Alternatively, the internal positions exclude positions 11-13 counting from the 3′-end of the sense strand.

In one embodiment, the lipophilic moiety is conjugated to one or more internal positions on at least one strand, which exclude the cleavage site region of the antisense strand. For instance, the internal positions exclude positions 12-14 counting from the 5′-end of the antisense strand.

In one embodiment, the lipophilic moiety is conjugated to one or more internal positions on at least one strand, which exclude positions 11-13 on the sense strand, counting from the 3′-end, and positions 12-14 on the antisense strand, counting from the 5′-end.

In one embodiment, one or more lipophilic moieties are conjugated to one or more of the following internal positions: positions 4-8 and 13-18 on the sense strand, and positions 6-10 and 15-18 on the antisense strand, counting from the 5′end of each strand.

In one embodiment, one or more lipophilic moieties are conjugated to one or more of the following internal positions: positions 5, 6, 7, 15, and 17 on the sense strand, and positions 15 and 17 on the antisense strand, counting from the 5′end of each strand.

In one embodiment, one or more lipophilic moieties are conjugated to one or more of the following internal positions: positions 4-8 and 13-18 on the sense strand, counting from the 5′end of the strand.

In one embodiment, one or more lipophilic moieties are conjugated to one or more of the following internal positions: positions 5, 6, 7, 15, and 17 (e.g., position 6) on the sense strand, counting from the 5′end of the strand.

In one embodiment, one or more lipophilic moieties are conjugated to one or more of the following internal positions: positions 6-10 and 15-18 on the antisense strand, counting from the 5′end of the strand.

In one embodiment, one or more lipophilic moieties are conjugated to one or more of the following internal positions: positions 15 and 17 on the antisense strand, counting from the 5′end of each strand.

In some embodiments, the lipophilic moiety is conjugated to a nucleobase, sugar moiety, or internucleosidic linkage of the double-stranded iRNA agent.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the overall double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{[ \begin{matrix}expression with siRNA - \\ expression without siRNA\end{matrix} ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded iRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, /. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each 15 to 30 nucleotides in length.

In one embodiment, the sense and antisense strands of the double-stranded iRNA agent are each 19 to 25 nucleotides in length.

In one embodiment, the sense and antisense strands of the double-stranded iRNA agent are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the double-stranded iRNA agent comprises a single-stranded overhang on at least one of the termini. In one embodiment, the single-stranded overhang is 1, 2, or 3 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the double-stranded iRNA has a ZXY structure, such as is described in PCT Publication No. 2004080406, which is hereby incorporated by reference in its entirety.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

In certain embodiments, two oligomeric strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

As used herein, “subarachnoid space” refers to a space that exists between the arachnoid and the pia mater, and continues down the spinal cord. The subarachnoid space includes three openings, referred to as cisterns, which include: the cisterna interpeduncularis, the cisterna pontis, and the cisterna magna. The subarachnoid space, as well as each of the three cisterns, are filled with cerebrospinal fluid (CSF).

The term “or” is used herein to mean, and is used interchangeably with, the term “and/or,” unless context clearly indicates otherwise.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

siRNA Design

In one embodiment, the iRNA agent of the invention is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the iRNA agent of the invention is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the iRNA agent of the invention is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the iRNA agent of the invention comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the iRNA is blunt, while the other end is comprises a 2 nt overhang. Preferably, the 2 nt overhang is at the 3′-end of the antisense.

In one embodiment, the iRNA agent of the invention comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent of the invention comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal.

In one embodiment, the sense strand of the iRNA agent contains at least one motif of three identical modifications on three consecutive nucleotides, where one of the motifs occurs at the cleavage site in the sense strand.

In one embodiment, the antisense strand of the iRNA agent can also contain at least one motif of three identical modifications on three consecutive nucleotides, where one of the motifs occurs at or near the cleavage site in the antisense strand

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In one embodiment, the iRNA agent of the invention comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In one aspect, the invention relates to a double-stranded RNA (dsRNA) agent for inhibiting the expression of a target gene. The dsRNA agent comprises a sense strand and an antisense strand, each strand having 14 to 40 nucleotides. The dsRNA agent is represented by formula (I):

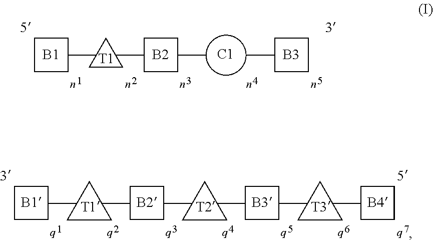

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

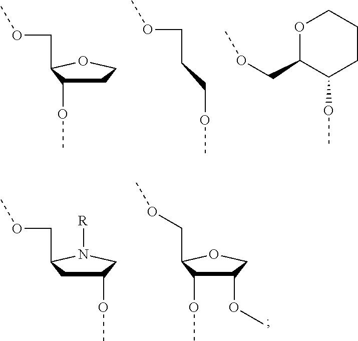

and iii) sugar modification selected from the group consisting of:

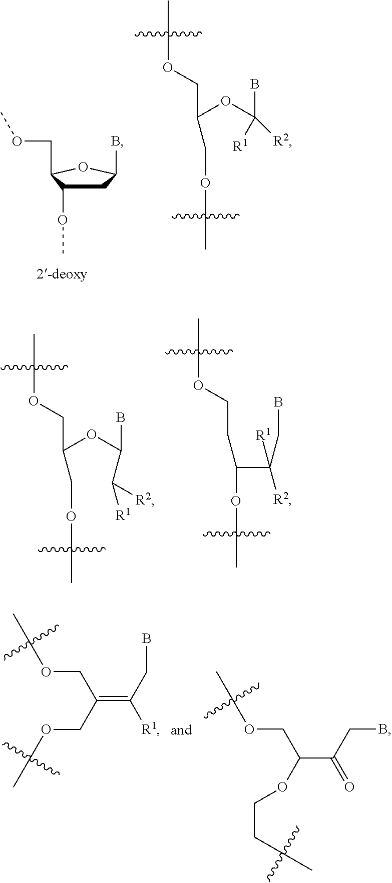

wherein B is a modified or unmodified nucleobase, R¹ and R² independently are H, halogen, OR₃, or alkyl; and R³ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

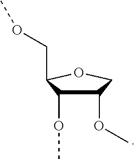

In some embodiments the thermally destabilizing modification of the duplex is selected from the group consisting of:

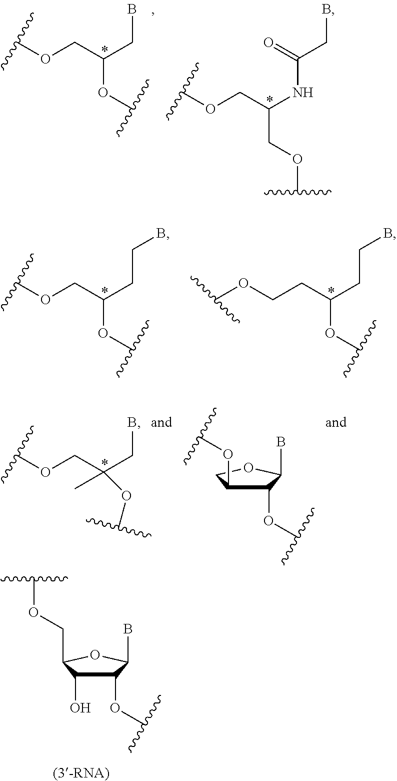

wherein B is a modified or unmodified nucleobase and the asterisk on each structure represents either R, S or racemic.

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0.

q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end Vinyl phosphonate (5′-VP), 5′-end methylphosphonate (MePhos), or 5′-deoxy-5′-C-malonyl (

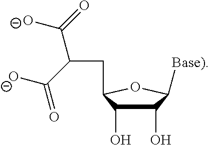

When the 5′-end phosphorus-containing group is 5′-end Vinyl phosphonate (5′-VP), the 5′-VP can be either 5′-E-VP isomer (i.e., trans-vinylphosphate,

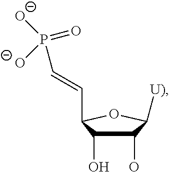

5′-Z-VP isomer (i.e., cis-vinylphosphate,

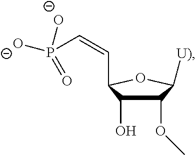

or mixtures thereof.

In another example, when the phosphate mimic is a 5′-vinyl phosphonate (VP), the 5′-terminal nucleotide can have the following structure,

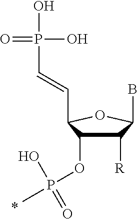

wherein * indicates the location of the bond to 5′-position of the adjacent nucleotide;
R is hydrogen, hydroxy, methoxy, or fluoro (e.g., hydroxy or methoxy), or another 2′-modification described herein; and
B is a nucleobase or a modified nucleobase, optionally where B is adenine, guanine, cytosine, thymine or uracil.

In some embodiments, when the phosphate mimic is a 5′-Vinyl phosphonate (VP), the 5′-terminal nucleotide may have the following structure,

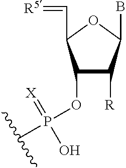

wherein X is O or S;
R is hydrogen, hydroxy, fluoro, or C1-20 alkoxy (e.g., methoxy or n-hexadecyloxy);
R^{5′} is ═C(H)—P(O)(OH)₂ and the double bond between the C5′ carbon and R5′ is in the E or Z orientation (e.g., E orientation); and
B is a nucleobase or a modified nucleobase, optionally where B is adenine, guanine, cytosine, thymine, or uracil.

In certain embodiments, R is methoxy and X is S. In certain embodiments, R is methoxy, X is S, and the double bond between the C5′ carbon and R^{5′} is in the E orientation.

In one embodiment, the dsRNA agent comprises a phosphorus-containing group at the 5′-end of the sense strand. In one embodiment, the dsRNA agent comprises a phosphorus-containing group at the 5′-end of the antisense strand.

In one embodiment, the dsRNA agent comprises a 5′-P. In one embodiment, the dsRNA agent comprises a 5′-P in the antisense strand.

In one embodiment, the dsRNA agent comprises a 5′-PS. In one embodiment, the dsRNA agent comprises a 5′-PS in the antisense strand.

In one embodiment, the dsRNA agent comprises a 5′-VP. In one embodiment, the dsRNA agent comprises a 5′-VP in the antisense strand. In one embodiment, the dsRNA agent comprises a 5′-E-VP in the antisense strand. In one embodiment, the dsRNA agent comprises a 5′-Z-VP in the antisense strand.

In one embodiment, the dsRNA agent comprises a 5′-PS₂. In one embodiment, the dsRNA agent comprises a 5′-PS₂ in the antisense strand.

In one embodiment, the dsRNA agent comprises a 5′-PS₂. In one embodiment, the dsRNA agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, 100%, 95%, 90%, 85%, 80%, 75%, 70%, 65%, 60%, 55%, 50%, 45%, 40%, 35% or 30% of the dsRNA agent of the invention is modified. For example, when 50% of the dsRNA agent is modified, 50% of all nucleotides present in the dsRNA agent contain a modification as described herein.

In one embodiment, each of the sense and antisense strands of the dsRNA agent is independently modified with acyclic nucleotides, LNA, HNA, CeNA, 2′-methoxyethyl, 2′-O-methyl, 2′-O-allyl, 2′-C-allyl, 2′-deoxy, 2′-deoxy-2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In one embodiment, the dsRNA agent of Formula (I) further comprises 3′ and/or 5′ overhang(s) of 1-10 nucleotides in length. In one example, dsRNA agent of formula (I) comprises a 3′ overhang at the 3′-end of the antisense strand and a blunt end at the 5′-end of the antisense strand. In another example, the dsRNA agent has a 5′ overhang at the 5′-end of the sense strand.

In one embodiment, the dsRNA agent of the invention does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent of the invention is 100% complementary to a target RNA to hybridize thereto and inhibits its expression through RNA interference. In another embodiment, the antisense strand of the dsRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the dsRNA represented by formula (I) can also apply to the dsRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

For example, the dsRNA agent as defined herein can comprise i) a phosphorus-containing group at the 5′-end of the sense strand or antisense strand; and ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand. In another embodiment, the targeting ligand is a lipophilic moiety and is conjugated according to one of the examples of Table 1

TABLE 1

| | Exemplary Conjugation Methods |
|---|---|
| 1 | Conjugated to one or more internal positions on at least one strand. |
| 2 | Conjugated to one or more internal positions on at least one strand, but excluding the terminal two positions from |
| | each end of the strand (e.g., excluding 4 positions: positions 1 and 2 counting from the 3′ end and positions 1 and 2 |
| | counting from the 5′ end). |
| 3 | Conjugated to one or more internal positions on at least one strand, but excluding the terminal three positions from |
| | each end of the strand (e.g., excluding 6 positions: positions 1, 2, and 3 counting from the 3′ end and positions 1, 2, |
| | and 3 counting from the 5′ end). |
| 4 | Conjugated to one or more internal positions on at least one strand, but excluding the cleavage site region of the |
| | sense strand. |
| 5 | Conjugated to one or more internal positions on at least one strand, but excluding positions 9-12 counting from the |
| | 5′-end of the sense strand and positions 11-13 counting from the 3′-end of the sense strand. |
| 6 | Conjugated to one or more internal positions on at least one strand, but excluding the cleavage site region of the |
| | antisense strand. |
| 7 | Conjugated to one or more internal positions on at least one strand, but excluding positions 12-14 counting from the |
| | 5′-end of the antisense strand. |
| 8 | Conjugated to one or more internal positions on at least one strand, which exclude positions 11-13 on the sense |
| | strand, counting from the 3′-end, and positions 12-14 on the antisense strand, counting from the 5′-end. |
| 9 | Conjugated to one or more of the following internal positions: positions 4-8 and 13-18 on the sense strand, and |
| | positions 6-10 and 15-18 on the antisense strand, counting from the 5′end of each strand. |
| 10 | Conjugated to one or more of the following internal positions: positions 5, 6, 7, 15, and 17 on the sense strand, and |
| | positions 15 and 17 on the antisense strand, counting from the 5′end of each strand. |
| 11 | Conjugated to one or more of the following internal positions: positions 4-8 and 13-18 on the sense strand, counting |
| | from the 5′end of the strand. |
| 12 | Conjugated to one or more of the following internal positions: positions 5, 6, 7, 15, and 17 (e.g., position 6) on the |
| | sense strand, counting from the 5′end of the strand. |
| 13 | Conjugated to one or more of the following internal positions: positions 6-10 and 15-18 on the antisense strand, |
| | counting from the 5′end of the strand. |
| 14 | Conjugated to one or more of the following internal positions: positions 15 and 17 on the antisense strand, counting |
| | from the 5′end of each strand. |

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof), and a targeting ligand. In one embodiment, the 5′-VP is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P and a targeting ligand. In one embodiment, the 5′-P is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS and a targeting ligand. In one embodiment, the 5′-PS is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS₂ and a targeting ligand. In one embodiment, the 5′-PS₂ is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end of the sense strand. In another embodiment, the targeting ligand is a lipophilic moiety and is conjugated according to one of the examples of Table 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, the dsRNA agents of the present invention comprise:
(a) a sense strand having:
(i) a length of 21 nucleotides;
(ii) a lipophilic moiety conjugated to one or more of (e.g., one of) the following internal positions: positions 4-8 and 13-18 on the sense strand, counting from the 5′-end of the sense strand; and
(iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense strand having:
(i) a length of 23 nucleotides;
(ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
(iii) phosphorothioate internucleotide linkages between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
(i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-OMe modifications at positions 1 to 6, 8, 10, 12, 14, and 16 to 21, and 2′-F modifications at positions 7, 9, 11, 13, and 15; and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-OMe modifications at positions 1 to 9, and 12 to 21, and 2′-F modifications at positions 10, and 11; and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-F modifications at positions 1, 3, 5, 7, 9 to 11, and 13, and 2′-OMe modifications at positions 2, 4, 6, 8, 12, and 14 to 21; and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
(iv) phosphorothioate internucleotide linkages between nucleotide positions 1 and 2, and between nucleotide positions 2 and 3 (counting from the 5′ end);
and
(b) an antisense strand having:
(i) a length of 25 nucleotides;
(ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a four nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand, counting from the 5′-end of the sense strand;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
(ii) a lipophilic moiety conjugated to one or more of (e.g., one of) the following internal positions: positions 4-8 and 13-18 on the sense strand;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
(b) an antisense strand having:
(i) a length of 23 nucleotides;
(ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 19 nucleotides;
(ii) a lipophilic moiety conjugated to one or more of (e.g., one of) the following internal positions: positions 4-8 and 13-18 on the sense strand;
(iii) 2′-OMe modifications at positions 1 to 4, 6, and 10 to 19, and 2′-F modifications at positions 5, and 7 to 9; and
(iv) phosphorothioate internucleotide linkages between nucleotide positions 1 and 2, and between nucleotide positions 2 and 3 (counting from the 5′ end);
and
(b) an antisense strand having:
(i) a length of 21 nucleotides;
(ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the dsRNA agents have a two nucleotide overhang at the 3′-end of the antisense strand, and a blunt end at the 5′-end of the antisense strand.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the iRNA agent of the invention is modified.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-deoxy-2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the double-stranded iRNA agent of the invention of the invention does not contain any 2′-F modification.

In some embodiments, the double-stranded iRNA agent of the invention contains one, two, three, four, five, six, seven, eight, nine, ten, eleven or twelve 2′-F modification(s). In one example, double-stranded iRNA agent of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent of the invention is 100% complementary to a target RNA to hybridize thereto and inhibits its expression through RNA interference. In another embodiment, the antisense strand of the iRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to an iRNA agent capable of inhibiting the expression of a target gene. The iRNA agent comprises a sense strand and an antisense strand, each strand having 14 to 40 nucleotides. The sense strand contains at least one thermally destabilizing nucleotide, wherein at least one said thermally destabilizing nucleotide occurs at or near the site that is opposite to the seed region of the antisense strand (i.e. at position 2-8 of the 5′-end of the antisense strand). For example, the thermally destabilizing nucleotide occurs between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that is smaller than a sterically demanding 2′-OMe are separated by 11 nucleotides in length. For example, the two modified nucleic acids are at positions 2 and 14 of the 5′end of the antisense strand.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

In some embodiments, the compound of the invention disclosed herein is an antimir. In some embodiments, compound of the invention comprises at least two antimirs covalently linked to each other via a nucleotide-based or non-nucleotide-based linker, for example a linker described in the disclosure, or non-covalently linked to each other. The terms “antimir” “microRNA inhibitor” or “miR inhibitor” are synonymous and refer to oligonucleotides or modified oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

In some embodiments, compound of the invention disclosed herein is an antagomir. In some embodiments, the compound of the invention comprises at least two antagomirs covalently linked to each other via a nucleotide-based or non-nucleotide-based linker, for example a linker described in the disclosure, or non-covalently linked to each other. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In a preferred embodiment, antagomir comprises a 2′-O-methyl modification at all nucleotides, a cholesterol moiety at 3′-end, two phosphorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Recent studies have found that dsRNA can also activate gene expression, a mechanism that has been termed “small RNA-induced gene activation” or RNAa (activating RNA). See for example Li, L. C. et al. Proc Natl Acad Sci USA. (2006), 103(46):17337-42 and Li L. C. (2008). “Small RNA-Mediated Gene Activation”. RNA and the Regulation of Gene Expression: A Hidden Layer of Complexity. Caister Academic Press. ISBN 978-1-904455-25-7. It has been shown that dsRNAs targeting gene promoters induce potent transcriptional activation of associated genes. Endogenous miRNA that cause RNAa has also been found in humans. Check E. Nature (2007). 448 (7156): 855-858.

Another surprising observation is that gene activation by RNAa is long-lasting. Induction of gene expression has been seen to last for over ten days. The prolonged effect of RNAa could be attributed to epigenetic changes at dsRNA target sites. In some embodiments, the RNA activator can increase the expression of a gene. In some embodiments, increased gene expression inhibits viability, growth development, and/or reproduction.

Accordingly, in some embodiments compound of the invention disclosed herein is activating RNA. In some embodiments, the compound of the invention comprises at least two activating RNAs covalently linked to each other via a nucleotide-based or non-nucleotide-based linker, for example a linker described in the disclosure, or non-covalently linked to each other.

Accordingly, in some embodiments, compound of the invention disclosed herein is a triplex forming oligonucleotide (TFO). In some embodiments, the compound of the invention comprises at least two TFOs covalently linked to each other via a nucleotide-based or non-nucleotide-based linker, for example a linker described in the disclosure, or non-covalently linked to each other. Recent studies have shown that triplex forming oligonucleotides can be designed which can recognize and bind to polypurine/polypyrimidine regions in double-stranded helical DNA in a sequence-specific manner. These recognition rules are outline by Maher III, L. J., et al., Science (1989) vol. 245, pp 725-730; Moser, H. E., et al., Science (1987) vol. 238, pp 645-630; Beal, P. A., et al., Science (1992) vol. 251, pp 1360-1363; Conney, M., et al., Science (1988) vol. 241, pp 456-459 and Hogan, M. E., et al., EP Publication 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 1 12:487-94). In general, the triplex-forming oligonucleotide has the sequence correspondence:

oligo
3′-A GGT
duplex
5′-A G CT
duplex
3′-T C GA

However, it has been shown that the A-AT and G-GC triplets have the greatest triple helical stability (Reither and Jeltsch, BMC Biochem, 2002, Seρtl2, Epub). The same authors have demonstrated that TFOs designed according to the A-AT and G-GC rule do not form non-specific triplexes, indicating that the triplex formation is indeed sequence specific.

Thus for any given sequence a triplex forming sequence can be devised. Triplex-forming oligonucleotides preferably are at least 15, more preferably 25, still more preferably 30 or more nucleotides in length, up to 50 or 100 nucleotides.

Formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFGl and endogenous HPRT genes in mammalian cells (Vasquez et al., Nucl Acids Res. 1999; 27: 1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-I gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Pat. App. Nos. 2003 017068 and 2003 0096980 to Froehler et al, and 2002 0128218 and 2002 0123476 to Emanuele et al, and U.S. Pat. No. 5,721,138 to Lawn, contents of which are herein incorporated in their entireties.

### Nucleic Acid Modifications

In some embodiments, the double-stranded iRNA agent of the invention comprises at least one nucleic acid modification described herein. For example, at least one modification selected from the group consisting of modified internucleoside linkage, modified nucleobase, modified sugar, and any combinations thereof. Without limitations, such a modification can be present anywhere in the double-stranded iRNA agent of the invention. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### Nucleic Acid Modifications (Sugar)

Double-stranded iRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furanosyl is connected to the 4′ position by a linker selected independently from —[C(R1)(R2)]ₙ-, —[C(R1)(R2)]ₙ-O—, —[C(R1)(R2)]ₙ-N(R1)-, —[C(R1)(R2)]ₙ-N(R1)-O—, —[C(R1R2)]ₙ-O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O) H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued U.S. patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Double-stranded iRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. Double-stranded iRNA agent of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in α configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

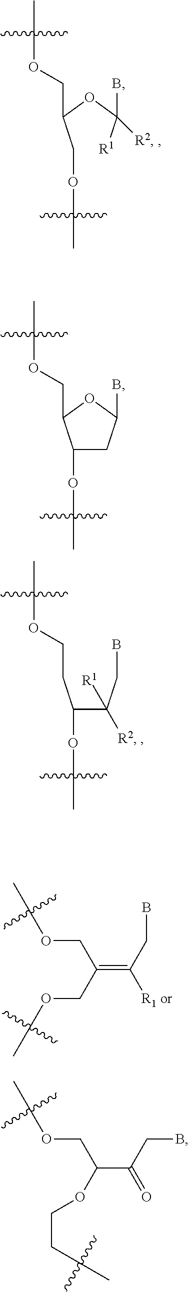

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

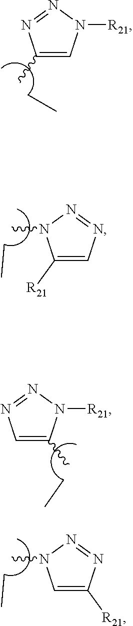

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alkyl metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

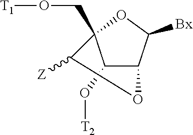

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C₁-C₆ alkyl, and X is O, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C₁-C₆ alkyl, or substituted C₁-C₆ alkyl and X is O or NJ1.

In certain embodiments, the Z group is C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is C₁-C₆ alkyl substituted with one or more Xx, wherein each Xx is independently halo (e.g., fluoro), hydroxyl, alkoxy (e.g., CH₃O—), substituted alkoxy or azido.

In certain embodiments, the Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is —CH₂Xx, wherein Xx is halo (e.g., fluoro), hydroxyl, alkoxy (e.g., CH₃O—) or azido.

In certain such embodiments, the Z group is in the (R)-configuration:

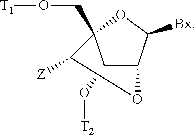

In certain such embodiments, the Z group is in the (S)-configuration:

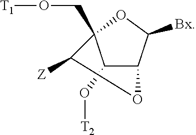

In certain embodiments, each T₁ and T₂ is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl)xanthine-9-yl (MOX). In certain embodiments, T₁ is a hydroxyl protecting group selected from acetyl, benzyl, t-butyldimethylsilyl, t-butyldiphenylsilyl and dimethoxytrityl wherein a more preferred hydroxyl protecting group is T₁ is 4,4′-dimethoxytrityl.

In certain embodiments, T₂ is a reactive phosphorus group wherein preferred reactive phosphorus groups include diisopropylcyanoethoxy phosphoramidite and H-phosphonate. In certain embodiments T₁ is 4,4′-dimethoxytrityl and T₂ is diisopropylcyanoethoxy phosphoramidite.

In certain embodiments, the compounds of the invention comprise at least one monomer of the formula:

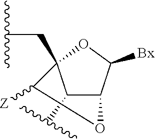

or of the formula:

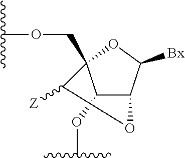

or of the formula:

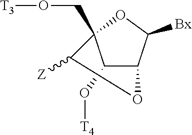

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In some embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C₁-C₆ alkyl or substituted C₁-C₆ alkyl. In certain embodiments, each Z is, independently, C₁-C₆ alkyl or substituted C₁-C₆ alkyl. In certain embodiments, at least one Z is C₁-C₆ alkyl. In certain embodiments, each Z is, independently, C₁-C₆ alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C₁-C₆ alkyl. In certain embodiments, each Z is, independently, substituted C₁-C₆ alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C₁-C₆ alkoxy (e.g., at least one Z is C₁-C₆ alkyl substituted with one or more C₁-C₆ alkoxy). In another embodiment, each substituent group is, independently, C₁-C₆ alkoxy (e.g., each Z is, independently, C₁-C₆ alkyl substituted with one or more C₁-C₆ alkoxy).

In certain embodiments, at least one C₁-C₆ alkoxy substituent group is CH₃O— (e.g., at least one Z is CH₃OCH₂—). In another embodiment, each C₁-C₆ alkoxy substituent group is CH₃O— (e.g., each Z is CH₃OCH₂—).

In certain embodiments, at least one substituent group is halogen (e.g., at least one Z is C₁-C₆ alkyl substituted with one or more halogen). In certain embodiments, each substituent group is, independently, halogen (e.g., each Z is, independently, C₁-C₆ alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C₁-C₆ alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C₁-C₆ alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, at least one Z group is C₁-C₆ alkyl substituted with one or more Xx, wherein each Xx is, independently, halo (e.g., fluoro), hydroxyl, alkoxy (e.g., CH₃O—) or azido.

In certain embodiments, each Z group is, independently, C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, C₁-C₆ alkyl substituted with one or more Xx, wherein each Xx is independently halo (e.g., fluoro), hydroxyl, alkoxy (e.g., CH₃O—) or azido.

In certain embodiments, at least one Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1 In certain embodiments, at least one Z group is —CH₂Xx, wherein Xx is halo (e.g., fluoro), hydroxyl, alkoxy (e.g., CH₃O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃—.

In certain embodiments, the Z group of at least one monomer is in the (R)-configuration represented by the formula:

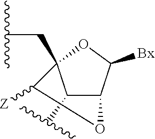

or the formula:

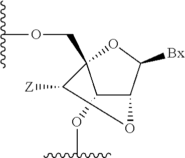

or the formula:

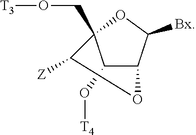

IN certain embodiments, the Z group of each monomer of the formula is in the (R)-configuration.

In certain embodiments, the Z group of at least one monomer is in the (S)-configuration represented by the formula:

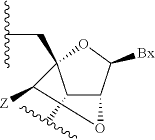

or the formula:

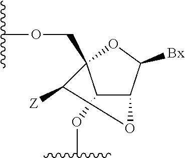

or the formula:

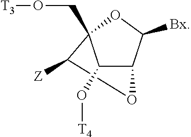

In certain embodiments, the Z group of each monomer of the formula is in the (S)-configuration.

In certain embodiments, T₃ is H or a hydroxyl protecting group. In certain embodiments, T₄ is H or a hydroxyl protecting group. In a further embodiment T₃ is an internucleoside linking group attached to a nucleoside, a nucleotide or a monomeric subunit. In certain embodiments, T₄ is an internucleoside linking group attached to a nucleoside, a nucleotide or a monomeric subunit. In certain embodiments, T₃ is an internucleoside linking group attached to an oligonucleoside or an oligonucleotide. In certain embodiments, T₄ is an internucleoside linking group attached to an oligonucleoside or an oligonucleotide. In certain embodiments, T₃ is an internucleoside linking group attached to an oligomeric compound. In certain embodiments, T₄ is an internucleoside linking group attached to an oligomeric compound. In certain embodiments, at least one of T₃ and T₄ comprises an internucleoside linking group selected from phosphodiester or phosphorothioate.

In certain embodiments, double-stranded iRNA agent of the invention comprise at least one region of at least two contiguous monomers of the formula:

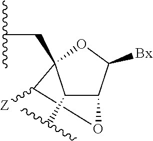

or of the formula:

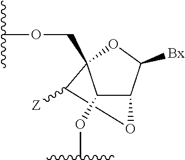

or of the formula:

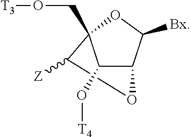

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below:

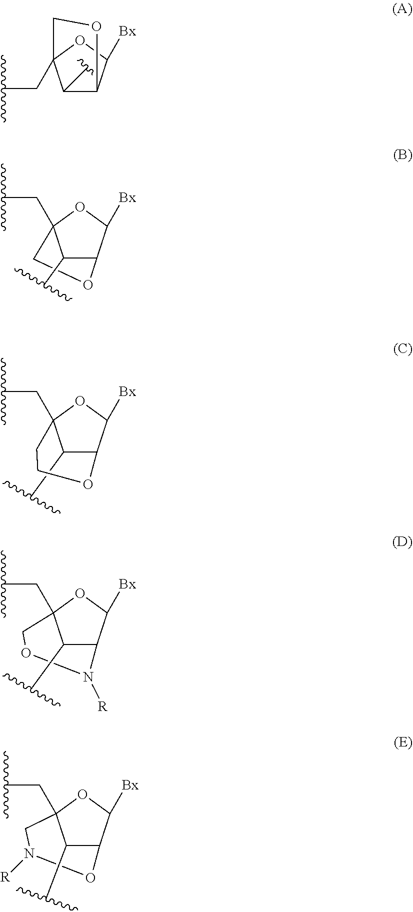

In certain embodiments, the double-stranded iRNA agent of the invention comprises at least two regions of at least two contiguous monomers of the above formula. In certain embodiments, the double-stranded iRNA agent of the invention comprises a gapped motif In certain embodiments, the double-stranded iRNA agent of the invention comprises at least one region of from about 8 to about 14 contiguous β-D-2′-deoxyribofuranosyl nucleosides. In certain embodiments, the Double-stranded iRNA agent of the invention comprises at least one region of from about 9 to about 12 contiguous β-D-2′-deoxyribofuranosyl nucleosides.

In certain embodiments, the double-stranded iRNA agent of the invention comprises at least one (e.g., 1, 2, 3, 4, 5, 6, 7, 8, 9, 10, 11, 12, 13, 14, 15 or more) comprises at least one (S)-cEt monomer of the formula:

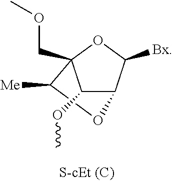

wherein Bx IS heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc.), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NIP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phosphotriesters, aminoalkylphosphotriesters, alkyl-phosphonaters (e.g., methyl-phosphonate), selenophosphates, phosphoramidates (e.g., N-alkylphosphoramidate), and boranophosphonates.

In some embodiments, the double-stranded iRNA agent of the invention comprises at least one (e.g., 1, 2, 3, 4, 5, 6, 7, 8, 9, 10, 11, 12, 13, 14, 15 or more and up to including all) modified or nonphosphodiester linkages. In some embodiments, the double-stranded iRNA agent of the invention comprises at least one (e.g., 1, 2, 3, 4, 5, 6, 7, 8, 9, 10, 11, 12, 13, 14, 15 or more and up to including all) phosphorothioate linkages.

The double-stranded iRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backbone-extended pyrrolidine PNA (bepPNA) nucleoside surrogates. A preferred surrogate is a PNA surrogate.

The double-stranded iRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the double-stranded iRNA agent of the inventions provided herein are all such possible isomers, as well as their racemic and optically pure forms.

### Nucleic Acid Modifications (Terminal Modifications

In some embodiments, the double-stranded iRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the double-stranded iRNA agent does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

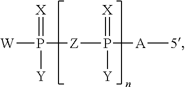

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc.), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W and Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc.), 5′-alkyletherphosphonates (R(OH)(O)P—O-5′, R=alkylether, e.g., methoxymethyl (CH₂OMe), ethoxymethyl, etc.). Other exemplary 5′-modifications include where Z is optionally substituted alkyl at least once, e.g., ((HO)₂(X)P—O[—(CH₂)ₐ—O—P(X)(OH)—O]_{b}— 5′, ((HO)₂(X)P—O[—(CH₂)ₐ—P(X)(OH)—O]_{b}— 5′, ((HO)2(X)P—[—(CH₂)ₐ—O—P(X)(OH)—O]_{b}— 5′; dialkyl terminal phosphates and phosphate mimics: HO[—(CH₂)ₐ—O—P(X)(OH)—O]_{b}— 5′, H₂N[—(CH₂)ₐ—O—P(X)(OH)—O]_{b}— 5′, H[—(CH₂)ₐ—O—P(X)(OH)—O]_{b}— 5′, Me₂N[—(CH₂)ₐ—O—P(X)(OH)—O]_{b}— 5′, HO[—(CH₂)ₐ—P(X)(OH)—O]_{b}— 5′, H₂N[—(CH₂)ₐ—P(X)(OH)—O]_{b}— 5′, H[—(CH₂)ₐ—P(X)(OH)—O]_{b}— 5′, Me₂N[—(CH₂)ₐ—P(X)(OH)—O]_{b}— 5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include, but are not limited to, abasic modification; mismatch with the opposing nucleotide in the opposing strand; and sugar modification such as 2′-deoxy modification, acyclic nucleotide, e.g., unlocked nucleic acids (UNA) or glycol nucleic acid (GNA); and 2′-5′-linked ribonucleotides (“3′-RNA”).

Exemplified abasic modifications are:

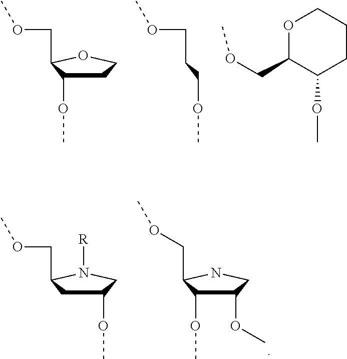

Exemplified sugar modifications are:

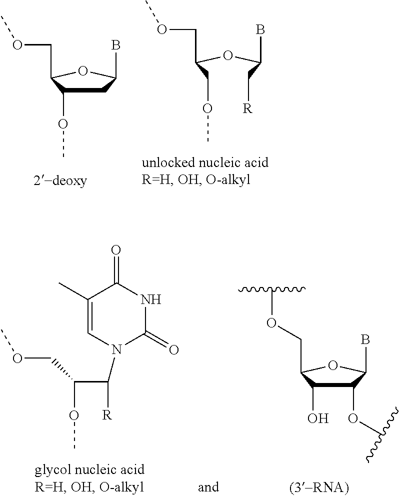

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

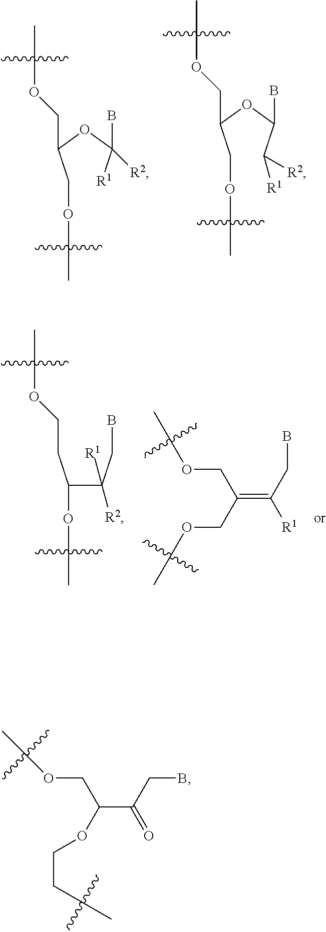

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

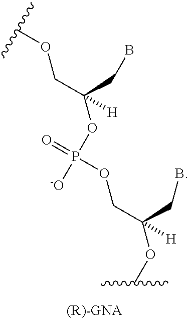

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

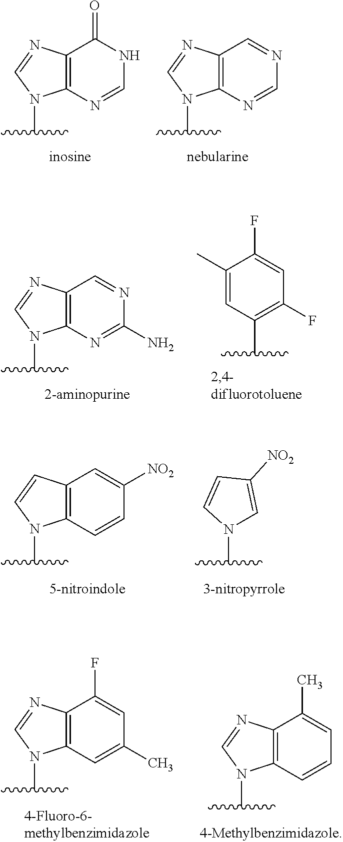

Exemplary phosphate modifications known to decrease the thermal stability of dsRNA duplexes compared to natural phosphodiester linkages are:

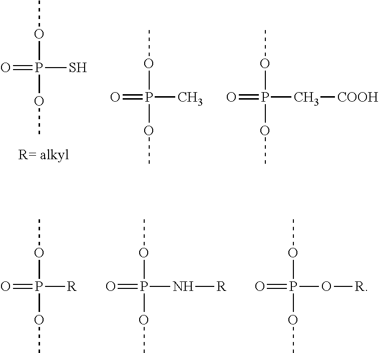

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment the iRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In some embodiments, at least one strand of the iRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for siRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the siRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLI1 fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Varicella zoster virus gene, a gene that is required for Varicella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in diploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a double-stranded iRNA agent of the invention that modulates a micro-RNA.

### Targeting CNS

In some embodiments, the invention provides a double-stranded iRNA agent that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, the invention provides a double-stranded iRNA agent that targets TARDBP for ALS, MAPT (Tau) for Frontotemporal Dementia, and HTT for Huntington Disease.

In some embodiments, the invention provides a double-stranded iRNA agent that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1 (CNS and Skeletal Muscle), GPR75 for obesity, LRRK2 for Parkinson disease, SARM1 for axial degeneration diseases (e.g., MS, ALS, and Parkinson's), and RPS25 for C9orf72 ALS/FTD and Huntington Disease, (e.g., Huntington-Like Syndrome Due To C9orf72 Expansions).

Dominant Inherited Spinocerebellar Ataxias, SCA1-8, are devastating disorders with no disease-modifying therapy. Exemplary targets include SCA2, SCA3, and SCA1.

### Targeting ATXN2 for SCA2

Spinocerebellar Ataxia 2 is the second most common SCA.
Disease: Spinocerebellar ataxia 2 (SCA2), a progressive ataxia; Amyotrophic lateral sclerosis (ALS)
Medical Need: Debilitating and ultimately lethal disease with no disease-modifying therapy
Prevalence: SCA is 2-6 per 100,000; ATXN2 causes 15% of SCA WW, much more in some countries, especially Cuba (40 per 100,000)
Target Validation: Excellent via human molecular genetics; coding CAG repeat expansion in ATXN2 discovered in familial and sporadic SCA and ALS
Target tissue: Spinal cord, brainstem, cerebellum
Mechanism: Autosomal dominant coding CAG expansion of ATXN2 causes expression of toxic, misfolded protein and Purkinje cell and neuronal death
Efficacy: 70% KD of ATXN2 mRNA; mATXN2 mice KD POC demonstrated
Safety: mATXN2 KO mice healthy
Diagnosis: Family history; genetic testing; early symptoms
Biomarkers: CSF CAG mRNA and peptide repeat proteins

### Targeting ATXN3 for SCA3

Spinocerebellar Ataxia 3 is the most common SCA WW.
Disease: Spinal cerebellar ataxia 3 (SCA3), a progressive ataxia
Medical Need: Debilitating and ultimately lethal disease with no disease-modifying therapy
Prevalence: Most common cause of SCA; SCA is 2-6 per 100,000; ATXN3 causes 21% of SCA in the U.S. and much more in Europe, especially Portugal
Target Validation: Excellent via human molecular genetics; coding CAG repeat expansion in ATXN3 discovered in familial and sporadic SCA
Target tissue: Spinal cord, brainstem, cerebellum
Mechanism: Autosomal dominant coding CAG expansion of ATXN3 causes expression of toxic, misfolded protein, Purkinje cell and neuron death
Efficacy: 70% KD of ATXN3 mRNA; mATXN3 KD mice POC demonstrated
Safety: mATXN3 KO mice healthy
Diagnosis: Family history; genetic testing; early symptoms
Biomarkers: CSF CAG mRNA and peptide repeat proteins

### Targeting ATXN1 for SCA1

Spinocerebellar Ataxia 1 is the first SCA gene discovered in 1993.
Disease: Spinocerebellar ataxia 1 (SCA1), a progressive ataxia
Medical Need: Debilitating and ultimately lethal disease with no disease-modifying therapy
Prevalence: SCA is 2-6 per 100,000; ATXN1 causes 6% of SCA in the U.S. and WW, much more in some countries (25% Japan), especially Poland (64%) and Siberia (100%)
Target Validation: Excellent via human molecular genetics; coding CAG repeat expansion in ATXN1 discovered in familial and sporadic SCA
Target tissue: Spinal cord, brainstem, cerebellum
Mechanism: Autosomal dominant coding CAG expansion of ATXN1 causes expression of toxic, misfolded protein, Purkinje cell and neuronal death
Efficacy: 70% KD of ATXN1 mRNA; mATXN1 mice POC demonstrated
Safety: mATXN1 KO mice healthy
Diagnosis: Family history; genetic testing; early symptoms
Biomarkers: CSF CAG mRNA and peptide repeat proteins

### Targeting ATXN7 for SCA7

Spinocerebellar Ataxia 7 causes Progressive Ataxia and Retinal Degeneration
Disease: Spinocerebellar ataxia 7 (SCA7), a progressive ataxia with blindness
Medical Need: Debilitating and ultimately lethal retinal and cerebellar disorder with no disease-modifying therapy
Prevalence: SCA is 2-6 per 100,000; ATXN7 causes 5% of SCA WW, much more in some countries, especially South Africa
Target Validation: Excellent via human molecular genetics; coding CAG repeat expansion in ATXN7 discovered in familial and sporadic SCA
Target tissue: Spinal cord, brainstem, cerebellum and retina
Mechanism: Autosomal dominant coding CAG expansion of ATXN1 causes expression of toxic, misfolded protein, inciting cone and rod dystrophy, Purkinje cell and neuronal lethality
Efficacy: 70% KD of ATXN1 mRNA; IT AND IVT
Safety: No report of ATXN7 KO mice found yet
Diagnosis: Family history; genetic testing; early symptoms
Biomarkers: CSF CAG mRNA and peptide repeat proteins

### Targeting ATXN8 for SCA8

Spinocerebellar Ataxia 8 is caused by CTG repeat expansion in ATXN8.
Disease: Spinocerebellar ataxia 8 (SCA8), a progressive neurodegenerative ataxia
Medical Need: Debilitating and ultimately lethal disease with no disease-modifying therapy
Prevalence: SCA is 2-6 per 100,000; ATXN8 causes 3% of SCA WW, much more in some countries, especially Finland
Target Validation: Excellent via human molecular genetics; coding CTG repeat expansion in ATXN8 discovered in familial and sporadic SCA
Target tissue: Spinal cord, brainstem, cerebellum
Mechanism: Autosomal dominant coding CTG expansion of ATXN8 causes expression of toxic, misfolded protein, inciting Purkinje cell and neuronal lethality
Efficacy: 70% KD of ATXN8 mRNA
Safety: No ATXN8 KD mice reported yet
Diagnosis: Family history; genetic testing; early symptoms
Biomarkers: CSF CTG mRNA and peptide repeat proteins

### Targeting CACNA1A for SCA6

Androgen receptor mutations causes SBMA and other diseases.
Disease: Spinal and bulbar muscular atrophy (SBMA, Kennedy disease), a progressive muscle wasting disease
Medical Need: Debilitating and ultimately lethal disease with no disease-modifying therapy
Prevalence: SBMA is 2 per 100,000 males; Females have a mild phenotype
Target Validation: Excellent via human molecular genetics; coding CAG repeat expansion in AR discovered in familial SBMA
Target tissue: Spinal cord, brainstem
Mechanism: X-linked coding CAG expansion of AR causes toxic gain-or-function and motor neuron lethality
Efficacy: 70% KD of AR
Safety: AR LOF causes testicular feminization syndrome
Diagnosis: Family history; genetic testing; early symptoms
Biomarkers: CSF CAG mRNA and peptide repeat proteins

Inherited Polyglutamine Disorders. Exemplary target includes HD.

### Targeting HTT for Huntington Disease

Huntington mutations causes HD.
Disease: Huntington disease (HD), a progressive CNS degenerative disease
Medical Need: Debilitating and ultimately lethal disease with no disease-modifying therapy
Prevalence: HD is 5-10 per 100,000 WW; much more common is certain countries, especially Venezuela
Target Validation: Excellent via human molecular genetics; coding CAG repeat expansion in HTT discovered in familial and sporadic HD
Target tissue: Striatum, cortex
Mechanism: Autosomal dominant coding CAG expansion of HTT causes expression of toxic, misfolded protein and neuronal death
Efficacy: 70% KD of HTT CAG expansion only; murine POC demonstrated
Safety: KO of HTT in mice is lethal; KD in humans demonstrated
Diagnosis: Family history; genetic testing; early symptoms
Biomarkers: CSF mRNA and peptide repeat proteins

### Targeting ATN1 for DRPLA

Atrophin 1 mutations causes DRPLA.
Disease: Dentatorubral-pallidoluysian atrophy (DRPLA), a progressive spinocerebellar disorder similar to HD
Medical Need: Debilitating and ultimately lethal disease with no disease-modifying therapy
Prevalence: DRPLA is 2-7 per 1,000,000 in Japan
Target Validation: Excellent via human molecular genetics; coding CAG repeat expansion in ATN1 discovered in familial and sporadic SCA
Target tissue: Spinal cord, brainstem, cerebellum and cortex
Mechanism: Autosomal dominant coding CAG expansion of ATN1 causes expression of toxic, misfolded protein and neuronal death
Efficacy: 70% KD of ATN1
Safety: ATN1 KO mice ae healthy
Diagnosis: Family history; genetic testing; early symptoms
Biomarkers: CSF CAG mRNA and peptide repeat proteins

### Targeting AR for Spinal and Bulbar Muscular Atrophy

Androgen receptor mutations causes SBMA and other diseases.
Disease: Spinal and bulbar muscular atrophy (SBMA, Kennedy disease), a progressive muscle wasting disease
Medical Need: Debilitating and ultimately lethal disease with no disease-modifying therapy
Prevalence: SBMA is 2 per 100,000 males; Females have a mild phenotype
Target Validation: Excellent via human molecular genetics; coding CAG repeat expansion in AR discovered in familial SBMA
Target tissue: Spinal cord, brainstem
Mechanism: X-linked coding CAG expansion of AR causes toxic gain-or-function and motor neuron lethality
Efficacy: 70% KD of AR
Safety: AR LOF causes testicular feminization syndrome
Diagnosis: Family history; genetic testing; early symptoms
Biomarkers: CSF CAG mRNA and peptide repeat proteins

### Targeting FXN for Friedrich Ataxia

Recessive LOF GAA expansion of FXN causes FA.
Disease: Friedrich ataxia (FA), a progressive degenerative ataxia
Medical Need: Debilitating and ultimately lethal disease with no disease-modifying therapy
Prevalence: FA is 2 per 100,000 WW
Target Validation: Excellent via human molecular genetics; intron GAA repeat expansion in FXN discovered in familial FA
Target tissue: Spinal cord and cerebellum; may also affect retina and heart
Mechanism: Autosomal recessive non-coding FAA expansion of FXN causes deceased expression of FXN, an important mitochondrial protein
Efficacy: 70% KD of FXN intron GAS expansion
Safety: KD of intron GAA is safe and effective in mice
Diagnosis: Family history; genetic testing; early symptoms
Biomarkers: CSF mRNA and peptide repeat proteins

### Targeting FMR1 for FXTAS

Fragile X-associated tremor/ataxia syndrome caused by FMR1 overexpression.
Disease: Fragile X-associated tremor/ataxia syndrome (FXTAS), a progressive disorder of ataxia and cognitive loss in adults
Medical Need: Debilitating disease with no disease-modifying therapy
Prevalence: FMR1 permutation is 1 in 500 males
Target Validation: Excellent via human molecular genetics; coding CCG repeat expansion pre-mutations in FMR1 discovered in FXTAS
Target tissue: spinal cord, cerebellum, cortex
Mechanism: X-linked coding CCG expansion of FMR1 causes toxic mRNA
Efficacy: 70% KD of toxic mRNA
Safety: LOF toxicity
Diagnosis: Family history; genetic testing; early symptoms
Biomarkers: CSF mRNA and peptide repeat proteins

### Targeting Upstream of FMR1 for Fragile X Syndrome

Target upstream mRNA of FMR1 to treat FRAXA
Disease: Fragile X syndrome (FRAXA), a progressive disorder of mental retardation
Medical Need: Debilitating disease with no disease-modifying therapy
Prevalence: FRAXA is 1 per 4,000 males and 1 per 8,000 females
Target Validation: Excellent via human molecular genetics; coding CCG repeat expansion in FMR1 discovered in FRAXA
Target tissue: CNS
Mechanism: X-linked coding CCG expansion of FMR1 causes LOF; NormalFMR1 functions to transport specific mRNAs from nucleus
Efficacy: 70% KD of toxic mRNA
Safety: Need to define specific targets
Diagnosis: Family history; genetic testing; early symptoms
Biomarkers: CSF mRNA and peptide repeat proteins

Dominant Inherited Amyotrophic Lateral Sclerosis is a devastating disorders with no disease-modifying therapy. Exemplary targets include C9orf72, ATXN2 (also causes SCA2), and MAPT.

### Targeting C9orf72 for ALS

C9orf72 is the most common cause of ALS.
Disease: Amyotrophic Lateral Sclerosis (ALS) and Frontotemporal Dementia (FTD)
Medical Need: Lethal disorder of motor neurons with no disease-modifying therapy
Prevalence: Most common cause of ALS; ALS is 2-5 per 100,000 (10% is familial); C9orf72 causes 39% of familial ALS in the U.S. and Europe and 7% of sporadic ALS
Target Validation: Excellent via human molecular genetics; hexa-nucleotide expansion discovered in familial and sporadic ALS
Target tissue: Upper and lower motor neurons for ALS; Cortex for FTD
Mechanism: Autosomal dominant hexa-nucleotide expansion causes repeat-associated non-AUG-dependent translation of toxic dipeptide repeat proteins and neuron lethality
Efficacy: 70% KD of C9orf72
Safety: Heterozygous LOF mutations of C9orf72 appear safe in humans and mice
Diagnosis: Family history; genetic testing; early symptoms
Biomarkers: CSF hexa-nucleotide repeat mRNAs and dipeptide repeat proteins

### Targeting TARDBP for ALS

TARDBP mutations causes ALS and FTD
Disease: Amyotrophic Lateral Sclerosis (ALS) and Frontotemporal Dementia (FTD)
Medical Need: Lethal disorder of motor neurons with no disease-modifying therapy
Prevalence: ALS is 2-5 per 100,000 (10% is familial); TARDBP causes 5% of familial ALS and 1.5% of sporadic ALS
Target Validation: Excellent via human molecular genetics; mutations discovered in familial and sporadic ALS
Target tissue: Upper and lower motor neurons for ALS; Cortex for FTD
Mechanism: Autosomal dominant TRDBP mutations cause toxic TRDBP protein and neuron lethality
Efficacy: 70% KD of TARDBP mutant alleles
Safety: KO mice are embryonic lethal
Diagnosis: Family history; genetic testing; early symptoms
Biomarkers: CSF proteins

### Targeting FUS for ALS

FUS mutations causes ALS and FTD.
Disease: Amyotrophic Lateral Sclerosis (ALS)
Medical Need: Lethal disorder of motor neurons with no disease-modifying therapy
Prevalence: ALS is 2-5 per 100,000 (10% is familial); FUS causes 5% of familial ALS; FUS inclusions are often found in sporadic ALS
Target Validation: Excellent via human molecular genetics; mutations discovered in familial ALS
Target tissue: Upper and lower motor neurons for ALS
Mechanism: Autosomal dominant FUS mutations cause abnormal protein folding and neuron lethality
Efficacy: 70% KD of FUS mutant alleles
Safety: KO mice struggle but survive and have an ADHD phenotype
Diagnosis: Family history; genetic testing; early symptoms
Biomarkers: CSF proteins

### Targeting SOD1 for ALS

Dominant and recessive mutations of SOD1 cause ALS.
Disease: Amyotrophic Lateral Sclerosis (ALS)
Medical Need: Lethal disorder of motor neurons with no disease-modifying therapy
Prevalence: ALS is 2-5 per 100,000 (10% is familial); SOD1 causes 5-20% of familial ALS
Target Validation: Excellent via human molecular genetics; many SOD1 mutations associated with AD and AR ALS in families
Target tissue: Upper and lower motor neurons for ALS
Efficacy: Possibly need mutation-specific KD
Diagnosis: Family history; genetic testing; early symptoms
Biomarkers: Mutation-specific

Dominant Inherited Frontotemporal Dementia and Progressive Supra-nuclear Palsy. The targets include MAPT because it may be important for AD, or C9orf72.

### Targeting Microtubule-Associated Protein Tau for FTD-17 and PSP

Familial Frontotemporal Dementia 17 and Familial Progressive Supra-nuclear Palsy
Disease: Frontotemporal Dementia 17 (FTD-17), a familial form of FTD lined to chromosome 17; MAPT mutations also cause rare forms of Progressive Supra-nuclear Palsy, Corticobasal Degeneration, Tauopathy with Respiratory Failure, Dementia with Seizures
Medical Need: Lethal neurodegenerative disorder with no disease-modifying therapy
Prevalence: FTD is 15-22 per 100,000; FTD-17 WW prevalence unknown, but in Netherlands it is 1 in 1,000,000
Target Validation: Excellent via human molecular genetics; GOF point and splice site mutations of MAPT discovered in familial and sporadic FTD
Target tissue: Frontal and Temporal Cortex
Mechanism: Autosomal dominant GOF mutations of MAPT lead to toxic Tau peptides and neuronal death
Efficacy: 70% KD of MAPT may be sufficient
Safety: MAPT KO mice healthy
Diagnosis: Family history; genetic testing; early symptoms
Biomarkers: CSF Tau mRNAs and proteins

### Targeting Sequestosome 1 for FTD and ALS

Sporadic FTD/ALS associated with dominant SQSTM1 mutations
Disease: Frontotemporal Dementia and/or ALS
Medical Need: Lethal neurodegenerative disorder with no disease-modifying therapy
Prevalence: Very rare
Target Validation: Reasonable via human molecular genetic association in sporadic cases
Target tissue: Frontal and Temporal Cortex, Cerebellum and Spinal Cord
Safety: Specific missense mutations associated with Paget disease; KO mice have hematologic disorder
Diagnosis: Genetic testing; early symptoms
Biomarkers: CSF possible

Dominant Inherited Parkinson Disease is a devastating disorders with no disease-modifying therapy. The targets include SNCA.

### Targeting SNCA for Parkinson Disease

Alpha Synuclein mutations causes familial Parkinson disease.
Disease: Parkinson disease (PD) and Lewy body dementia
Medical Need: Lethal neurodegenerative disorder with no disease-modifying therapy
Prevalence: 4 M WW; ⅓ of PD is familial; 1% of fPD caused by SNCA
Target Validation: Excellent via human molecular genetics; SNCA point mutations and duplications cause familial PD
Target tissue: Medulla oblongata; Substantia Nigra of the midbrain
Mechanism: Overexpression or expression of abnormal SNCA protein leads to toxic peptides and neuronal death
Efficacy: 70% KD of SNCA
Safety: SNCA KO mice are healthy
Diagnosis: Family history; genetic testing; early symptoms
Biomarkers: CSF SNCA mRNAs and proteins

### Targeting LRRK2 for Parkinson Disease

Leucine-rich repeat kinase 2 mutations cause familial Parkinson disease.
Disease: Parkinson disease (PD)
Medical Need: Lethal neurodegenerative disorder with no disease-modifying therapy
Prevalence: 4 M WW; ⅓ of PD is familial; 3-7% of fPD caused by LRRK2
Target Validation: Excellent via human molecular genetics; LRRK2 point mutations cause familial PD
Target tissue: Medulla oblongata; Substantia Nigra of the midbrain
Mechanism: Not clear if GOF or LOF mechanism
Diagnosis: Family history; genetic testing; early symptoms
Biomarkers: CSF mRNAs and proteins

### Targeting GARS for Spinal Muscular Atrophy V

Autosomal dominant Glycyl-tRNA Synthetase mutations causes SMAV.
Disease: Spinal muscular atrophy V (SMAV) or distal hereditary motor neuropathy Va
Medical Need: Neurodegenerative disorder with no disease-modifying therapy
Prevalence: Very rare
Target Validation: Good via human molecular genetics; GARS point mutations cause familial SMA
Target tissue: Spinal cod
Diagnosis: Family history; genetic testing; early symptoms

### Targeting Seipin for Spinal Muscular Atrophy

Autosomal dominant Seipin mutations causes SMA.
Disease: Spinal muscular atrophy (SMA) or distal hereditary motor neuropathy
Medical Need: Neurodegenerative disorder with no disease-modifying therapy
Prevalence: Very rare
Target Validation: Good via human molecular genetics; Seipin point mutations cause familial SMA
Target tissue: Spinal cod
Mechanism: Probably GOF and toxic peptides
Efficacy: 50% KD may be safe and effective
Safety: Recessive LOF mutations cause Progressive encephalopathy with or without lipodystrophy
Diagnosis: Family history; genetic testing; early symptoms

Dominant Inherited Alzheimer Disease is a devastating disorders with no disease-modifying therapy. The targets include APP because of central mechanistic role in familial disease and possible role in common AD.

### Targeting APP for Alzheimer Disease

Amyloid precursor protein mutations causes early onset familial Alzheimer disease.
Disease: Early Onset Familial Alzheimer Disease (EOFAD); AD in Down syndrome; AD
Medical Need: Lethal neurodegenerative disorder with no disease-modifying therapy
Prevalence: EOFAD-APP 1% AD; Trisomy 21 1% AD; AD 2.5-5 M in US
Target Validation: Excellent via human molecular genetics; APP duplications and point mutations cause EOFAD
Target tissue: Cerebral cortex; Initially Hippocampus
Mechanism: APP overexpression or expression of toxic metabolites cause progressive neuronal death
Efficacy: 70% KD of APP
Safety: KD mice healthy with some behavioral abnormalities; KD mice healthy with some spatial memory defects
Diagnosis: Family history; genetic testing; early symptoms; MRI
Biomarkers: CSF APP mRNA and peptides

### Targeting PSEN1 for Alzheimer Disease

Presenilin 1 mutations causes early onset familial Alzheimer disease.
Disease: Early Onset Familial Alzheimer Disease (EOFAD); AD
Medical Need: Lethal neurodegenerative disorder with no disease-modifying therapy
Prevalence: 70% of EOFAD
Target Validation: Excellent via human molecular genetics; PSEN1 point mutations cause EOFAD
Target tissue: Cerebral cortex; Initially Hippocampus
Mechanism: Autosomal dominant mutations of PSEN1 cause abnormal APP metabolism and toxic peptides cause progressive neuronal death
Efficacy: APP KD may obviate need for PSEN1-specific therapy
Safety: PSEN1 mutations associated with familial dilated cardiomyopathy and Hidradenitis suppuratibva (skin); PSEN1 critical for NOTCH signaling and development
Diagnosis: Family history; genetic testing; early symptoms; MRI
Biomarkers: CSF PSEN1 and APP peptides

### Targeting PSEN2 for Alzheimer Disease

Presenilin 2 mutations causes early onset familial Alzheimer disease.
Disease: Early Onset Familial Alzheimer Disease (EOFAD); AD
Medical Need: Lethal neurodegenerative disorder with no disease-modifying therapy
Prevalence: Rare
Target Validation: Excellent via human molecular genetics; PSEN2 point mutations cause EOFAD
Target tissue: Cerebral cortex; Initially Hippocampus
Mechanism: Autosomal dominant mutations of PSEN2 cause abnormal APP metabolism and toxic peptides cause progressive neuronal death
Efficacy: APP KD may obviate need for PSEN2-specific therapy
Safety: PSEN2 mutations associated with familial dilated cardiomyopathy; PSEN2 important for NOTCH signaling and development
Diagnosis: Family history; genetic testing; early symptoms; MRI
Biomarkers: CSF PSEN2 and APP peptides

### Targeting Apo E for Alzheimer Disease

Apolipoprotein E4 is associated with AD in the elderly.
Disease: Sporadic Alzheimer Disease in the elderly
Medical Need: Lethal neurodegenerative disorder with no disease-modifying therapy
Prevalence: AD 2.5-5 M in the U.S. and growing
Target Validation: Genomic evidence supporting the association between ApoE4 and AD is excellent in many populations. In Nigeria, however, the polymorphism is very common and AD is not. No familial human genetic studies demonstrate that Apo E4 homozygosity is sufficient to cause AD. In Framingham epidemiology studies, half of AD patients did not have an Apo E4 allele and most Apo E4 carriers did not develop dementia.
Target tissue: Cerebral cortex
Mechanism: It is not yet clear if Apo E4 contributes to the pathogenesis of AD despite the strong association in many populations. Thus far, data indicate that Apo E4 homozygosity indicates increased risk of Ad in the elderly but is not sufficient for causing AD, even in the elderly.
Safety: KD of Apo E in CNS may be safe as human LOF mutations in Apo E are not associated with obvious neurologic defects; Systemic exposure may cause hyperlipoproteinemia type III
Diagnosis: Clinical diagnosis of AD; Exclusion of EOFAD mutation; Genetic testing for the Apo E4 genotype
Biomarkers: CSF APP, Tau mRNA and peptides

CNS Gene Duplication Disorders. Consistent KD by half may ameliorate these disorders. The targets include MeCP2.

### Targeting MeCP2 for X-Linked Mental Retardation

Methyl CpG Binding Protein 2 gene duplication causes XLMR.
Disease: X-linked Mental Retardation
Medical Need: Lethal cognitive disorder with no disease-modifying therapy
Prevalence: 1-15% of X-linked MR caused by MeCP2 duplication; 2-3% of population has MR
Target Validation: Excellent via human molecular genetics; MeCP2 duplication causes XLMR
Target tissue: Cerebral cortex
Mechanism: MeCP2 over-expression cause dysregulation of other gene and neurodegeneration
Efficacy: 50% KD of MeCP2; ASO KD in mouse models reverse phenotype
Safety: MeCP2 LOF mutations cause Rett syndrome
Diagnosis: Family history; genetic testing; early symptoms
Biomarkers: CSF MeCP2 mRNA and peptides

### Targeting ITM2B for CAA

Integral Membrane Protein 2B mutations causes Familial British Dementia.
Disease: Cerebral Amyloid Angiopathy, British Type or FBD; Specific mutation may also cause dominant retinal degeneration
Medical Need: Lethal disorder with no disease-modifying therapy
Prevalence: Rare
Target Validation: Excellent via human molecular genetics
Target Tissue: CNS vascular system, CNS
Mechanism: probably GOF mutations
Efficacy: 70% KD of ITM2B mutant allele may be effective
Diagnosis: Family history; genetic testing; early symptoms
Biomarkers: CSF mRNA and protein possible

### Targeting CST3 for CAA

Cystatin C mutations causes familial cerebral amyloid angiopathy.
Disease: Cerebral Amyloid Angiopathy, Icelandic type
Medical Need: Lethal disorder with no disease-modifying therapy
Prevalence: Rare, except in Iceland and Denmark
Target Validation: Excellent via human genetics
Target Tissue: CNS vascular system
Mechanism: Mutant protein accumulates in vascular adventitia, causing CNS bleeds
Efficacy: Possibly 70% KD of mutant allele
Safety: CST3 KO mice may have risk of arthritis;
Diagnosis: Family history; genetic testing; early symptoms
Biomarkers: CSF mRNA and protein possible

### Targeting SPAST for Spastic Paraplegia

SPASTIN mutations causes Spastic Paraplegia 4 with cognitive loss.
Disease: Spastic paraplegia (SP) with cognitive loss
Medical Need: Lower motor neurodegenerative disorder with no disease-modifying therapy
Prevalence: SP is 5 per 100,000; SP4 is 45% of dominant SP
Target Validation: Excellent via human molecular genetics; SPAST trinucleotide mutations cases familial SP
Target tissue: Spinal cord; CNS
Mechanism: Nonsense and probable dominant-negative mutations cause abnormal microtubule metabolism and neurodegeneration
Efficacy: Probably need gene replacement
Diagnosis: Family history; genetic testing; early symptoms
Biomarkers: CSF SPAST mRNAs and proteins possible

### Targeting KIF5A for Spastic Paraplegia

Kinesin Family Member 5A mutations causes Spastic Paraplegia 10 and other disorders.
Disease: Spastic paraplegia (SP) with peripheral neuropathy
Medical Need: Lower motor neurodegenerative disorder with no disease-modifying therapy
Prevalence: SP is 5 per 100,000; SP10 is 1 per 1,000,000
Target Validation: Excellent via human molecular genetics; KIF5A amino terminal missense mutations cause SP10; KIF5A is expressed in the CNS and encodes a microtubule motor protein
Target tissue: Spinal cord
Mechanism: Autosomal dominant missense mutations cause SP10 possibly affect microtubule binding to the motor
Efficacy: Possibly KD of mutant alleles
Safety: KIF5A frameshift mutations cause Neonatal intractable myoclonus and splice site mutations are associated with familial ALS, possibly through LOF mechanisms
Diagnosis: Family history; genetic testing; early symptoms
Biomarkers: CSF mRNAs and proteins possible

### Targeting ATL1 for Spastic Paraplegia

Atlastin mutations causes Spastic Paraplegia 3A and Sensory Neuropathy 1D.
Disease: Spastic paraplegia (SP) and Hereditary Sensory Neuropathy (HSN)
Medical Need: Lower motor neurodegenerative disorder with no disease-modifying therapy
Prevalence: SP is 5 per 100,000; 3A is a rare dominant form
Target Validation: Excellent via human molecular genetics; ATL1 point mutations cause familial SP
Target tissue: Spinal cord
Mechanism: Autosomal dominant expression of dominant-negative ATL1 protein causes SP3A; However, LOF mutations causes Sensory Neuropathy 1D
Efficacy: 70% KD of specific ATL1 allele
Safety: ATL1 heterozygous LOF mutations causes HSN1D
Diagnosis: Family history; genetic testing; early symptoms
Biomarkers: CSF ATL1 mRNAs and proteins

### Targeting NIPA1 for Spastic Paraplegia

LOF NIPA1 mutations cause Spastic Paraplegia 6 with Seizures.
Disease: Spastic paraplegia (SP) with epilepsy
Medical Need: Lower motor neurodegenerative disorder with no disease-modifying therapy
Prevalence: SP is 5 per 100,000; SP6 is a rare dominant form
Target Validation: Excellent via human molecular genetics; NIPA1 point mutations cause familial SP
Target tissue: Spinal cord; CNS
Mechanism: Autosomal dominant expression of defective membrane protein causes SP3A; Possible LOF
Efficacy: Gene replacement may be required
Safety: Possible LOF mechanism
Diagnosis: Family history; genetic testing; early symptoms
Biomarkers: CSF mRNAs and proteins possible

Dominant Inherited Myotonic Dystrophy is a disorder of CNS, Skeletal Muscle and Cardiac Muscle Requiring CNS and Systemic Therapy. The targets include MPK for DM1.

### Targeting DMPK for Myotonic Dystrophy 1

Dystrophia Myotonica Protein Kinase: CNS and systemic therapy needed for effective therapy.
Disease: Myotonic dystrophy 1 (DM1), a degenerative disorder of muscle and CNS
Medical Need: Lethal disorder with no disease-modifying therapy
Prevalence: 1 per 8,000 WW
Target Validation: Excellent via human molecular genetics; DMPK CTG repeat expansion cases familial DM1
Target tissue: Skeletal muscle, cardiac muscle, CNS
Mechanism: Autosomal dominant non-coding CTG repeat causes abnormal RNA processing and dominant negative effect; Anticipation from extreme expansion causes early onset disease
Efficacy: 70% of DMPK; ASO efficacy demonstrated in mice
Safety: Demonstrated in mice with KO and ASO KD
Diagnosis: Family history; genetic testing; early symptoms
Biomarkers: Blood and CSF mRNAs and proteins

### Targeting ZNF9 for Myotonic Dystrophy 2

Zinc Finger Protein 9 mutations causes this skeletal muscle disorder.
Disease: Myotonic dystrophy 2 (DM2), a degenerative disorder of muscle
Medical Need: Serious disorder with no disease-modifying therapy
Prevalence: 1 per 8,000 WW; Most common muscular dystrophy in adults
Target Validation: Excellent via human molecular genetics; ZNF9 CTTG repeat expansion in intron 1 cases familial DM2
Target tissue: Skeletal muscle, cardiac muscle
Mechanism: Autosomal dominant CTTG repeat expansion in intron 1 causes abnormal RNA metabolism and dominant negative effects, No anticipation documented for DM2
Efficacy: 70% of ZNF9
Safety: Safe KD in mice demonstrated
Diagnosis: Family history; genetic testing; early symptoms
Biomarkers: Blood mRNAs and proteins

Dominant Inherited Prion Diseases are inherited, sporadic and transmissible PRNP disorders. The targets include PRNP.

### Targeting PRNP for Myotonic Prion Diseases

Zinc Finger Protein 9 mutations causes this skeletal muscle disorder.
Disease: Dominant inherited Prion diseases, including PRNP-Related Cerebral Amyloid Angiopathy, Gerstmann-Straussler Disease (GSD), Creutzfeldt-Jakob Disease (CJD), Fatal Familial Insomnia (FFI), Huntington Disease-Like 1 (HDL1), Kuru susceptibility
Medical Need: Lethal neurodegenerative disorders with no disease-modifying therapy
Prevalence: 1 per 1,000,000
Target Validation: Excellent via human molecular genetics; PRNP mutations causes familial and sporadic Prion disease
Target tissue: CNS
Mechanism: Autosomal dominant protein mid-folding causes neurotoxicity
Efficacy: 70% of PRNP KD; PRNP polymorphisms appear protective for Kuru
Safety: PRNP KO mice are healthy
Diagnosis: Family history; genetic testing; early symptoms
Biomarkers: CSF mRNAs and proteins

### Targeting Glycogen Synthase for Myoclonic Epilepsy of Lafora

Laforin (EPM2A) gene mutations causes AR Myoclonic Epilepsy.
Disease: AR inherited progressive seizure disorder
Medical Need: Lethal disorder of seizures and cognitive decline with no disease-modifying therapy
Prevalence: 4 per 1,000,000
Target Validation: Excellent via human molecular genetics; mutations causes AR familial Myoclonic Epilepsy of Lafora
Target tissue: CNS
Mechanism: Autosomal recessive dysfunction of Laforin causes misfolding of glycogen and foci for seizures
Efficacy: 70% KD of Glycogen synthase GYS1
Safety: GYS1 deficiency causes skeletal and cardiac muscle glycogen deficiency. Liver glycogen synthase is GYS2. GYS1 mice that survive have muscle defects.
Diagnosis: Family history; genetic testing; early symptoms
Biomarkers: CSF mRNAs and protein

### Ligands

In certain embodiments, the double-stranded iRNA agent of the invention is further modified by covalent attachment of one or more conjugate groups. In general, conjugate groups modify one or more properties of the attached double-stranded iRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the double-stranded iRNA agent further comprises a targeting ligand that targets a receptor which mediates delivery to a specific CNS tissue. These targeting ligands can be conjugated in combination with the lipophilic moiety to enable specific intrathecal, intracisternal magna, and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells), glucose transporter protein, and LDL receptor ligand.

In some embodiments, the double-stranded iRNA agent further comprises a targeting ligand that targets a receptor which mediates delivery to a specific ocular tissue. These targeting ligands can be conjugated in combination with the lipophilic moiety to enable specific intravitreal and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH (SEQ ID NO: 1) or Cyclo(-Arg-Gly-Asp-D-Phe-Cys (SEQ ID NO: 2); LDL receptor ligands; and carbohydrate based ligands (which targets_endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacrylic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., α-helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA; SEQ ID NO: 3); AALAEALAEALAEALAEALAEALAAAAGGC (EALA; SEQ ID NO: 4); ALEALAEALEALAEA (SEQ ID NO: 5); GLFEAIEGFIENGWEGMIWDYG (INF-7; SEQ ID NO: 6); GLFGAIAGFIENGWEGMIDGWYG (Inf HA-2; SEQ ID NO: 7); GLFEAIEGFIENGWEGMIDGWYGCGLFEAIEGFIENGWEGMIDGWYGC (diINF-7; SEQ ID NO: 8); GLFEAIEGFIENGWEGMIDGGCGLFEAIEGFIENGWEGMIDGGC (diINF-3; SEQ ID NO: 9); GLFGALAEALAEALAEHLAEALAEALEALAAGGSC (GLF; SEQ ID NO: 10); GLFEAIEGFIENGWEGLAEALAEALEALAAGGSC (GALA-INF3; SEQ ID NO: 11); GLFEAIEGFIENGWEGnIDGKGLFEAIEGFIENGWEGnIDG (INF-5, n is norleucine; SEQ ID NO: 12); LFEALLELLESLWELLLEA (JTS-1; SEQ ID NO: 13); GLFKALLKLLKSLWKLLLKA (ppTG1; SEQ ID NO: 14); GLFRALLRLLRSLWRLLLRA (ppTG20; SEQ ID NO: 15); WEAKLAKALAKALAKHLAKALAKALKACEA (KALA; SEQ ID NO: 16); GLFFEAIAEFIEGGWEGLIEGC (HA; SEQ ID NO: 17); GIGAVLKVLTTGLPALISWIKRKRQQ (Melittin; SEQ ID NO: 18); H₅WYG (SEQ ID NO: 19); and CHK₆HC (SEQ ID NO: 20).

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin; SEQ ID NO: 21); GRKKRRQRRRPPQC (Tat fragment 48-60; SEQ ID NO: 22); GALFLGWLGAAGSTMGAWSQPKKKRKV (signal sequence based peptide; SEQ ID NO: 23); LLIILRRRIRKQAHAHSK (PVEC; SEQ ID NO: 24); GWTLNSAGYLLKINLKALAALAKKIL (transportan; SEQ ID NO: 25); KLALKLALKALKAALKLA (amphiphilic model peptide; SEQ ID NO: 26); RRRRRRRRR (Arg9; SEQ ID NO: 27); KFFKFFKFFK (Bacterial cell wall permeating peptide; SEQ ID NO: 28); LLGDFFRKSKEKIGKEFKRIVQRIKDFLRNLVPRTES (LL-37; SEQ ID NO: 29); SWLSKTAKKLENSAKKRISEGIAIAIQGGPR (cecropin P1; SEQ ID NO: 30); ACYCRIPACIAGERRYGTCIYQGRLWAFCC (α-defensin; SEQ ID NO: 31); DHYNCVSSGGQCLYSACPIFTKIQGTCYRGKAKCCK (β-defensin; SEQ ID NO: 32); RRRPRPPYLPRPRPPPFFPPRLPPRIPPGFPPRFPPRFPGKR-NH2 (PR-39; SEQ ID NO: 33); ILPWKWPWWPWRR-NH2 (indolicidin; SEQ ID NO: 34); AAVALLPAVLLALLAP (RFGF; SEQ ID NO: 35); AALLPVLLAAP (RFGF analogue; SEQ ID NO: 36); and RKCRIVVIRVCR (bactenecin; SEQ ID NO: 37).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-gulucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the double-stranded iRNA agent of the invention (e.g., double-stranded iRNA agent of the invention or linker). In some embodiments, the ligand can be incorporated via coupling to a “precursor” monomer after said “precursor” monomer has been incorporated into a component of the double-stranded iRNA agent of the invention (e.g., double-stranded iRNA agent of the invention or linker). For example, a monomer having, e.g., an amino-terminated tether (i.e., having no associated ligand), e.g., monomer-linker-NH₂ can be incorporated into a component of the compounds of the invention (e.g., a double-stranded iRNA agent of the invention or linker). In a subsequent operation, i.e., after incorporation of the precursor monomer into a component of the compounds of the invention (e.g., double-stranded iRNA agent of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the double-stranded iRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the double-stranded iRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In certain embodiments, the double-stranded iRNA agent of the invention comprises a ligand of Formula (II), (III), (IV) or (V):

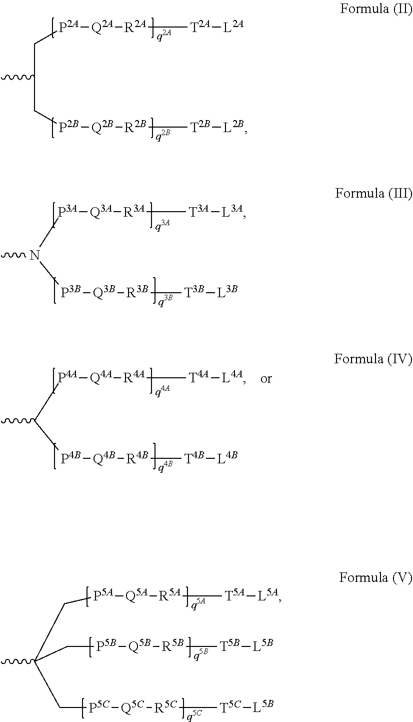

wherein:
q^{2A}, q^{2B}, q^{3A}, q^{3B}, q4^{A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

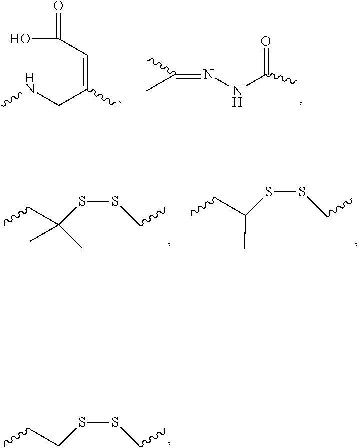

or heterocyclyl;
L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence O or S;
p represent independently for each occurrence 0-20.

As discussed above, because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradant can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an siRNA compound on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA compound and a drug that affects the uptake of the siRNA into the cell. The drug can be administered before, after, or at the same time that the siRNA compound is administered. The drug can be covalently or non-covalently linked to the siRNA compound. The drug can be, for example, a lipopolysaccharide, an activator of p38 MAP kinase, or an activator of NF-κB. The drug can have a transient effect on the cell. The drug can increase the uptake of the siRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilot II reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

Making Double-Stranded iRNA Agents Conjugated to a Lipophilic Moiety

In some embodiments, the lipophilic moiety is conjugated to the double-stranded iRNA agent via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a lipophilic moiety is conjugated to a nucleobase, the preferred position is one that does not interfere with hybridization, i.e., does not interfere with the hydrogen bonding interactions needed for base pairing. In one embodiment, the lipophilic moieties may be conjugated to a nucleobase via a linker containing an alkyl, alkenyl or amide linkage. Exemplary conjugations of the lipophilic moieties to the nucleobase are illustrated in FIG. 1 and Example 7.

Conjugation to sugar moieties of nucleosides can occur at any carbon atom. Exemplary carbon atoms of a sugar moiety that a lipophilic moiety can be attached to include the 2′, 3′, and 5′ carbon atoms. A lipophilic moiety can also be attached to the 1′ position, such as in an abasic residue. In one embodiment, the lipophilic moieties may be conjugated to a sugar moiety, via a 2′-o modification, with or without a linker. Exemplary conjugations of the lipophilic moieties to the sugar moiety (via a 2′-O modification) are illustrated in FIG. 1 and Examples 1, 2, 3, and 6.

Internucleosidic linkages can also bear lipophilic moieties. For phosphorus-containing linkages (e.g., phosphodiester, phosphorothioate, phosphorodithioate, phosphoroamidate, and the like), the lipophilic moiety can be attached directly to the phosphorus atom or to an O, N, or S atom bound to the phosphorus atom. For amine- or amide-containing internucleosidic linkages (e.g., PNA), the lipophilic moiety can be attached to the nitrogen atom of the amine or amide or to an adjacent carbon atom.

There are numerous methods for preparing conjugates of oligonucleotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant lipophilic moiety, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, a lipophilic molecule having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the lipophilic molecule has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the lipophilic molecule having the phosphoramidite group may include phosphitylation of a free hydroxyl to generate the phosphoramidite group.

Synthesis procedures of lipophilic moiety-conjugated phosphoramidites are exemplified in Examples 1, 2, 4, 5, 6, and 7. Examples of procedures of post-synthesis conjugation of lipophilic moieties or other ligands are illustrated in Example 3.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif.), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### Pharmaceutical Compositions

In one aspect, the invention features a pharmaceutical composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the siRNA compound (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one example the pharmaceutical composition includes an siRNA compound mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an siRNA compound, e.g., a double-stranded siRNA compound, or ssiRNA compound (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicylate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorochemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a form suitable for oral delivery. In one embodiment, oral delivery can be used to deliver an siRNA compound composition to a cell or a region of the gastro-intestinal tract, e.g., small intestine, colon (e.g., to treat a colon cancer), and so forth. The oral delivery form can be tablets, capsules or gel capsules. In one embodiment, the siRNA compound of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound and a delivery vehicle. In one embodiment, the siRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a rectal dosage form. In one embodiment, the rectal dosage form is an enema. In another embodiment, the rectal dosage form is a suppository.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a vaginal dosage form. In one embodiment, the vaginal dosage form is a suppository. In another embodiment, the vaginal dosage form is a foam, cream, or gel.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a pulmonary or nasal dosage form. In one embodiment, the siRNA compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell of a subject via administration to a subarachnoid space of the subject, thereby contacting said cell with the double-stranded iRNA agent of the invention.

Another aspect of the invention relates to a method of reducing the expression of a target gene in a subject, comprising administering to a subarachnoid space of the subject the double-stranded iRNA agent of the invention.

Another aspect of the invention relates to a method of treating a subject having a CNS disorder, comprising administering to a subarachnoid space of the subject a therapeutically effective amount of the double-stranded RNAi agent of the invention, thereby treating the subject. Exemplary CNS disorders that can be treated by the method of the invention include Alzheimer, amyotrophic lateral sclerosis (ALS), frontotemporal dementia, Huntington, Parkinson, spinocerebellar, prion, and lafora.

In embodiments, the double-stranded iRNA agent of the invention can be delivered to a subject by intracisternal magna (ICM) administration, including by ICM injection.

In one embodiment, the double-stranded iRNA agent is administered intrathecally or via ICM. By intrathecal administration of the double-stranded iRNA agent, the method can reduce the expression of a target gene in a brain or spine tissue, for instance, cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG). By ICM administration of the double-stranded iRNA agent, the method can reduce the expression of a target gene in a brain or spine tissue, for example, the cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG).

In some embodiments, exemplary target genes are APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25. To reduce the expression of these target genes in the subject, the double-stranded iRNA agent can be administered intravitreally.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. In addition to administration to a subarachnoid space of a subject, it is further contemplated that additional routes of administration could be employed, including, without limitation, topical (including ophthalmic, vaginal, rectal, intranasal, transdermal), oral or parenteral. Parenteral administration includes intravenous drip, subcutaneous, intraperitoneal or intramuscular injection, or intrathecal or intraventricular or ICM administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular or ICM administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the double-stranded iRNA agent is delivered by intrathecal injection (i.e. injection into the spinal fluid which bathes the brain and spinal cord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Intracisternal Administration (ICM). In one embodiment, the double-stranded iRNA agent is delivered by ICM injection (i.e. injection into the CSF in the cisterna magna). ICM administration may involve injection of a therapeutic oligonucleotide through the atlano-occipital membrane and into the cisterna magna space. Advantageously, ICM administration has demonstrated ability to deliver oligonucleotide therapeutics to a variety of CNS tissues including, but not limited to, the cerebral cortex (e.g., frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG).

The ICM injection may include a stereotactic-guided injection system that includes a 3-way stopcock have a male Luer fitting and two female Luer fittings. A 22-gauge 1 inch spinal needle may be attached to the male Luer lock fitting, a sterile syringe may be connected to the female Luer fitting opposite the male Luer lock fitting, and a tube may be connected to the second female Luer fitting. Optionally, the tube connected to the second female Luer fitting may be a loading line, which may in turn be connected to an infusion pump.

Generally, ICM delivery may involve aligning the spinal needle with the cervical gap of an anesthetized subject or patient, and then manually inserting the spinal needle through the skin and then through the atlanto-occipital membrane (positioned between the dorsal surface of the medulla oblongata and the cerebellum) and into the cisterna magna. To avoid damage to the medulla oblongata by over insertion of the spinal needle, it is important to verify the correct needle insertion depth. This may be accomplished by applying negative pressure to the needle (e.g., by partially retracting the syringe), and maintaining this negative pressure throughout the ICM administration. Once the spinal needle has passed through the atlanto-occipital membrane and into the cisterna magna, a small amount of CSF may be withdrawn into the syringe to serve as a baseline control if desired. The 3-way stopcock may then be opened to allow the loading line to deliver an oligonucleotide therapeutic at a desired rate (e.g., 0.5 mL/minute) until the desired amount of therapeutic has been delivered (e.g., 25 mg, 50 mg, etc.). After a resting period (e.g., about 5 minutes), the spinal needle may be slowly retracted out of the cisterna magna. Direct pressure may be used to achieve hemostasis after the spinal needle is completely removed. The subject or patient may then be monitored until full recovery from the anesthesia.

The amount of ICM injected iRNA agents may vary from one target gene to another target gene and the appropriate amount that has to be applied may have to be determined individually for each target gene and subject class (e.g., human subject). Typically, this amount ranges between 10 g to 2 g, such as 1 mg to 1500 mg, in a suitable injection volume (e.g., 1-30 mL).

For ease of exposition, the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. In some embodiments, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) is delivered to a subject via ICM administration.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, trehalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like. Suitable pH adjusters or buffers include organic salts prepared from organic acids and bases, such as sodium citrate, sodium ascorbate, and the like; sodium citrate may be used in some embodiments.

### Kits

In certain other aspects, the invention provides kits that include a suitable container containing a pharmaceutical formulation of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). In certain embodiments the individual components of the pharmaceutical formulation may be provided in one container. Alternatively, it may be desirable to provide the components of the pharmaceutical formulation separately in two or more containers, e.g., one container for an siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention, and are not intended to limit the invention.

### Example 1: AD-454844

AD-454844 is an APP-targeting duplex that has been described previously. For example, see the synthetic methods described in International patent application PCT/US2020/037928, published as WO2020/257194 on Dec. 24, 2020, which is hereby incorporated by reference in its entirety.

AD-454844 modified sequences are shown in Table 2, while unmodified sequences are shown in Table 3.

TABLE 2
APP-Targeting Duplexes, Modified Sequences

| | Sense | | SEQ | | | SEQ | | SEQ |
|---|---|---|---|---|---|---|---|---|
| Duplex | Oligo | | ID | Antisense | | ID | | ID |
| Name | Name | Oligo Seq | NO: | Oligo Name | Oligo Seq | NO: | mRNA target sequence | NO: |
| AD- | A- | asasaau(Chd)CfaAfCfCfuaca | 38 | A-882382.1 | VPusGfsaacu(Tgn)guagguUfgGfauuuuscsg | 39 | CGAAAAUCCAACCUACAAGUUCU | 40 |
| 454844 | 882381.1 | aguuscsa | | | | | | |
| AD- | A- | asasaau(Chd)CfaAfCfCfuaca | 38 | A-2394037.1 | VPusGfsaacu(Tgn)guagguUfgGfau(Uah)uuscsg | 41 | CGAAAAUCCAACCUACAAGUUCU | 40 |
| 1395836 | 882381.1 | aguuscsa | | | | | | |
| AD- | A- | asasaau(Chd)CfaAfCfCfuaca | 38 | A-2497737.1 | VPusGfsaacu(Tgn)guagguUfgGfauY196uuscsg | 42 | CGAAAAUCCAACCUACAAGUUCU | 40 |
| 1397045 | 882381.1 | aguuscsa | | | | | | |

Table 2 key: VP=5′-Vinyl phosphonate, a=2′-O-methyladenosine-3′-phosphate, c=2′-O-methylcytidine-3′-phosphate. g=2′-O-methylguanosine-3′-phosphate, u=2′-O-methyluridine-3′-phosphate, Af=2′-deoxy-2′-fluoroadenosine-3′-phosphate, Cf=2′-deoxy-2′-fluorocytidine-3′-phosphate, Gf=2′-deoxy-2′-fluoroguanosine-3′-phosphate, Uf=2′-deoxy-2′-fluorouridine-3′-phosphate, Afs=2′-deoxy-2′-fluoroadenosine-3′-phosphorothioate, Cfs=2′-deoxy-2′-fluorocytidine-3′-phosphorothioate, Gfs=2′-deoxy-2′-fluoroguanosine-3′-phosphorothioate, Ufs=2′-deoxy-2′-fluorouridine-3′-phosphorothioate, as=2′-O-methyladenosine-3′-phosphorothioate, cs=2′-O-methylcytidine-3′-phosphorothioate, gs=2′-O-methylguanosine-3′-phosphorothioate, us=2′-O-methyluridine-3′-phosphorothioate, (Tgn)=Thymidine-glycol nucleic acid (GNA) S-Isomer, (Chd)=2′-O-hexadecyl-cytidine-3′-phosphate, (Uah)=2′-O-(6-aminohexyl)-uridine-3′-phosphate, Y196=2′-O-(6-aminohexyl-metaiodobenzoyl)-uridine-3′-phosphate ((Uah) with iodo-benzoyl).

TABLE 3
APP-Targeting Duplexes, Unmodified Sequences

| | | | SEQ | | | | SEQ | |
|---|---|---|---|---|---|---|---|---|
| Duplex | Sense Oligo | Sense | ID | Source | Antisense | Antisense | ID | Source |
| Name | Name | Sequence (5′ to 3′) | NO: | (Range) | Oligo Name | Sequence (5′ to 3′) | NO: | (Range) |
| AD-454844 | A- | AAAAUCCAACCUACAAGUUCA | 43 | NM_000484.4 | A-882382.1 | UGAACUTGUAGGUUGGAUUUUCG | 44 | NM_000484.4 |
| | 882381.1 | | | (2423-2443) | | | | (2421-2443) |
| AD-1395836 | A- | AAAAUCCAACCUACAAGUUCA | 43 | NM_000484.4 | A-2394037.1 | UGAACUTGUAGGUUGGAUUUUCG | 44 | NM_000484.4 |
| | 882381.1 | | | (2423-2443) | | | | (2421-2443) |
| AD-1397045 | A- | AAAAUCCAACCUACAAGUUCA | 43 | NM_000484.4 | A-2497737.1 | UGAACUTGUAGGUUGGAUUUUCG | 44 | NM_000484.4 |
| | 882381.1 | | | (2423-2443) | | | | (2421-2443) |

### Soluble APP Alpha Soluble APP Beta

CSF levels of sAPPα and sAPPβ were determined utilizing a sandwich immunoassay MSD® 96-well MULTI-SPOT sAPPα/sAPPβ assay (Catalog no. K15120E; Meso Scale Discovery, Rockville, MD, USA) according to the manufacturer's protocol with some modifications. The standards, blanks, and non-human primate CSF samples (8× dilution) were prepared with the 1% Blocker-A/TBST (provided in the kit). Pre-coated plate (provided in the kit) was blocked with 150 μL/well of 3% Blocker A/TBST solution at room temperature for 1 hour with shaking. After three washes with 1×TBST, 25 μL/well of prepared standard, blanks, and CSF samples were added to the plate in two replicates and incubated for 1 hour at room temperature with shaking. Following subsequent plate washes, 50 μL/well of detection antibody prepared in 1% Blocker A/TBST (50× dilution) was added and incubated at room temperature for 1 hour with shaking. After plate washes, 1× Read Buffer T was added to the plate and incubated for 10 minutes at room temperature (without shaking) before imaging and analyzing in MSD QuickPlex Imager.

Raw data were analyzed using SoftMax Pro, version 7.1 (Molecular Devices). A 5-parameter, logistic curve fitting with 1/Y² weighing function was used to model the individual calibration curves and calculate the concentration of analytes in the samples.

### Beta-Amyloid Panel (Aβ40, Aβ38, Aβ42)

CSF levels of Beta-amyloid (Aβ40, Aβ38, Aβ42) were determined utilizing a sandwich immunoassay multiplex kit MSD® 96-well MULTI-SPOT AB Peptide Panel 1 V-Plex (Catalog No. K15200E, Meso Scale Discovery, Rockville, MD, USA) according to the manufacturer's protocol with some modifications. The standards, blanks, and non-human primate CSF (8× dilution) were prepared with Diluent 35 (provided in the kit). Detection antibody (supplied at 50×) was prepared at a working concentration of 1× in Diluent 100 (provided in the kit) combined with 30 μL of Aβ40 Blocker. Pre-coated plate (provided in the kit) was blocked with 150 μL/well with Diluent 35 for 1 hour at room temperature with shaking. After three washes with 1×PBST, 25 μl/well of prepared detection antibody solution was added to the plate. Following with the addition of 25 μL/well of prepared standards, blanks, and samples in two replicates, plate was incubated at room temperature for 2 hours with shaking. Following subsequent plate washes, 150 μL/well of 2× Read buffer T was added and plate was imaged and analyzed in the MSD QuickPlex Imager immediately.

Raw data were analyzed using SoftMax Pro, version 7.1 (Molecular Devices, San Jose, CA, USA). A 4-parameter, logistic curve fitting with 1/Y² weighing function was used to model the individual calibration curves and calculate the concentration of analytes in the samples.

### Mass Spec Method

Drug concentrations in plasma, CSF and CNS tissue samples were quantitated using a qualified LC-MS/MS method. Briefly, tissue samples were homogenized in lysis buffer, then the oligonucleotides were extracted from plasma, CSF or tissue lysate by solid phase extraction and analyzed using ion-pairing reverse phase liquid chromatography coupled with mass spectrometry under negative ionization mode. The concentration of the full-length antisense strand of the dosed duplex was measured. The drug concentrations were reported as the antisense-based duplex concentrations. The calibration range is 10-5000 ng/mL for plasma and CSF samples, and 100-50000 ng/g for CNS tissue samples. Concentrations that were calculated below the LLOQ are reported as <LLOQ. An analog duplex with different molecular weight was used as internal standard.

mRNA Knockdown by qPCR Method

Total RNA was isolated from rat brain and spinal cord tissue samples using the miRNeasy Mini Kit from (Qiagen, Catalog No. 217004) according to the manufacturer's instructions. Following isolation, RNA was reverse transcribed using SuperScript™ IV VILO™ Reverse Transcriptase (Thermo Fisher Scientific). Quantitative PCR analysis was performed using a ViiA7 Real-Time PCR System from Thermo Fisher Scientific of Waltham MA 02451 (Catalog No. 4453537) with Taqman Fast Universal PCR Master Mix (Applied Biosystems Catalog No. 4352042), pre-validated amyloid beta precursor protein (APP) (Mf01552291_m1) and peptidylprolyl isomerase B (PPIB) (Mf02802985_m1) Taqman Gene Expression Assays (Thermo Fisher Scientific).

The relative reduction of APP mRNA was calculated using the comparative cycle threshold (Ct) method. During qPCR, the instrument sets a baseline in the exponential phase of the amplification curve and assigns a Ct value based on the intersection point of the baseline with the amplification curve. The APP mRNA reduction was normalized to the experimental untreated control group as a percentage for each respective group using the Ct values according to the following calculations: ΔCt_{App}=Ct_{App}−Ct_{Ppib}

ΔΔCt_{App}=ΔCt_{App}−ΔCt_{untreated control group mean}

Relative mRNA level=2^{-ΔΔCt}

### Example 2: Cerebrospinal Fluid Biomarkers Correlate with mRNA KD and Drug Levels in the Central Nervous System Following Intracisternal Magna Injection in Nonhuman Primates

To test the ability of intracisternal magna (ICM) injection to effectively deliver siRNA to various regions of the central nervous system (CNS), AD-454844 targeting APP was given to cynomolgus monkeys via ICM administration and both drug levels and mRNA knockdown were assessed at day 15 post dose.

As shown in Table 4, drug levels were clinically significant in multiple CNS tissues at day 15 post ICM dose, including the prefrontal cortex, striatum, hypothalamus, cervical spine and lumbar spine. For example, in prefrontal cortex tissue the measured drug levels ranged from 1,279 ng/g to 40,879 ng/g. In the striatum, the measured drug levels ranged from 191 ng/g to 2683 ng/g. In the hypothalamus, the measured drug levels ranged from 1,293 ng/g to 27,803 ng/g. Significant levels were also observed in the spine. For example, measured drug levels in the cervical spine ranged from 6,485 ng/g to 33,089 ng/g, while drug levels in the lumbar spine ranged from 2,924 ng/g to 68,621 ng/g.

The relatively low drug levels for P0101 and P0104 are believed to be due to a dosing artifact arising from variability of lumbar puncture dosing. One explanation for this is hypothesized that the dose did not make it into the intrathecal space; rather it was administered epidurally (outside the meninges instead of inside).

TABLE 4
Intracisternal Magna-Tissue siRNA Levels

| Animal | | Study dose | | | Duplex Final Conc. | |
|---|---|---|---|---|---|---|
| ID | Day | (mg) | Matrix | Assay | (ng/g) | Note |
| P0101 | D15 | 45 | BRAIN_PFRONTAL | LC- | 1741 | |
| | | | | MS/MS | | |
| P0102 | D15 | 45 | | LC- | 40879 | |
| | | | | MS/MS | | |
| P0103 | D15 | 45 | | LC- | 31432 | |
| | | | | MS/MS | | |
| P0104 | D15 | 45 | | LC- | 1279 | |
| | | | | MS/MS | | |
| P0101 | D15 | 45 | BRAIN_STRIATUM | LC- | 191 | |
| | | | | MS/MS | | |
| P0102 | D15 | 45 | | LC- | 2683 | |
| | | | | MS/MS | | |
| P0103 | D15 | 45 | | LC- | 2453 | |
| | | | | MS/MS | | |
| P0104 | D15 | 45 | | LC- | <LLOQ | |
| | | | | MS/MS | | |
| P0101 | D15 | 45 | BRAIN_ | LC- | 1293 | |
| | | | HYPOTHALAMUS | MS/MS | | |
| P0102 | D15 | 45 | | LC- | 27803 | |
| | | | | MS/MS | | |
| P0103 | D15 | 45 | | LC- | 19220 | |
| | | | | MS/MS | | |
| P0104 | D15 | 45 | | LC- | 531 | |
| | | | | MS/MS | | |
| P0101 | D15 | 45 | SPINE_CERVICAL | LC- | 6625 | |
| | | | | MS/MS | | |
| P0102 | D15 | 45 | | LC- | 33089 | |
| | | | | MS/MS | | |
| P0103 | D15 | 45 | | LC- | 22298 | |
| | | | | MS/MS | | |
| P0104 | D15 | 45 | | LC- | 6485 | |
| | | | | MS/MS | | |
| P0101 | D15 | 45 | SPINE_LUMBAR | LC- | 10275 | |
| | | | | MS/MS | | |
| P0102 | D15 | 45 | | LC- | 68621 | a |
| | | | | MS/MS | | |
| P0103 | D15 | 45 | | LC- | 20526 | |
| | | | | MS/MS | | |
| P0104 | D15 | 45 | | LC- | 2924 | |
| | | | | MS/MS | | |

| | LLOQ | ULOQ |
|---|---|---|
| Analyte | (ng/g) | (ng/g) |
| AD-454844 | 100 | 50000 |

a: Measured concentration was slightly above ULOQ and was accepted.

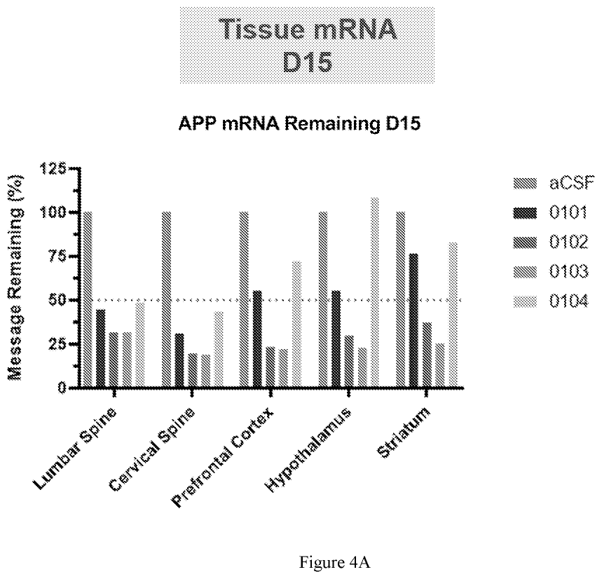

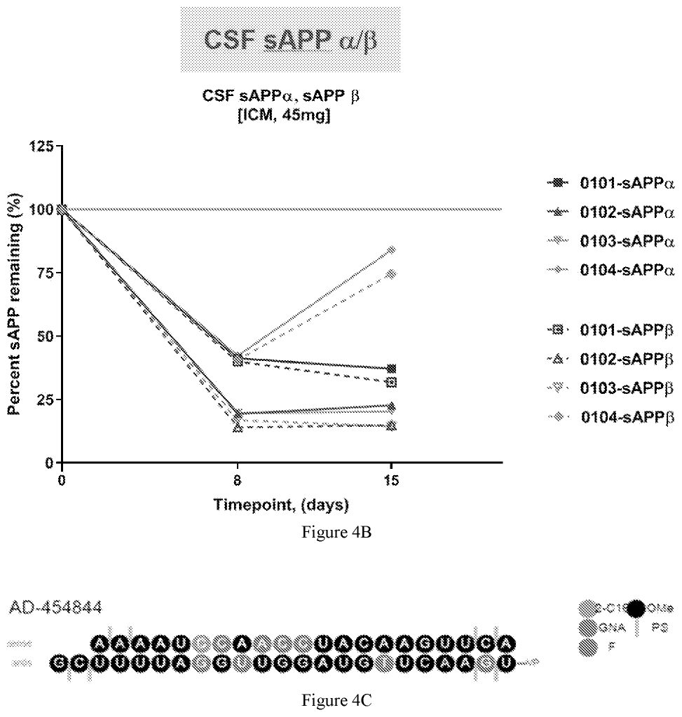

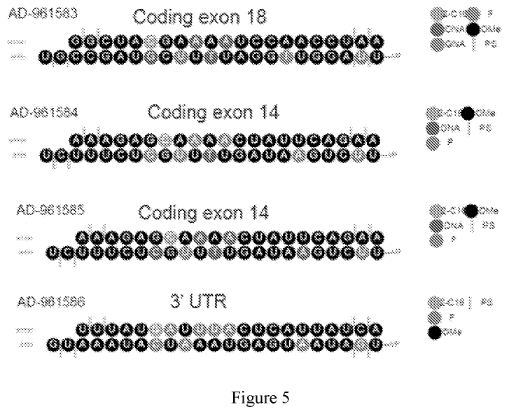

FIG. 4A shows that APP mRNA knockdown with AD-454844, by animal number, in the lumbar spine, cervical spine, prefrontal cortex, hypothalamus, and striatum correlates well with the measured drug levels shown in Table 4. Moreover, greater sAPP lowering was observed in animals with higher tissue drug levels.

Additionally, CSF was collected from the cisterna magna and soluble APP alpha and beta were measured at Day 8, Day 15 post dose of AD-454844. As shown in FIG. 4B, ICM dosing of AD-454844 results in sufficient siRNA delivery throughout the spine and brain to produce APP mRNA knockdown at the tissue level. Surprisingly, knockdown in the tissue corresponds to silencing of target engagement biomarkers in the CSF as early as one week post dose. The structure of AD-454844 is shown in FIG. 4C.

### Example 3: ICM Protocol

Animals. As shown in Table 5, ten female cynomolgus monkeys were transferred to the study site and were acclimated to the study room for 15 days prior to the initiation of dosing. At initiation of dosing, animals were 37 to 59 months old, and body weights ranged from 2.5 to 4.5 kg. Water was provided ad libitum, and animals were offered Certified Primate Diet #5048 (PMI Nutrition International Certified LabDiet®) one or two times daily, unless fasted for study procedures.

Female cynomolgus monkeys were assigned to three groups, and doses were administered as indicated in the following table. Animals in Groups 1 and 2 were dosed via intracisternal injection. Animals in Groups 3 were dosed via intrathecal injection. All doses were administered as a single dose on Day 1 of the dosing phase at a target volume of 2 mL. The vehicle control article was artificial cerebral spinal fluid (aCSF), provided by the Sponsor as a pre formulated solution.

TABLE 5
Study Design

| | No. of | | Dose |
|---|---|---|---|
| | Animals | Dose Level | Concentration |
| Group^{a} | Female | (mg/animal) | (mg/mL) |
| 1 (Intracisternal [ICM]) | 1 | 0 | 0 |
| 2 (Intracisternal [ICM]) | 4 | 45 | 22.5 |
| 3 (Intrathecal [IT]) | 5 | 120 | 60 |

^{a}Group 1 was dosed with vehicle control article only.

| Test Article^{a} | Storage | Lot Number | Assay^{b} |
|---|---|---|---|
| AD-454844 | In a refrigerator | AD-454844.28 | 60 mg/mL |
| | (2 to 8° C.) | | |

^{a}Test article was formulated vials at a concentration of 60 mg/mL.
^{b}Assay was based on single-strand concentration.

| Vehicle Control Article^{a} | Storage |
|---|---|
| Artificial cerebral spinal fluid (aCSF) | In a refrigerator |
| | (2 to 8° C.) |

^{a}Provided by the Sponsor as pre-formulated solution

All dose formulations were prepared on the day of dosing under aseptic conditions according to the mixing procedures and apportioned for use. For Group 1, the vehicle control article was used as supplied. For Group 2, the test article (60 mg/mL) was diluted volume:volume with vehicle control article to achieve the required dose concentration. For Group 3, the test article (60 mg/mL) was used as supplied. The vehicle control article (aCSF) was also dispensed for use as a flush. Dose formulations and flush were stored in a refrigerator, set to maintain 2 to 8° C. Assessment of toxicity was based on mortality, clinical observations, body weights, qualitative food consumption, neurological observations, physical examinations, and clinical pathology. Blood and cerebrospinal fluid (CSF) samples were collected for potential toxicokinetic evaluations.

Dose Administration. Dose formulations were allowed to equilibrate to approximately room temperature for at least 30 minutes prior to dosing. Dose formulations were administered once on Day 1 of the dosing phase at a dose volume of 2 mL. Animals were fasted for at least 12 hours prior to dosing. For each Group, prior to the first dose and as needed thereafter, the skin over the dosing region was clipped free of hair to allow clear observation of the dose site. Animals were anesthetized with ketamine and dexmedetomidine prior to dosing. Following administration, each animal was administered a reversal agent and allowed to recover. Buprenorphine was used as needed. Unless otherwise stated, postdose events were based on the completion of dosing (including the flush) for each animal.

Animal in Groups 1 and 2 were dosed by ICM injection into the cisterna magna. The ICM injection was administered via slow bolus push of at least 3 minutes in the cisterna magna. Up to 1 mL of CSF was withdrawn prior to dosing using an Echogenic Needle 22 G×2″. Following dosing, the needle was flushed with approximately 0.25 mL of aCSF to clear the needle.

Animals in Group 3 were dosed by IT injection via slow bolus push of at least 3 minutes in the lumbar region at three dose sires: site A was between L5 and L6, site B was between L4 and L5, and site C was between L3 and L4. Up to 1 mL of CSF was withdrawn prior to dose administration using Pencan Pencil Point Spinal Needle 25 G×1″. Following dosing, the needle was flushed with approximately 0.25 mL of aCSF to clear the needle.

Sample Collection. Blood samples (approximately 1.0 mL) were collected via a femoral vein on Days 1, 8, and 15 of the dosing phase. Samples from Day 1 of the dosing phase were collected predose and approximately 30 minutes and 4 and 24 hours postdose. Samples on Days 8 and 15 of the dosing phase were collected once. Animals were not fasted for sample collections, unless fasted for other study procedures. Blood was collected into tubes containing potassium (K₂) EDTA as the anticoagulant. Samples were maintained on chilled cryoracks prior to and after centrifugation and were centrifuged within 1 hour of collection. Plasma was harvested and stored on dry ice or in a freezer set to maintain −60 to −80° C. until analyzed.

Cerebrospinal fluid samples were collected on Days 1, 8, and 15 of the dosing phase from the cisterna magna (excluding the Day 1 predose samples from Group 3, which were collected via lumbar puncture). On Day 1 of the dosing phase, CSF samples were collected predose and approximately 4 and 24 hours postdose. On Days 8 and 15 of the dosing phase, CSF samples were collected once. Target CSF sample volumes were up to 1 mL for the Day 1 predose collection, approximately 0.4 mL for the remaining nonterminal collections, and as much as possible (up to 3 mL) for terminal collections. For each CSF sample collected, a visual inspection was performed to examine if the sample appeared to be contaminated with blood (e.g., pinkish or cloudy appearance), and the observation was recorded. Up to three attempts were made and the least-contaminated sample was prepared for analysis (if applicable). The other samples were discarded. Samples were held on chilled cryoracks prior to and after centrifugation and were centrifuged within 1 hour of collection. Following centrifugation, supernatant was harvested and divided into two approximately equal aliquots. Samples were stored on dry ice or in a freezer, set to maintain −60 to −80° C. until analyzed.

Clinical Observations. Animals were checked twice daily (a.m. and p.m.) for mortality, abnormalities, and signs of pain or distress. Cageside observations were conducted for each animal once daily during the predose, and dosing phases, except on days when detailed observations were conducted. Detailed observations were conducted for each animal three times during the predose phase, on Days 1, 8, and 14 of the dosing phase, and on the day of scheduled sacrifice.

Body weights were recorded three times during the predose phase and on Days 1, 8, and 14 of the dosing phase.

Qualitative food consumption was recorded once daily during the predose and dosing phases, except on the day of animal transfer or unless fasted for other study procedures.

Physical examinations were conducted once during the predose phase, prior to dosing on Day 1 of the dosing phase, and approximately 4 and 24 hours postdose. Animals were not anesthetized prior to examination.

Neurological examinations were conducted once during the predose phase, prior to dosing on Day 1 of the dosing phase, approximately 4 and 24 hours postdose, and on the day of scheduled sacrifice.

No AD-454844-related mortality occurred. No AD-454844-related effects on body weight, body weight gain, qualitative food consumption, physical examinations, neurological examinations, or clinical pathology parameters were noted. No AD-454844-related macroscopic observations were noted.

AD-454844-related clinical observations for two out of four animals administered 45 mg/animal intracisternally (ICM) and three out of five animals administered 120 mg/animal intrathecally (IT) included tremors. Animals administered 45 mg/animal ICM were noted with involuntary tremors on 1 or 2 days, with the first observation noted on Day 4 of the dosing phase. Animals administered 120 mg/animal IT were noted with involuntary tremors on 3, 6, or 9 separate days, with the first observation noted on Day 3 of the dosing phase. These observations of involuntary tremors were considered not adverse, as they did not affect the health or wellbeing of the affected animals.

In conclusion, female cynomolgus monkeys when administered vehicle control article (aCSF) or 45 mg/animal AD-454844 via a single ICM injection or 120 mg/animal via a single IT injection resulted in clinical observations of tremors for animals administered 45 mg/animal ICM or 120 mg/animal IT; however, these observations were considered tolerated due to the lack of any adverse impact on the health and wellbeing of the affected animals. Therefore, a single administration of 45 mg/animal ICM or 120 mg/animal IT was tolerated after a 14-day observation period. Additionally, blood, CSF, and tissues samples were collected for various analyses, however, analysis and reporting of those samples, if any, would be conducted outside the scope of this study.

No AD-454844-related alterations in body weight or body weight gain were noted for animals administered 45 mg/animal ICM or 120 mg/animal IT. Similarly, no AD-454844-related observations were noted during physical examinations for animals administered 45 mg/animal ICM or 120 mg/animal IT. Also, no remarkable neurological observations were noted.

Clinical Laboratory Procedures. Blood samples for hematology, coagulation, and clinical chemistry were collected from fasted animals via a femoral vein twice during the predose phase and once on Day 15 of the dosing phase. The anticoagulants were sodium citrate for coagulation tests and potassium EDTA for hematology tests. Samples for clinical chemistry were collected without anticoagulant. The following hematology, coagulation, and clinical chemistry tests were performed:

Hematology Tests

red blood cell (erythrocyte) count white blood cell (leukocyte) count

hemoglobin absolute neutrophil count

hematocrit absolute lymphocyte count

mean corpuscular volume absolute monocyte count

mean corpuscular hemoglobin absolute eosinophil count

mean corpuscular hemoglobin absolute basophil count

concentration absolute large unstained cell count

red cell distribution width blood smear

absolute reticulocyte count

platelet count

Coagulation Tests

prothrombin time activated partial thromboplastin time

fibrinogen

Clinical Chemistry Tests

glucose aspartate aminotransferase

urea nitrogen alanine aminotransferase

creatinine alkaline phosphatase

total protein gamma glutamyltransferase

albumin creatine kinase

globulin calcium

albumin:globulin ratio inorganic phosphorus

total cholesterol sodium

triglycerides potassium

total bilirubin chloride

Hematology and Coagulation Results: No AD-454844-related effects in hematology or coagulation test results were identified in animals administered 45 mg/animal ICM or 120 mg/animals IT.

All differences in hematology and coagulation test results between control and AD-454844-treated animals or between predose and study phase test results for individual animals were consistent with normal variation and considered incidental. Most or all of the following characterized these differences: small magnitude, lack of dose relationship, inconsistency over time, the absence of correlative findings, and/or similarity to differences present before the initiation of dosing.

Clinical Chemistry: No AD-454844-related effects in clinical chemistry test results were identified in animals administered 45 mg/animal ICM or 120 mg/animal IT.

All differences in clinical chemistry test results between control and AD-454844-treated animals or between predose and study phase test results for individual animals were consistent with normal variation and considered incidental. Most or all of the following characterized these differences: small magnitude, lack of dose relationship, inconsistency over time, the absence of correlative findings, and/or similarity to differences present before the initiation of dosing.

Terminal Procedures. On Day 15 of the dosing phase, all surviving animals, having been fasted overnight, were anesthetized with sodium pentobarbital and exsanguinated via whole body perfusion with heparinized sodium nitrite saline. Terminal body weights were recorded for all animals. A macroscopic examination of the external features of the carcass; external body orifices; abdominal, thoracic, and cranial cavities; organs; and tissues was performed. Bone marrow smears (two slides) were prepared from the sternum of each animal at scheduled sacrifices. As shown in Table 6, the following tissues (when present) from each animal were preserved in 10% neutral-buffered formalin, unless otherwise indicated.

TABLE 6
Tissue Samples

| Organ/Tissue | Organ/Tissue |
|---|---|
| animal identification | lesions^{b} |
| brain (left hemisphere)^{a} | liver^{a} |
| ganglion, dorsal root, cervical | lymph node, deep cervical (left) |
| (2, left and right)^{a} | lymph node, inguinal (left) |
| ganglion, dorsal root, thoracic (2, | muscle, biceps femoris |
| left and right)^{a} | spinal cord (cervical, thoracic, and |
| ganglion, dorsal root, lumbar | lumbar)^{a,c} |
| (2, left and right)^{a} | trigeminal ganglion/nerve (left) |
| heart^{a} | |
| injection site(s) | |
| kidney (2)^{a} | |

^{a}Collected in 10% neutral buffered formalin for no more than three overnights and then processed to paraffin block. Following one overnight in 10% NBF, the formalin was decanted and replaced with fresh 10% NBF.
^{b}Only lesions noted on tissues listed in the tissue preservation were collected and processed to block.
^{c}Transverse and vertical oblique sections were trimmed.

Histology. As indicated in the previous table (Necropsy, Organ Weights, and Macroscopic Observations section), tissues from each animal were embedded in paraffin block.

Frozen Tissue Collection for Potential mRNA, Protein, and Bioanalytical Analyses. At the scheduled sacrifice, tissues in the following list were collected from all animals and frozen for potential mRNA, protein, and bioanalytical analyses. Four samples from each tissue/site were collected for animals in Group 1. One to two samples for each tissue/site were collected from animals in Groups 2 and 3.

brain, prefrontal cortex, right

brain, temporal cortex, right

brain, hippocampus, right

brain, caudate nucleus, right

brain, pons/medulla, right

brain, cerebellum, right

brain, hypothalamus

spinal cord, cervical

spinal cord, thoracic

spinal cord, lumbar

ganglion, dorsal root, cervical

ganglion, dorsal root, thoracic

ganglion, dorsal root, lumbar

trigeminal ganglion/nerve (right)

lymph node, deep cervical (right)

lymph node, inguinal

liver, left lateral lobe

kidney, right cortex

heart, apex

muscle, biceps femoris

Brain Procedures. Samples of 0.5 to 1.0 g of the prefrontal cortex, temporal cortex, and cerebellum were collected into separate 15-mL grinding tubes (Catalog #Thermo 2116-0015, one sample/tube), snap-frozen in liquid nitrogen, and collected on dry ice until transferred to a freezer set to maintain −60 to −80° C. Samples of the hippocampus, caudate nucleus, pons/medulla, and hypothalamus were weighed (samples were no larger than 200 mg) and collected into separate 5-mL grinding tubes (Catalog #SPEX 2240-PEF with pre-loaded grinding ball; one sample/tube), snap-frozen in liquid nitrogen, and collected on dry ice until transferred to a freezer set to maintain −60 to −80° C.

Spinal Cord Procedures. Samples from the caudal end of each section of the spinal cord, 0.5 to 1 g each (dura removed) were collected into separate 15-mL grinding tubes (Catalog #Thermo 2116-0015, one sample/tube), snap-frozen in liquid nitrogen, and collected on dry ice until transferred to a freezer set to maintain −60 to −80° C.

Dorsal Root Ganglion Procedures. Representative pair(s) of the dorsal root ganglions (DRGs) were collected from each of the cervical, thoracic, and lumbar regions and placed into an appropriately sized tube, snap-frozen in liquid nitrogen and stored at −60 to −80° C.

Trigeminal Ganglion/Nerve Procedures. Samples were placed into an appropriately sized tube, snap-frozen in liquid nitrogen, and stored at 60 to 80° C.

Lymph Node Procedures. One to four nodes (when possible) were collected into 15-mL grinding tubes (Catalog #Thermo 2116-0015), snap-frozen in liquid nitrogen, and collected on dry ice until transferred to a freezer set to maintain −60 to −80° C.

Systemic Tissue Procedures. Samples of 0.5 to 1.0 g were weighed, collected in a 15-mL grinding tube (Catalog #Thermo 2116-0015), snap-frozen in liquid nitrogen, and collected on dry ice until transferred to a freezer set to maintain −60 to −80° C.

### Example 4. Additional APP-Targeted siRNAs

Examples of further APP-targeted siRNA that may be administered via ICM injection include, for example, AD-961583, AD-961584, AD-961585, and AD-961586, which are shown in Table 7 (sense sequences) and Table 8 (antisense sequences).

TABLE 7
Additional Human APP Modified Sense Sequences and Targets.

| | | | SEQ | | SEQ |
|---|---|---|---|---|---|
| Duplex | | | ID | | ID |
| Name | target | Sense Sequence (5′ to 3') | NO | mRNA Target Sequence | NO |
| AD-961583 | APP | gsgscua(Chd)gadAadAuccaaccusasa | 45 | GGCUACGAAAAUCCAACCUAA | 49 |
| AD-961584 | APP | asasagag(Chd)aAfaAfcuauucagsasa | 46 | AAAGAGCAAAACUAUUCAGAA | 50 |
| AD-961585 | APP | asasagag(Chd)adAadAcuauucagsasa | 47 | AAAGAGCAAAACUAUUCAGAA | 51 |
| AD-961586 | APP | ususuau(Ghd)AfuUfUfAfcucauuauscsa | 48 | UUUAUGAUUUACUCAUUAUCA | 52 |

Table 7 key: u=2′-O-methyluridine-3′-phosphate, us=2′-O-methyluridine-3′-phosphorothioate, a=2′-O-methyladenosine-3′-phosphate, as =2′-O-methyladenosine-3′-phosphorothioate, c=2′-O-methylcytidine-3′-phosphate, cs=2′-O-methylcytidine-3′-phosphorothioate, g=2′-O-methylguanosine-3′-phosphate, gs=2′-O-methylguanosine-3′-phosphorothioate, Gf=2′-deoxy-2′-fluoroguanosine-3′-phosphate, Uf=2′-deoxy-2′-fluorouridine-3′-phosphate, Cf=2′-deoxy-2′-fluorocytidine-3′-phosphate, Af=2′-deoxy-2′-fluoroadenosine-3′-phosphate, (Ahd)=2′-O-hexadecyl-adenosine-3′-phosphate, (Chd)=2′-O-hexadecyl-cytidine-3′-phosphate, (Ghd)=2′-O-hexadecyl-guanosine-3′-phosphate, (Uhd)=2′-O-hexadecyl uridine-3′-phosphate, and VP=5′-Vinyl phosphonate.

TABLE 8
Additional Human APP Modified Antisense Sequences and Targets

| | | | SEQ | | SEQ |
|---|---|---|---|---|---|
| Duplex | | | ID | | ID |
| Name | target | Antisense Sequence (5′ to 3′) | NO | mRNA Target Sequence | NO |
| AD-961583 | APP | VPusUfsaggu(Tgn)ggaudTuUfcdGuagccsgsu | 53 | UUAGGUTGGAUTUUCGUAGCCGU | 57 |
| AD-961584 | APP | VPuUfcugAfauagudTuUfgCfucuuuscsu | 54 | UUCUGAAUAGUTUUGCUCUUUCU | 58 |
| AD-961585 | APP | VPuUfcugdAauagudTuUfgdCucuuuscsu | 55 | UUCUGAAUAGUTUUGCUCUUUCU | 59 |
| AD-961586 | APP | VPusGfsauaAfugaguaaAfuCfauaaasusg | 56 | UGAUAAUGAGUAAAUCAUAAAUG | 60 |

Table 8 key: u=2′-O-methyluridine-3′-phosphate, us=2′-O-methyluridine-3′-phosphorothioate, a=2′-O-methyladenosine-3′-phosphate, as=2′-O-methyladenosine-3′-phosphorothioate, c=2′-O-methylcytidine-3′-phosphate, cs=2′-O-methylcytidine-3′-phosphorothioate, g=2′-O-methylguanosine-3′-phosphate, gs=2′-O-methylguanosine-3′-phosphorothioate, Gf=2′-deoxy-2′-fluoroguanosine-3′-phosphate, Uf=2′-deoxy-2′-fluorouridine-3′-phosphate, Cf=2′-deoxy-2′-fluorocytidine-3′-phosphate, Af=2′-deoxy-2′-fluoroadenosine-3′-phosphate, (Ahd)=2′-O-hexadecyl-adenosine-3′-phosphate, (Chd)=2′-O-hexadecyl-cytidine-3′-phosphate, (Ghd)=2′-O-hexadecyl-guanosine-3′-phosphate, (Uhd)=2′-O-hexadecyl uridine-3′-phosphate, and VP=5′-Vinyl phosphonate.

### Example 5. PET Studies Delineate the Distribution of CNS siRNAs in Non-Human Primates

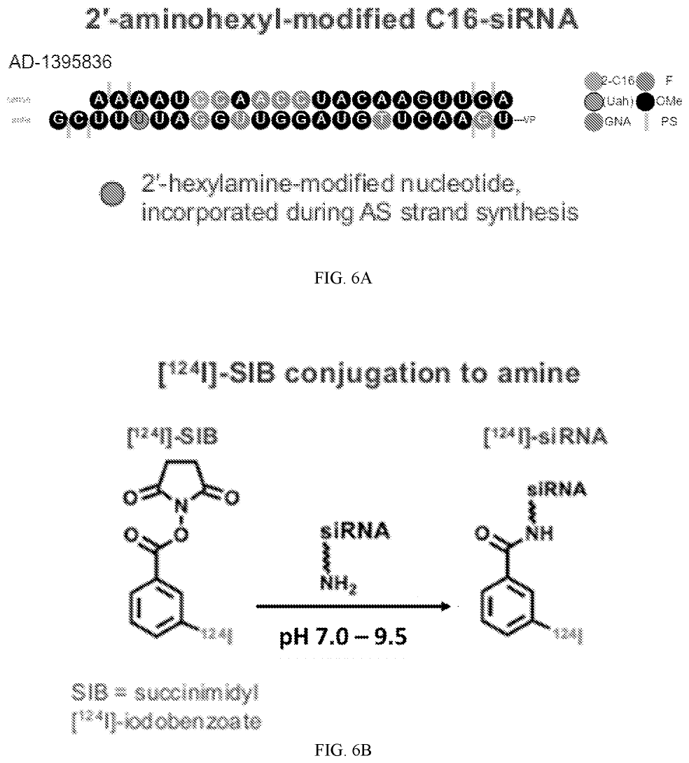

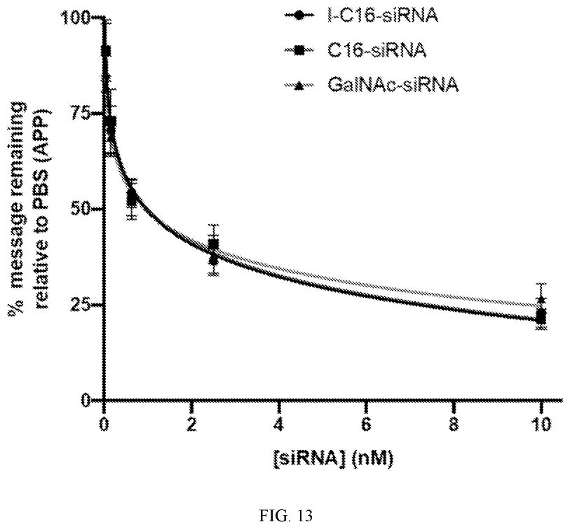

Positron emission tomography (PET) was used to assess the distribution of CNS siRNAs in non-human primates (NHPs). Iodinated C16-siRNAs were generated for use as PET probes. Briefly, a covalently linked radiolabel (e.g., Iodine-124 (¹²⁴I)) was introduced at an internal, metabolically stable position as shown in FIG. 6A and FIG. 6B. ¹²⁴I is a high energy, high specific-activity positron emitter with a half-life (t_{1/2}) of 4.18 days. The biophysical properties of modified duplex were assessed using a C16-siRNA iodinated with non-radioactive iodine. In particular, Log D was determined via RP-HPLC, and the effects of hexylamine modification and iodobenzoate conjugation on siRNA hydrophobicity were shown to be minimal. Additionally the functional activity of the modified duplex was assessed using an in vitro cell-based assay. Primary mouse hepatocytes (10,000 cells per well) were transfected with an adeno-associated viral (AAV) viral vector expressing human APP (1×10\hat{ }5 virions/well) for 24 hours, followed by media replacement and transfection with non-iodinated C16-siRNA, iodinated C16-siRNA (non-radioactive), or N-acetyl-galactosamine-conjugated siRNA (GalNAc) conjugated, non-iodinated siRNA, at concentrations of 0, 0.04, 0.16, 0.62, 2.5 and 10 nM. The three siRNAs were indistinguishable in terms of their ability to interfere with APP mRNA expression (FIG. 13) demonstrating that they are functionally equivalent, and iodination has no effect on mRNA interference activity.

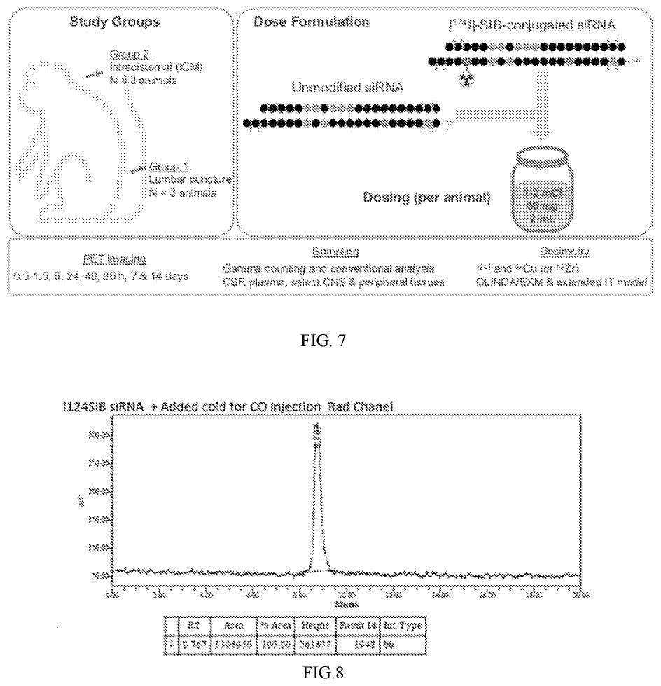

To assess the CNS distribution of radiolabeled siRNAs and determine the effect of siRNA following IT dose (up to 336 hours post-dose), a study design including two NHP study groups was formed. In Group 1, three animals were injected with radiolabeled siRNAs via ICM, while in Group 2, three animals were injected with radiolabeled siRNAs via lumbar puncture, as shown in FIG. 7. Unmodified siRNA was combined with ¹²⁴I-SIB-conjugated siRNA in a dose formulation that included about 60 mg of siRNA in a 2 ml volume with a specific activity of about 1-2 mCi (see FIG. 7). PET imaging occurred at the following timepoints: 0.5-1.5 hours, 6 hours, 24 hours, 48 hours, 96 hours, 7 days, and 14 days. The sampling was conducted via gamma counting and conventional analysis for CSF, plasma, and selected CNS and peripheral tissues. Dosimetry was conducted for ¹²⁴I and ⁶⁴Cu (or ⁸⁹Zr) by OLINDA/EXM and an extended IT model (see FIG. 7).

The kinetics and extent of brain uptake of siRNA following IT dose (up to 336 hours post-dose) was also determined, and lumbar delivery was also compared to cisterna magna delivery. The resolution of the PET method for utility in quantitative brain distribution analysis (SUV measurements) was evaluated. The above-described study dosing in summarized in Table 9.

TABLE 9
NHP CNS Biodistribution Study Design

| | Animal | | | |
|---|---|---|---|---|
| Group | No. | Route | Volume | Dose |
| 1 | 3 | IT- | 2 mL | 60 mg |
| | | Lumbar | | C16-siRNA, |
| | | | | 1-2 mCi |
| 2 | 3 | IT- | 2 mL | 60 mg |
| | | Cisterna Magna | | C16-siRNA, |
| | | | | 1-2 mCi |

Sample collection for gamma counting and conventional analysis occurred as follows. Briefly, CSF was collected on imaging days, starting at 24 hours post-dose. Plasma was collected at 5 minutes, 30 minutes, 1 hour, 2 hours, and 4 hours, in addition to imaging time points. Terminal collection took place at 14 days post-dose in the following brain and spine sub-regions: prefrontal cortex, temporal cortex, hippocampus, striatum, brainstem, hypothalamus, thalamus, cerebellum, and meninges, and the cervical, thoracic, and lumbar spinal cord. Collections also took place for the liver, heart, kidneys, and skin. Human dosimetry estimates for each administration route included ¹²⁴I and ⁶⁴Cu as assessed via OLINDA/EXM and extended IT model.

¹²⁴I-SIB-conjugated siRNA was radiolabeled for the NHP PET studies described herein. The siRNA formulation for lumbar puncture had a total yield of about 7 mCi of purified material with a specific activity/dose of about 1.7 mCi. The siRNA formulation for ICM had a total yield of about 26 mCi of ¹²⁴I and about 19.5 mCi of ¹²⁴I-SIB-conjugated siRNA. As shown in the graph in FIG. 8, the labeled ¹²⁴I-SIB-conjugated siRNA had a specific activity of about 8.2 mCi, where the total amount (labeled (hot) and unlabeled (cold) probe) was 240 mg in a volume of 8 mL. The final dose volume was 2 mL.

Pilot labeling experiments including ¹²⁵I were conducted, and radiolabeling and aCSF stability data are summarized in Table 10 and Table 11, respectively. The ¹²⁵I-siRNA stability_{aCSF} was shown to be at least 14 days.

TABLE 10
Summary of ¹²⁵I-SIB-siRNA Radiolabeling
¹²⁵I-SIB-siRNA Production

| Conjugation Buffer | Phosphate Buffer | Borate Buffer | Time |
|---|---|---|---|
| Starting | 2300 | 2110 | 10:30 (EOS) |
| Activity (μCi) | | | |
| Final Product | 1209 | 320 | 13:45 (SOS) |
| Activity (μCi) | | | |
| Radiochemical | 52 | 14 | 13:45 (SOS) |
| Yield % | | | |
| Radiochemical | ≥99 | ≥99 | |
| Purity | | | |
| (Radio-HPLC) % | | | |
| Final Product | 550 | 600 | |
| Volume (μL) | | | |
| Final Product | 2198 | 533 | |
| Concentration | | | |
| (μCi/mL) | | | |
| Specific | 2.2 | 0.5 | |
| Activity (μCi/ug) | | | |

(SOS): start of synthesis
(EOS): end of synthesis

TABLE 11
Summary of ¹²⁵I-SIB-siRNA Stability Assessment in aCSF at 37° C.

| | | 1 | 4 | 2 | 7 | 7 | 1 |
|---|---|---|---|---|---|---|---|
| Time | EOS | Hour | Hours | 4 Hours | 2 Hours | Days | 4 Days |
| Stability | ≥99% | ≥99% | ≥99% | ≥99% | ≥98% | ≥95% | ≥94% |
| (%) | | | | | | | |
| in | | | | | | | |
| Phosphate | | | | | | | |
| Buffer | | | | | | | |
| Stability | ≥99% | ≥99% | ≥99% | ≥98% | ≥98% | ≥98% | ≥97% |
| (%) | | | | | | | |
| in Borate | | | | | | | |
| Buffer | | | | | | | |

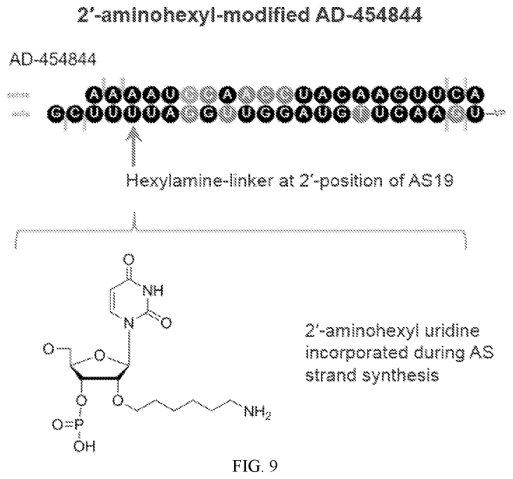

Duplex AD-454844 was radiolabeled with ¹²⁴I. Briefly, 2′-aminohexyl uridine was incorporated into the duplex during antisense strand synthesis as shown in FIG. 9. SIB-labeling then occurred during a two-step process involving an initial iodination of a tin-butyl precursor to make ¹²⁴I-SIB, followed by conjugation of ¹²⁴I-SIB to 2′-aminohexyl-AD-454844 as shown previously in FIG. 6B.

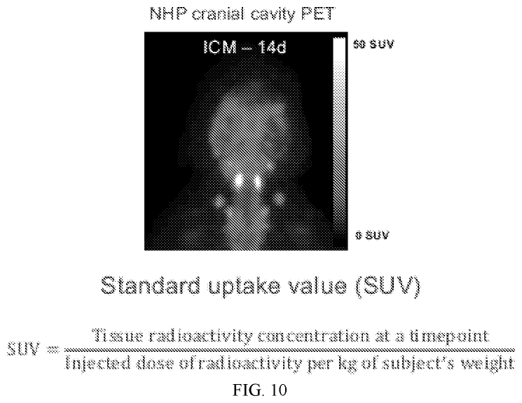

IT and ICM achieved delivery of the siRNA to the brain through the primary CSF flow routes within 30 min. ¹²⁴I-SIB-conjugated siRNA was shown to clear the CSF quickly, presumably due to systemic drainage. Significant peripheral tissue distribution of ¹²⁴I-SIB-conjugated siRNA was observed, with the liver having the highest retention. This observation was reminiscent of similar rodent studies. Interestingly, there were marked differences in uptake within the cranial cavity between the IT and ICM cohorts. FIG. 10 shows uptake of ¹²⁴I-SIB-conjugated siRNA in the cranial cavity as assayed by PET. Imaging confirmed the long retention of the siRNA within the CNS and periphery, corroborating the findings of unlabeled (e.g., “cold”) studies, and underscoring the stability of the radiolabel siRNA and the fidelity of the observed distribution pattern.

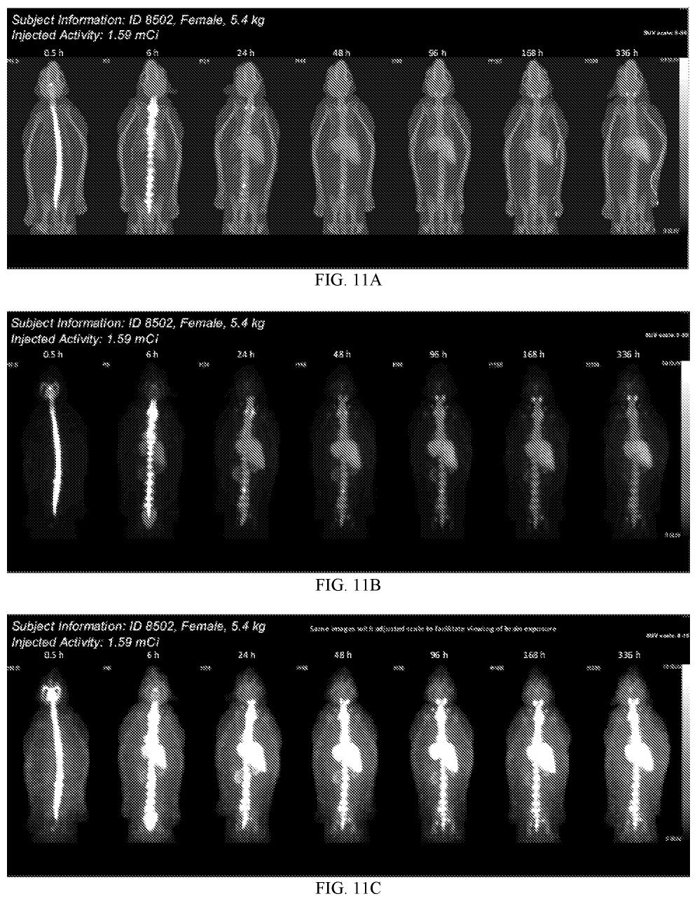

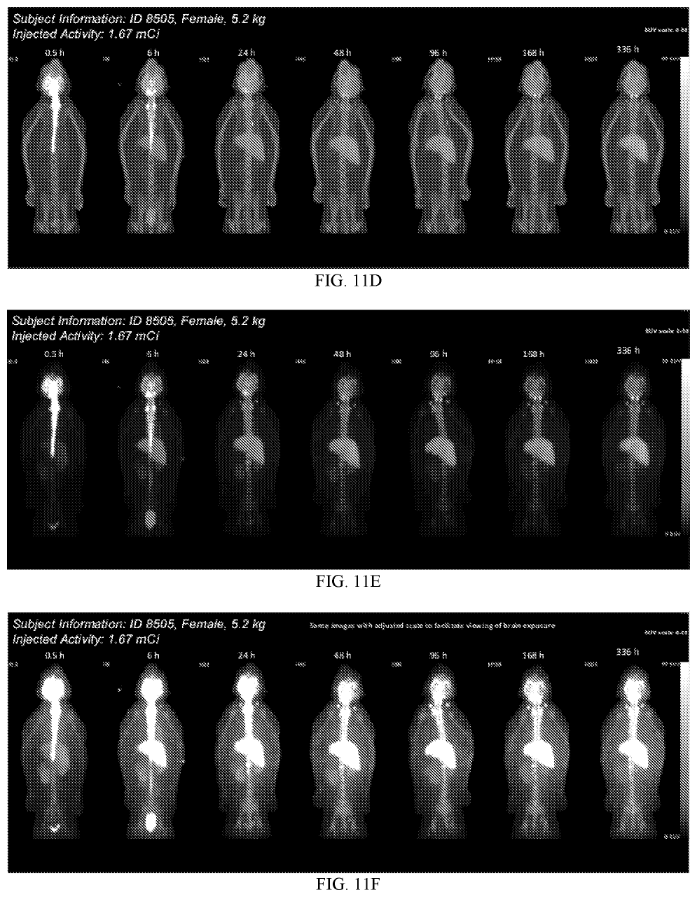

Representative images for an IT-dosed cohort of NHP animals are shown in FIG. 11A through FIG. 11F. Data from one subject are shown in FIG. 11A through FIG. 11C, and data from another subject are shown in FIG. 11D through FIG. 10F. In all of these figures, co-registered PET/CT images serve to facilitate anatomical orientation. As shown in FIG. 11A and FIG. 11B, PET reveals rapid movement of ¹²⁴I-SIB-conjugated siRNA through CSF to brain (<1 h) followed by drainage to systemic circulation. FIG. 11C shows that PET at higher-sensitivity scaling shows wide distribution across the body, yet displays long retention within the CNS. Similar to the subject data shown in FIG. 11A through FIG. 11C, PET data for the second subject is shown in FIG. 11D through FIG. 11F and reveals rapid movement through CSF to brain and cervical/thoracic spine (<1 h), followed by drainage. FIG. 11F shows that PET at higher-sensitivity scaling shows significant CNS retention and peripheral distribution.

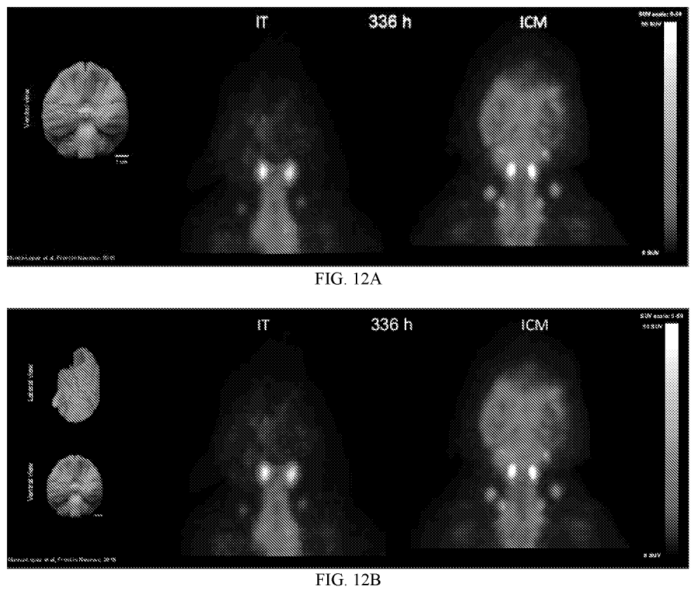

FIG. 12A and FIG. 12B show representative NHP brain images two weeks after dosing. This data shows that ICM achieves better uptake within the cranial cavity than IT dosing. Differences in parenchymal exposure (especially deep brain) await more detailed analysis.

### Table 15: Abbreviations of Nucleotide Monomers Used in Nucleic Acid Sequence Representation.

It will be understood that these monomers, when present in an oligonucleotide, are mutually linked by 5′-3′-phosphodiester bonds; and it is understood that when the nucleotide contains a 2′-fluoro modification, then the fluoro replaces the hydroxy at that position in the parent nucleotide (i.e., it is a 2′-deoxy-2′-fluoronucleotide).

The “mRNA” sequences of the Informal Sequence Listing and certain of the “mRNA target” sequences listed herein may be noted as reciting thymine (T) residues rather than uracil (U) residues. As is apparent to one of ordinary skill in the art, such sequences reciting “T” residues rather than “U” residues can be derived from NCBI accession records that list, as “mRNA” sequences, the DNA sequences (not RNA sequences) that directly correspond to mRNA sequences. Such DNA sequences that directly correspond to mRNA sequences technically constitute the DNA sequence that is the complement of the cDNA (complementary DNA) sequence for an indicated mRNA. Thus, while the mRNA target sequence does, in fact, actually include uracil (U) rather than thymine (T), the NCBI record-derived “mRNA” sequence includes thymine (T) residues rather than uracil (U) residues.

TABLE 15

| Abbre- | |
|---|---|
| viation | Nucleotide(s) |
| A | Adenosine-3′-phosphate |
| Aam | 2′-O-(N-methylacetamide)adenosine-3′-phosphate |
| Aams | 2′-O-(N-methylacetamide)adenosine-3′-phosphorothioate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| Agn | Adenosine-glycol nucleic acid (GNA), S-isomer |
| Ahd | 2′-O-hexadecyl adenosine-3′-phosphate |
| Ahds | 2′-O-hexadecyl-adenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cgn | Cytidine-glycol nucleic acid (GNA), S-isomer |
| Chd | 2′-O-hexadecyl cytidine-3′-phosphate |
| Chds | 2′-O-hexadecyl-cytidine-3′-phosphorothioate |
| Cs | cytidine-3′-phosphorothioate |
| G | guanosine-3′-phosphate |
| Gam | 2′-O-(N-methylacetamide)guanosine-3′-phosphate |
| Gams | 2′-O-(N-methylacetamide)guanosine-3′-phosphorothioate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Ggn | Guanosine-glycol nucleic acid (GNA), S-isomer |
| Ghd | 2′-O-hexadecyl guanosine-3′-phosphate |
| Ghds | 2′-O-hexadecyl-guanosine-3′-phosphorothioate |
| Gs | guanosine-3′-phosphorothioate |
| T | 5′-methyluridine-3′-phosphate |
| Tam | 2′-O-(N-methylacetamide)thymidine-3′-phosphate |
| Tams | 2′-O-(N-methylacetamide)thymidine-3′-phosphorothioate |
| Tf | 2′-fluoro-5-methyluridine-3′-phosphate |
| Tfs | 2′-fluoro-5-methyluridine-3′-phosphorothioate |
| Tgn | Thymidine-glycol nucleic acid (GNA), S-Isomer |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Uhd | 2′-O-hexadecyl uridine-3′-phosphate |
| Uhds | 2′-O-hexadecyl-uridine-3′-phosphorothioate |
| Us | uridine-3′-phosphorothioate |
| N | any nucleotide (G, A, C, T or U), modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| (C2p) | cytidine-2′-phosphate |
| (G2p) | guanosine-2′-phosphate |
| (U2p) | uridine-2′-phosphate |
| (A2p) | adenosine-2′-phosphate |
| L96 | N-[tris(GalNAc)-amido-dodecanoyl)]-4-hydroxyprolinol [Hyp-(GalNAc- |
| | alkyl)3] |
| | 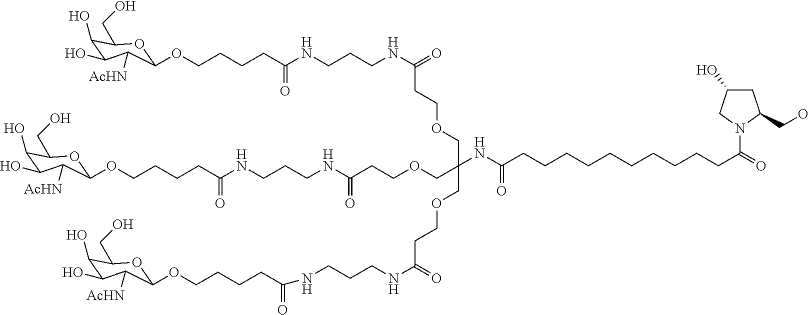 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| Y34 | 2-hydroxymethyl-tetrahydrofurane-4-methoxy-3-phosphate (abasic 2′- |
| | OMe furanose) |
| Y44 | inverted abasic DNA (2-hydroxymethyl-tetrahydrofurane-5-phosphate) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| Aeo | 2′-O-methoxyethyladenosine-3′-phosphate |
| Aeos | 2′-O-methoxyethyladenosine-3′-phosphorothioate |
| Geo | 2′-O-methoxyethylguanosine-3′-phosphate |
| Geos | 2′-O-methoxyethylguanosine-3′-phosphorothioate |
| Teo | 2′-O-methoxyethyl-5-methyluridine-3′-phosphate |
| Teos | 2′-O-methoxyethyl-5-methyluridine-3′-phosphorothioate |
| m5Ceo | 2′-O-methoxyethyl-5-methylcytidine-3′-phosphate |
| m5Ceos | 2′-O-methoxyethyl-5-methylcytidine-3′-phosphorothioate |
| A3m | 3′-O-methyladenosine-2′-phosphate |
| A3mx | 3′-O-methyl-xylofuranosyladenosine-2′-phosphate |
| G3m | 3′-O-methylguanosine-2′-phosphate |
| G3mx | 3′-O-methyl-xylofuranosylguanosine-2′-phosphate |
| C3m | 3′-O-methylcytidine-2′-phosphate |
| C3mx | 3′-O-methyl-xylofuranosylcytidine-2′-phosphate |
| U3m | 3′-O-methyluridine-2′-phosphate |
| U3mx | 3′-O-methyl-xylofuranosyluridine-2′-phosphate |
| m5Cam | 2′-O-(N-methylacetamide)-5-methylcytidine-3′-phosphate |
| m5Cams | 2′-O-(N-methylacetamide)-5-methylcytidine-3′-phosphorothioate |
| P | Phosphate |
| pshe | Hydroxyethylphosphorothioate |
| VP | Vinyl-phosphonate |
| (Aah) | 2′-O-(6-aminohexyl)-adenosine-3′-phosphate |
| (Gah) | 2′-O-(6-aminohexyl)-guanosine-3′-phosphate |
| (Cah) | 2′-O-(6-aminohexyl)-cytidine-3′-phosphate |
| (Uah) | 2′-O-(6-aminohexyl)-uridine-3′-phosphate |
| Y196 | 2′-O-(6-aminohexyl-metaiodobenzoyl)-uridine-3′-phosphate (Uah with |
| | iodo-benzoyl) |

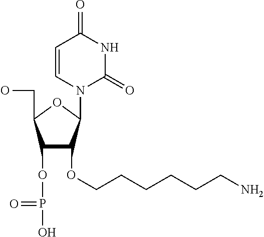

(Uah)
Chemical Formula: C₁₅H₂₄N₃O₈P
Exact Mass: 405.13
Molecular Weight: 405.34 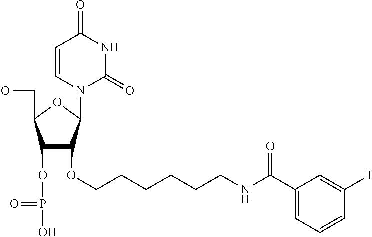
Y196
Chemical Formula: C₂₂H₂₇IN₃O₉P
Exact Mass: 635.053
Molecular Weight: 635.348

### Example 6: Biodistribution of Radiolabeled siRNAs in CNS of Non-Human Primates

As described above in Example 5, PET was used to assess the distribution of CNS siRNAs in NHPs. Iodinated C16-siRNAs were generated for use as PET probes. Briefly, a covalently linked radiolabel (e.g., Iodine-124 (¹²⁴I)) was introduced at an internal, metabolically stable position as shown in FIG. 6B and FIG. 7 (top right panel). ¹²⁴I is a high energy, high specific-activity positron emitter with a half-life (t½) of 4.18 days.

To assess the CNS distribution of radiolabeled siRNAs and determine the effect of siRNA following IT dose (up to 336 hours post-dose), a study design including two NHP study groups was formed. In Group 1, three animals were injected with radiolabeled siRNAs via IT lumbar puncture. In Group 2, three animals were injected with radiolabeled siRNAs via IT cisterna magna as shown in Table 16.

TABLE 16
NHP CNS Biodistribution Study Design

| | Animal | | | |
|---|---|---|---|---|
| Group | No. | Route | Volume | Dose |
| 1 | 3 | IT via | 2 mL | 60 mg |
| | | Lumbar Puncture | | C16-siRNA, |
| | | | | 1-2 mCi |
| 2 | 3 | IT via | 2 mL | 60 mg |
| | | Cisterna Magna | | C16-siRNA, |
| | | | | 1-2 mCi |

Unmodified siRNA was combined with ¹²⁴I-SIB-conjugated siRNA in a dose formulation that included about 60 mg of siRNA in a 2 ml volume with a specific activity of about 1-2 mCi (see FIG. 7). PET imaging occurred at the following timepoints: 0.5-1.5 hours (dynamic scan), 6 hours, 24 hours, 48 hours, 96 hours, 7 days, and 14 days. Ex vivo sampling was conducted via gamma counting and conventional analysis for CSF, plasma, and selected CNS and peripheral tissues. CSF was collected on imaging days, starting at 24 hours post-dose. In addition to the imaging time points, plasma was collected at 5 minutes, 30 minutes, 1 hour, 2 hours, and 4 hours. Terminal collection took place at 14 days post-dose in the following brain and spine sub-regions: prefrontal cortex, temporal cortex, hippocampus, striatum, brainstem, hypothalamus, thalamus, cerebellum, and meninges, and the cervical, thoracic, and lumbar spinal cord. Collections also took place for the liver, heart, kidneys, and skin.

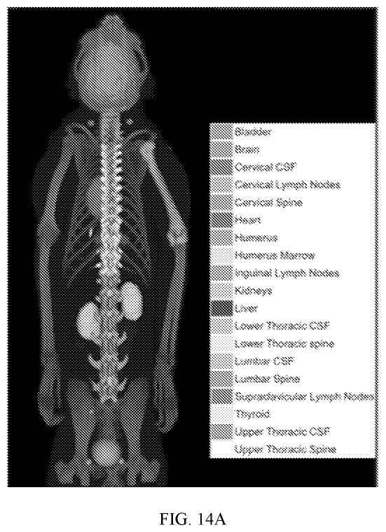

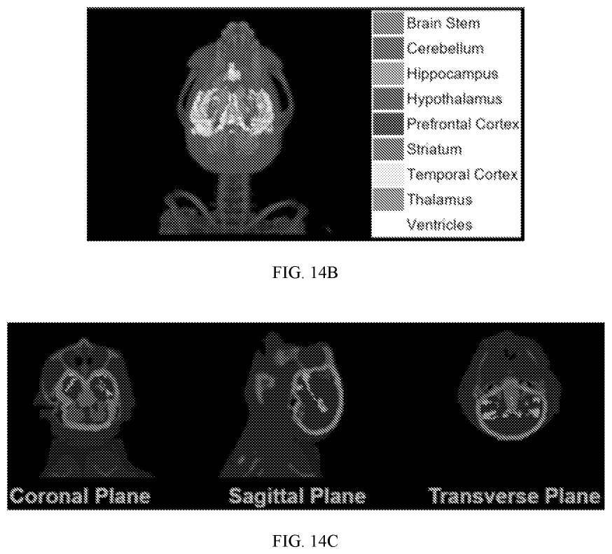

PET/CT image processing was conducted as follows. PET and CT images were co-registered and resampled to uniform voxel size (0.6 mm)³ using VivoQuant software (Invicro). FIG. 14A shows the systemic regions that were segmented for image analysis, including: bladder, brain, cervical CSF, cervical lymph nodes, cervical spine, heart, humerus, humerus marrow, inguinal lymph nodes, kidneys, liver, lower thoracic CSF, lower thoracic spine, lumbar CSF, lumbar spine, supraclavicular lymph nodes, thyroid, upper thoracic CSF, and upper thoracic spine. Several Regions of Interests (ROIs) were identified, which include, but are not limited to, brain, bladder, thyroid, cervical CSF, upper and lower thoracic CSF, lumbar CSF, cervical spine, upper and lower thoracic spine, lumbar spine, cervical lymph nodes, inguinal lymph nodes, liver, supraclavicular lymph nodes, humerus, humerus marrow, heart, and kidney, which are described in more detail below.

Brain ROIs included all regions of the brain and part of the meninges (e.g., inner meninges such as the pia mater).

Bladder and thyroid ROIs were segmented to encompass all activity within the tissues. In cases where no activity was evident in the region, the ROI was defined by placing one (e.g., gallbladder and bladder) or two (e.g., thyroid) spherical volume(s) in the corresponding anatomical location.

Cervical CSF, upper and lower thoracic CSF, and lumbar CSF ROIs were generated to encompass all activity within the spinal canal (e.g., spinal cord and CSF). In cases where no activity was evident in the region, the ROI was defined based upon the corresponding CT image. Cervical spine, upper and lower thoracic spine, and lumbar spine ROIs were generated by a connected thresholding method on CT image to encompass the corresponding spinal vertebrae.

Cervical lymph nodes, inguinal lymph nodes, liver and supraclavicular lymph nodes ROIs were defined by placing two spheres in their corresponding anatomical locations based on CT and PET uptake where present.

The humerus ROI was defined by CT-based thresholding on the right humerus bone. The marrow ROI was defined as the soft tissue inside the humerus bone. This region was defined manually post-humerus ROI generation.

Heart and kidney ROIs consisted of whole organ phantoms placed in their corresponding anatomical locations based on CT and PET uptake where present.

As shown in FIG. 14B, nine brain sub-regions (e.g., brain stem, cerebellum, hippocampus, hypothalamus, prefrontal cortex, striatum, temporal cortex, thalamus, and ventricles) were generated by registering Invicro's NHP brain atlas to the in vivo brain data based on the CT image (FIG. 14B). Whole brain ROI was generated by combining all the subregions into one single region.

As shown in FIG. 14C, meninges ROI consisted of the inner and outer meninges regions. The outer meninges was created by eroding the edges of the whole brain ROI. The inner meninges region was created by thresholding on the cerebellum and brain stem. The threshold limit was defined as 7-35% of the whole brain uptake. The inner and outer meninges were combined to create the full meninges region. The ROI was then manually revised to exclude any regions that were not part of the meninges.

Quantification within the images was determined as follows. ¹²⁴I-siRNA radioactivity within each ROI was quantified across all time points as a percent of injected dose (% ID), percent of injected dose per unit mass of the ROI (% ID/g) and standardized uptake value (SUV).

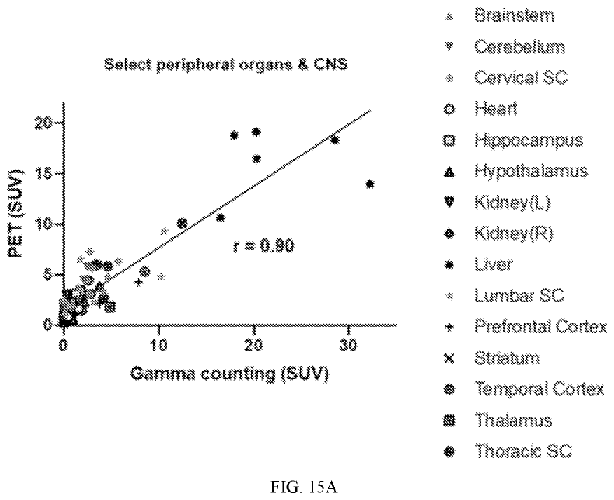

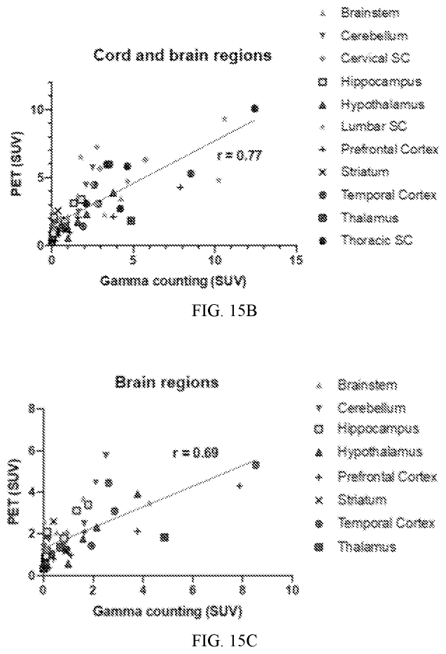

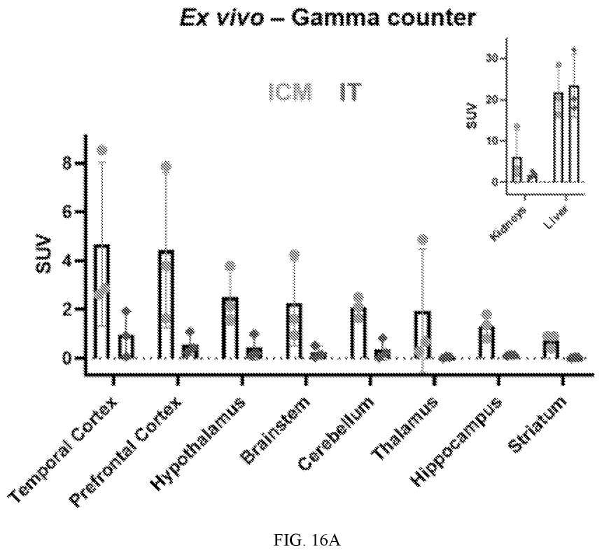

In vivo vs. ex vivo analysis was conducted by correlating tissue quantification with image analysis. As shown in FIG. 15A, gamma counting (SUV) was plotted vs. PET (SUV) in selected peripheral organs and the CNS (e.g., brain stem, cerebellum, cervical spinal cord, heart, hippocampus, hypothalamus, left kidney, right kidney, liver, lumbar spinal cord, prefrontal cortex, striatum, temporal cortex, thalamus, and thoracic spinal cord) and showed a r value of 0.90.

FIG. 15B shows the results of a similar analysis in the cord and brain regions (e.g., brain stem, cerebellum, cervical spinal cord, hippocampus, hypothalamus, lumbar spinal cord, prefrontal cortex, striatum, temporal cortex, thalamus, and thoracic spinal cord) and showed a r value of 0.77.

FIG. 15C shows the results of a similar analysis in the brain regions (e.g., brain stem, cerebellum, hippocampus, hypothalamus, prefrontal cortex, striatum, temporal cortex, and thalamus) and showed an r value of 0.69. It is noted that small differences between data points for individual regions may have occurred due to tissue rinsing or signal attenuation. In general, the data presented in FIGS. 15A-15C shows a good correlation between imaging and tissue quantification.

In summary, SPECT/CT and PET/CT analyses provided a wealth of spatial and temporal information of tested siRNA conjugates in the CNS, and revealed apparent similarities in clearance and retention between rodents and NHP, with liver having the highest exposure. Parallels between rat and NHP distribution increase confidence in the translation of the results of the described studies, both above and below, to humans. The data also indicate that ¹¹¹Indium and ¹²⁴Iodine can serve as tracers for future pre-clinical platform studies.

### Example 7: ICM Dosing Results in Improved Uptake of siRNAs in CNS of NHPs

To assess the efficacy of ICM delivery, ex vivo and in vivo analyses in NHPs were used to compare siRNA uptake in CNS tissues (e.g., temporal cortex, prefrontal cortex, hypothalamus, brain stem, cerebellum, thalamus, hippocampus, striatum, and ventricle) by both ICM delivery and IT delivery. The uptake of siRNA into kidneys and liver was also assessed and used as a non-CNS tissue control.

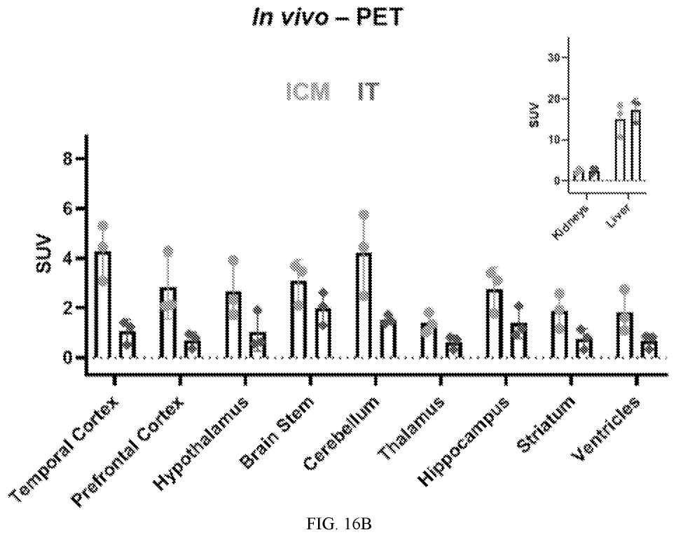

FIG. 16A is a bar graph of an ex vivo analysis conducted 14 days post-delivery that shows gamma counting (SUV) for eight CNS tissues (temporal cortex, prefrontal cortex, hypothalamus, brain stem, cerebellum, thalamus, hippocampus, and striatum) following either ICM (circles) or IT (diamonds) delivery. The ex vivo analysis demonstrated that ICM delivery yielded a significant increase of siRNA uptake in the assayed CNS tissues relative to IT delivery. In particular, ICM siRNA delivery produced a 5-8× increase in siRNA uptake into both the temporal and prefrontal cortex relative to IT delivery. Additionally, ICM siRNA delivery also produced an approximately 6× increase in siRNA uptake into the cells of the hypothalamus, brain stem, cerebellum, thalamus, hippocampus, and striatum, relative to IT delivery. The inset of FIG. 16A shows gamma counting (SUV) in the kidneys and liver, as a non-CNS control, and shows that both ICM and IT delivery resulted in similar levels of uptake into the liver. ICM delivery appeared to increase kidney uptake by approximately 3-fold over IT, but the % CV on the ICM kidney mean was >100%, so this estimate may be unreliable.

FIG. 16B is a bar graph of an in vivo analysis conducted 14 days post-delivery that shows PET (SUV) results for nine CNS tissues (temporal cortex, prefrontal cortex, hypothalamus, brain stem, cerebellum, thalamus, hippocampus, striatum, and ventricles) following either ICM (circles) or IT (diamonds) delivery. The in vivo analysis demonstrated that ICM delivery yielded a significant increase of siRNA uptake in the assayed CNS tissues, relative to IT delivery. In particular, ICM siRNA delivery produced a 2-4× increase in siRNA uptake into the temporal cortex, prefrontal cortex, hypothalamus, brain stem, cerebellum, thalamus, hippocampus, striatum, and ventricles, relative to IT delivery. The inset depicts analysis of PET (SUV) in the kidneys and liver as a non-CNS control, and shows that both ICM and IT delivery resulted in similar levels of uptake into both the liver and the kidneys.

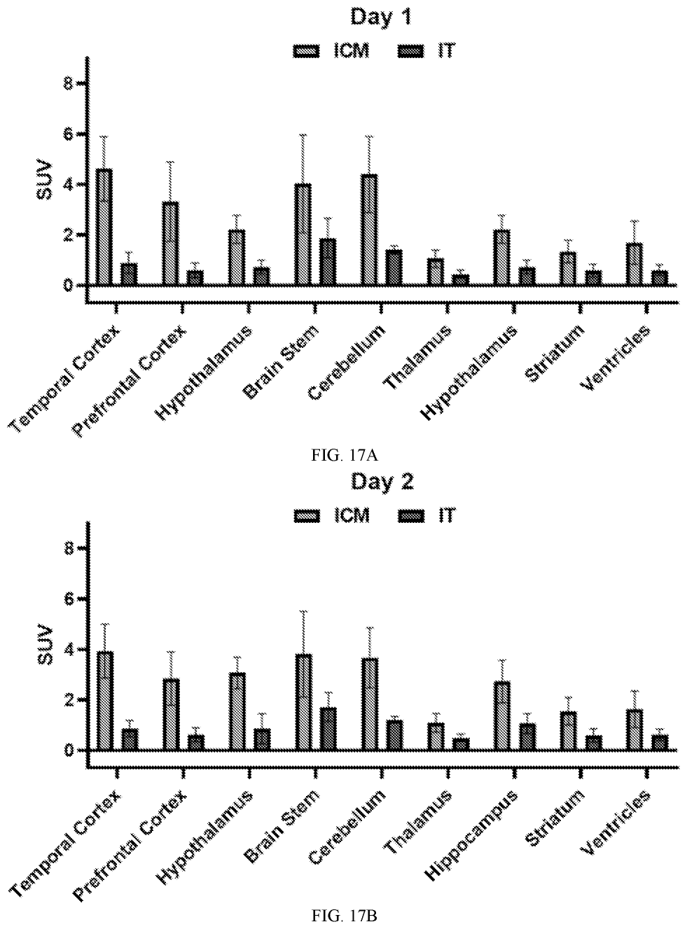

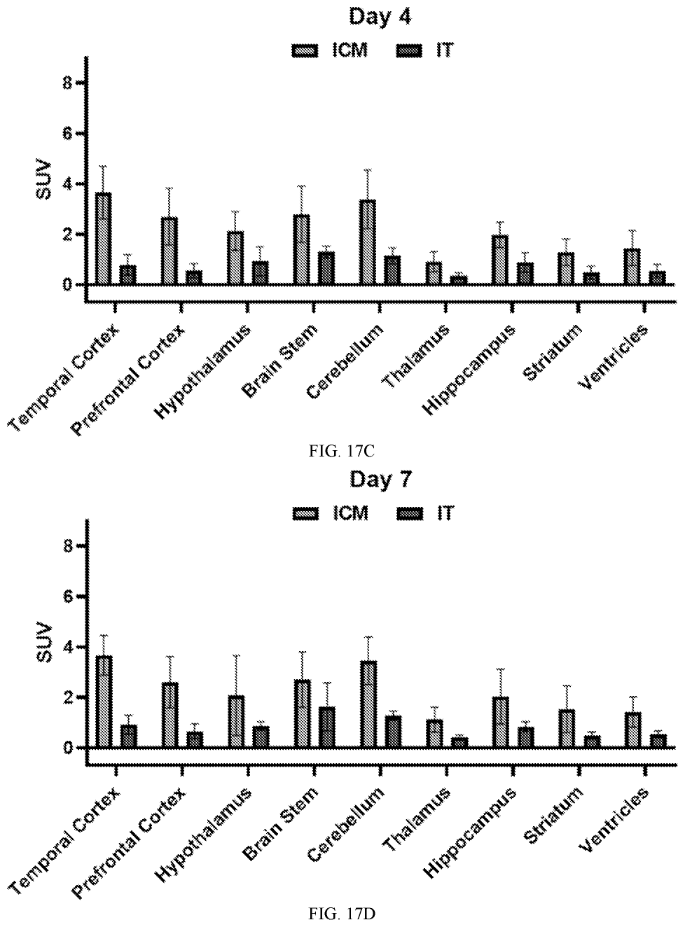

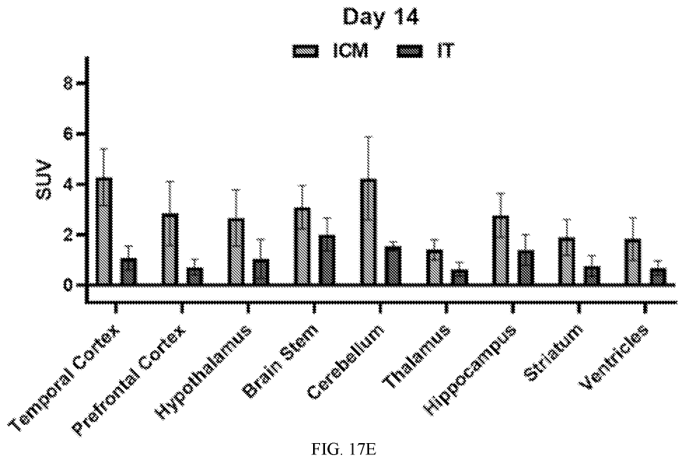

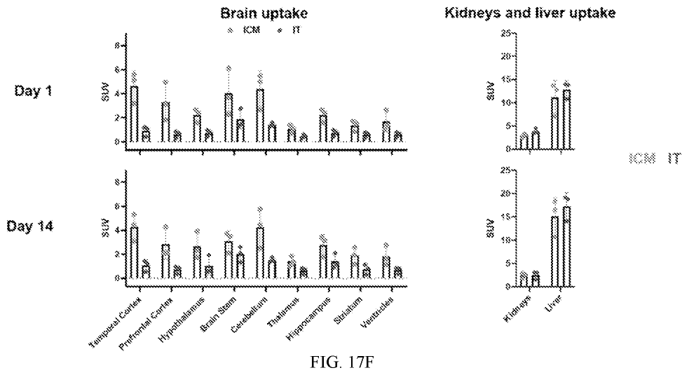

A time course analysis of the in vivo PET (SUV) experiments demonstrated that ICM delivery yielded a significant increase of siRNA uptake in the assayed CNS tissues relative to IT delivery, from Day 1 through the last study timepoint on Day 14. For example, FIG. 17A, FIG. 17B, FIG. 17C, FIG. 17D, and FIG. 17E show PET (SUV) data for Day 1, Day 2, Day 4, Day 7, and Day 14, respectively. This time lapse analysis showed that ICM delivery significantly outperformed IT delivery in all CNS tissues analyzed. As shown in FIG. 17F, comparison of siRNA uptake at the initial timepoint of Day 1 relative to the final timepoint of Day 14 showed sustained siRNA retention in all assayed CNS tissues, with ICM delivery outperforming IT delivery in all CNS tissues examined. Notably, this includes deep brain regions such as the striatum, hippocampus, and thalamus, suggesting that ICM delivery may help in reaching less accessible parts of the brain. In particular, robust siRNA delivery by ICM was observed in the temporal cortex, the prefrontal cortex, the cerebellum, and the brain stem.

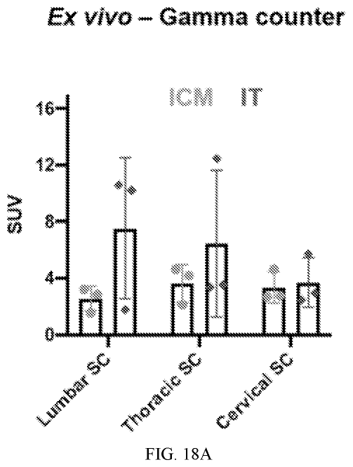

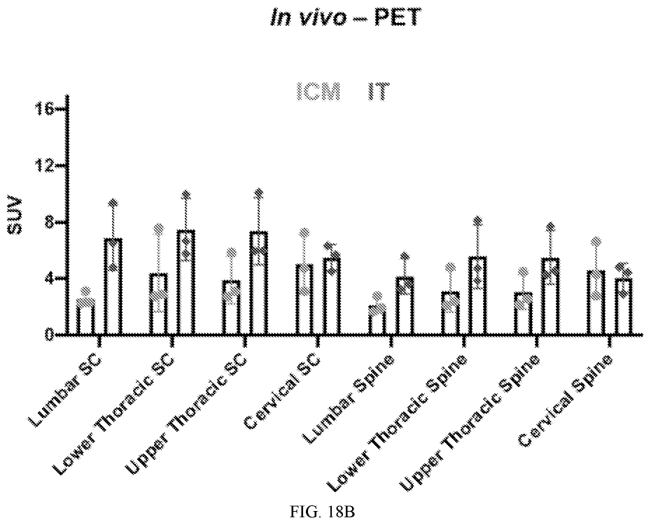

In contrast, IT dosing exhibited better lumbar spinal cord (SC) uptake than ICM at Day 14. For example, FIG. 18A shows the results of an ex vivo experiment in which gamma counting (SUV) was assessed in lumbar SC, thoracic SC, and cervical SC tissues. The lumbar SC and thoracic SC uptake appeared to be higher, on average, following IT dosing relative to ICM, although the % CV values were too large to determine explicit fold-change values. However, siRNA dosing at Day 14 was approximately the same in cervical SC tissues whether delivered by IT or ICM. The differences were clearer by in vivo PET analysis, where 2-3-fold higher uptake was observed in lumbar SC and spine by IT dosing, and 2-fold higher in the lower and upper thoracic SC and spine (see FIG. 18B).

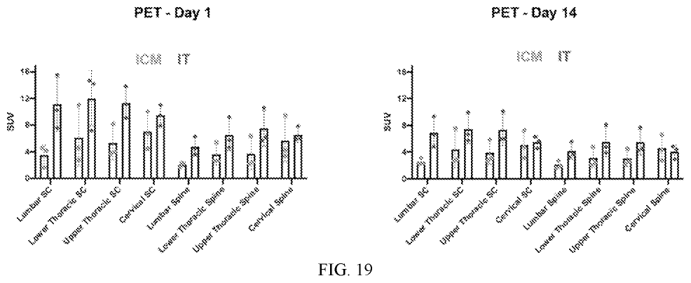

IT dosing produced better siRNA uptake in the spinal cord across the neuraxis. For example, FIG. 19 shows comparative bar graphs of an in vivo PET analysis showing better uptake by IT delivery in the lumbar SC and spine, lower thoracic SC and spine, and upper thoracic SC and spine at Day 1 and Day 14.

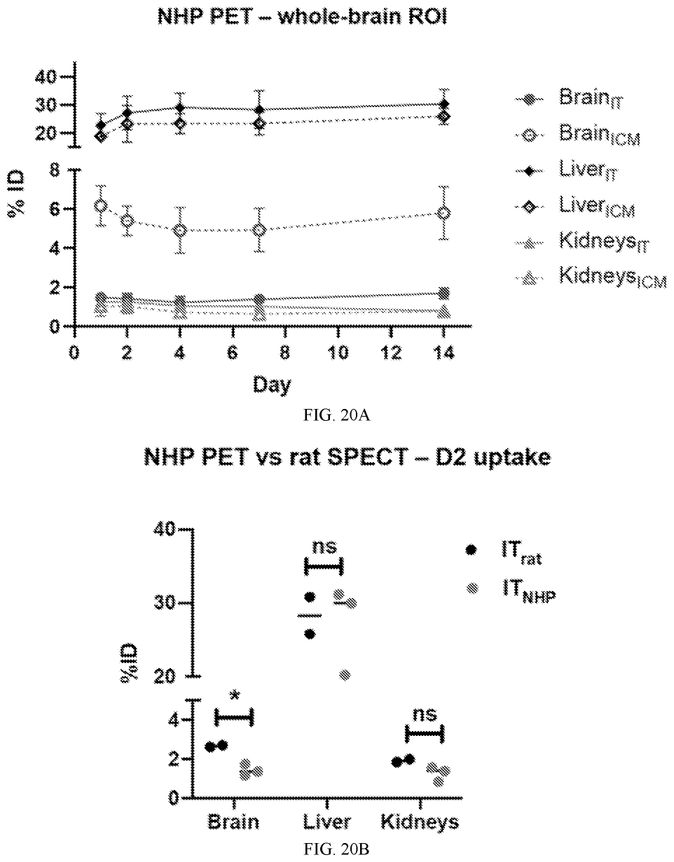

A longitudinal overview of the uptake experiments indicated that ICM delivery outperformed IT delivery in brain retention as assessed across whole-brain ROI. For example, FIG. 20A is a graph depicting a longitudinal overview of ICM delivery and IT delivery of siRNA in the brain, liver, and kidneys of NHPs, with PET data collected at Day 1, Day 2, Day 4, Day 7, and Day 14 shown on the x-axis and % of injected dose (% ID) shown on the y-axis. IT delivery is shown by solid circles (brain), solid diamonds (liver), and solid triangles (kidneys), while ICM delivery is shown by open circles (brain), open diamonds (liver), and open triangles (kidneys). The observed % ID in the brain ROI was significantly higher by ICM dosing, while % ID in the liver and kidneys was only slightly higher by IT dosing. FIG. 20B is a dot plot of NHP PET data vs. rat SPECT data at Day 2 post-delivery showing that IT delivery produced low levels of delivery (about 2% ID) in the brains of both rat and NHP, which is consistent with the NHP brain levels shown in FIG. 20A.

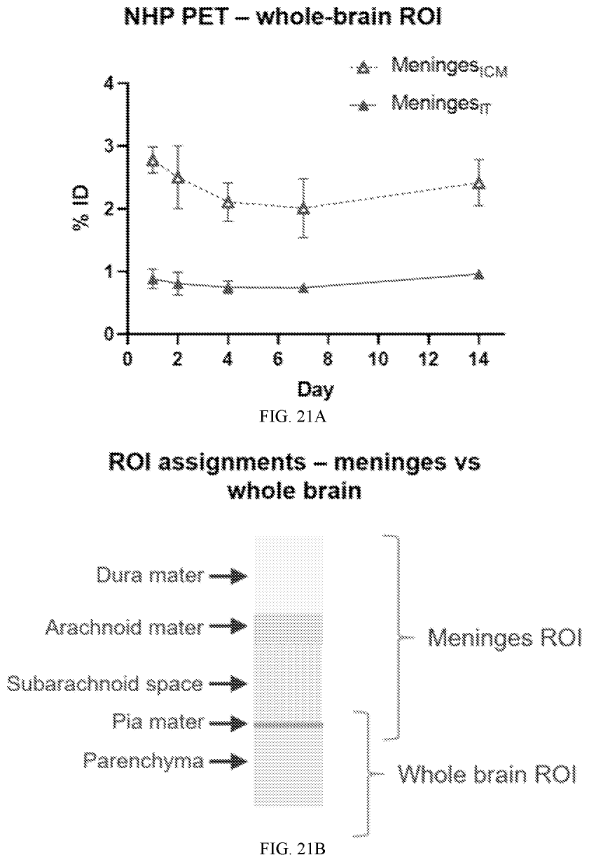

As part of the comparative analysis of IT and ICM delivery routes, siRNA uptake into the meninges was also assessed. FIG. 21A is a graph that shows the results of PET analysis of siRNA uptake into the meninges of NHP subjects as assessed by % ID (y-axis) following either ICM delivery (open triangles) or IT delivery (solid triangles) at Day 1, Day 2, Day 4, Day 7, and Day 14 (x-axis). ICM delivery resulted in a significant increase in % ID relative to IT delivery at all measured time points. It is important to note that the meninges ROI displays partial overlap with the whole brain ROI, so it is not trivial to directly compare the two ROIs. FIG. 21B is a diagram showing the ROI assignments in the meninges and the brain, including regions of partial overlap including a portion of the parenchyma, the pia mater, and a portion of the subarachnoid space (see e.g., Ringstad and Eide (2020) Cerebrospinal fluid tracer efflux to parasagittal dura in humans. Nat. Com. January 17; 11(1):354). The analyses to date indicate that whole brain ROI % ID ranges between about 1% and about 7% (including IT and ICM delivery) and meninges ROI % ID ranges between about 1% and about 3%. These results indicate that there was a significant amount of material observed in the meninges ROI relative to whole brain ROI; however, drug levels (e.g., siRNA) in meninges samples will need to be assessed via non-radioactive studies to understand more completely the drug uptake profile of the meninges ROI.

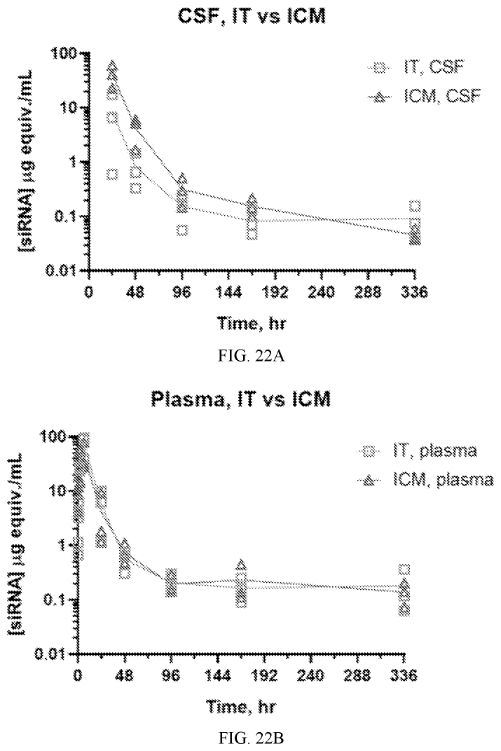

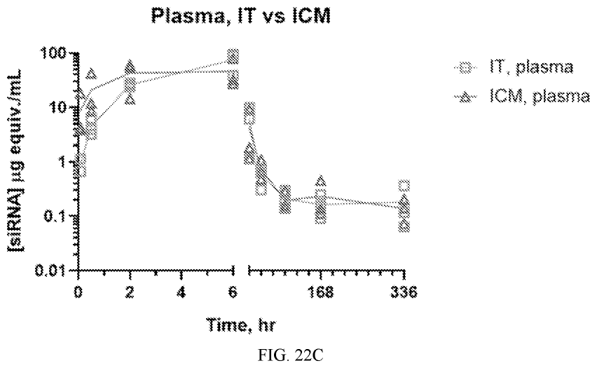

Gamma counting was used to assess siRNA present in both CSF and plasma. FIG. 22A is a graph showing the concentration of siRNA in μg equivalents/ml (y-axis) in CSF as assessed by gamma counting following either IT delivery (open squares) or ICM delivery (open triangles) at 24, 48, 96, 168, or 336 hours (x-axis). FIG. 22B is a graph showing the concentration of siRNA in μg equivalents/ml (y-axis) in plasma as assessed by gamma counting following either IT delivery (open squares) or ICM delivery (open triangles) at 24, 48, 96, 168, or 336 hours (x-axis). FIG. 22C is a modified version of FIG. 22B showing the concentration of siRNA in μg equivalents/ml (y-axis) in plasma as assessed by gamma counting following either IT delivery (open squares) or ICM delivery (open triangles) at 24, 48, 96, 168, or 336 hours (x-axis), where the x-axis is re-scaled to highlight the plasma siRNA profile in the first 48 hours. These data showed that CSF and plasma profiles appeared to be generally consistent with prior observations in that there was a rapid initial uptake observed up to 48 hours, followed by a plateau thereafter. As shown in FIG. 22A, CSF levels were slightly higher following ICM delivery versus IT delivery (earliest timepoint is 24 h). Similarly, plasma levels were also higher following ICM delivery at less than 2 hours (see, e.g., FIG. 22C).

In summary, the above data confirmed the apparent similarities in clearance and retention between rodents and NHP, with liver having the highest exposure. The data also showed parallels between rat and monkey distribution that increase confidence that these data will useful for human translation. Importantly, ICM dosing appeared to achieve better uptake by the brain parenchyma than IT dosing. The temporal cortex and cerebellum were observed to have the highest brain tissue retention, whereas the thalamus exhibited the lowest. Meanwhile, the meninges retained a significant amount of either ICM- or IT-dosed siRNA.

1. A method of reducing the expression of a target gene in a central nervous system (CNS) tissue or cell of a subject via administration to a subarachnoid space of the subject, comprising:
contacting the tissue or cell with a double-stranded iRNA agent, wherein the agent includes
an antisense strand which comprises a region of complementarity to the target gene, optionally which comprises at least 15 contiguous nucleotides differing by no more than 3 nucleotides from any one of the antisense sequences listed in Table 2, Table 3, or Table 8;
a sense strand complementary to the antisense strand; and
one or more lipophilic moieties conjugated to one or more internal positions on at least one strand, optionally via a linker or carrier.

2. The method of claim 1, wherein the lipophilicity of the lipophilic moiety, measured by log K_{ow}, exceeds 0.

3. The method of claim 1, wherein the hydrophobicity of the double-stranded iRNA agent, measured by the unbound fraction in the plasma protein binding assay of the double-stranded iRNA agent, exceeds 0.2.

4. The method of claim 3, wherein the plasma protein binding assay is an electrophoretic mobility shift assay using human serum albumin protein.

5. The method of claim 1, wherein the lipophilic moiety contains a saturated or unsaturated C₁₆ hydrocarbon chain.

6. The method of claim 1, wherein contacting comprises:
injecting the double-stranded iRNA agent into cerebrospinal fluid (CSF) of the subarachnoid space.

7. The method of claim 6, wherein the subarachnoid space is the cisterna magna.

8. The method of claim 1, wherein the target gene is selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25.

9. The method of claim 1, wherein expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, optionally wherein expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

10. The method of claim 9, wherein expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days.

11. The method of claim 1, wherein expression of the target gene is reduced by about 20%, about 25%, about 30%, about 35%, about 40%, about 45%, about 50%, about 55%, about 60%, about 65%, about 70%, or about 75% within about 29 days.

12. The method of claim 1, wherein expression of the target gene is reduced by about 25% to about 35% within about 29 days.

13. The method of claim 1, wherein expression of the target gene is reduced by about 35% to about 45% within about 29 days.

14. The method of claim 1, wherein expression of the target gene is reduced by about 45% to about 55% within about 29 days.

15. The method of claim 1, wherein expression of the target gene is reduced by about 25% to about 50% within about 29 days.

16. The method of claim 1, wherein:
expression of the target gene is reduced by about 35% to about 50% within about 29 days;
expression of the target gene is reduced by about 45% to about 50% within about 29 days;
expression of the target gene is reduced by about 50% within about 29 days;
expression of the target gene is reduced by about 25% to about 50% within about 25 to about 31 days;
expression of the target gene is reduced by about 35% to about 50% within about 25 to about 31 days;
expression of the target gene is reduced by about 45% to about 50% within about 25 to about 31 days;
expression of the target gene is reduced by about 50% within about 25 to about 31 days; the double-stranded iRNA agent is detected in a cerebral cortex (optionally frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (optionally cervical, thoracic, and/or lumbar DRG), tissue or cell, optionally wherein the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, or wherein the double-stranded iRNA agent is detected within about 1 to about 5 days, about 5 to about 10 days, about 10 to about 15 days, about 15 to about 20 days, about 20 to about 25 days, or about 25 to about 30 days, optionally wherein the double-stranded iRNA agent is detected within about 25 days to about 30 days, optionally wherein the double-stranded iRNA agent is detected within about 29 days, or wherein the double-stranded iRNA agent is detected after at least 29 days, or wherein the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, or wherein the double-stranded iRNA agent is detected after about 1 to about 5 days, about 5 to about 10 days, about 10 to about 15 days, about 15 to about 20 days, about 20 to about 25 days, or about 25 to about 30 days, optionally wherein the double-stranded iRNA agent is detected after about 25 days to about 30 days, optionally wherein the double-stranded iRNA agent is detected after about 29 days;
the target gene is not HTT;
expression of the target gene is reduced by at least 96%, at least 97%, at least 98%, at least 99%, or at least 100%; and/or
the sense strand comprises at least 15 contiguous nucleotides differing by no more than 3 nucleotides from the nucleotide sequence of the sense strand nucleotide sequence of a duplex selected from the group consisting of AD-454844, AD-1395836, AD-1397045, AD-961583, AD-961584, AD-961585, and AD-961586.

17-36. (canceled)

37. A method of reducing the expression of a target gene in the cerebral cortex, hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) of a subject, comprising:
administering to a subarachnoid space of the subject a double-stranded iRNA agent that includes
an antisense strand which comprises a region of complementarity to the target gene, optionally which comprises at least 15 contiguous nucleotides differing by no more than 3 nucleotides from any one of the antisense sequences listed in Table 2, Table 3, or Table 8;
a sense strand complementary to the antisense strand; and
one or more lipophilic moieties conjugated to one or more internal positions on at least one strand, optionally via a linker or carrier.

38. The method of claim 37, wherein:
administering comprises injecting the double-stranded iRNA agent into cerebrospinal fluid (CSF) of the subarachnoid space, optionally wherein the subarachnoid space is or includes the cisterna magna, optionally wherein administering comprises injecting the double-stranded iRNA agent into the cisterna magna of the subject;
the cerebral cortex frontal, temporal, parietal, and/or occipital cortex;
the DRG is cervical, thoracic, and/or lumbar DRG;
the method reduces the expression of the target gene in the cerebral cortex (optionally frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (optionally cervical, thoracic, and/or lumbar DRG) of the subject;
target gene is selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25;
expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, optionally wherein expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days;
expression of the target gene is reduced by about 20%, about 25%, about 30%, about 35%, about 40%, about 45%, about 50%, about 55%, about 60%, about 65%, about 70%, or about 75% within about 29 days;
expression of the target gene is reduced by about 25% to about 35% within about 29 days;
expression of the target gene is reduced by about 35% to about 45% within about 29 days;
expression of the target gene is reduced by about 45% to about 55% within about 29 days;
expression of the target gene is reduced by about 25% to about 50% within about 29 days;
expression of the target gene is reduced by about 35% to about 50% within about 29 days;
expression of the target gene is reduced by about 45% to about 50% within about 29 days;
expression of the target gene is reduced by about 50% within about 29 days;
expression of the target gene is reduced by about 25% to about 50% within about 25 to about 31 days;
expression of the target gene is reduced by about 35% to about 50% within about 25 to about 31 days;
expression of the target gene is reduced by about 45% to about 50% within about 25 to about 31 days;
expression of the target gene is reduced by about 50% within about 25 to about 31 days;
the double-stranded iRNA agent is detected in cerebral cortex (optionally frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (optionally cervical, thoracic, and/or lumbar DRG) tissues or cells, optionally wherein the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, or the double-stranded iRNA agent is detected within about 1 to about 5 days, about 5 to about 10 days, about 10 to about 15 days, about 15 to about 20 days, about 20 to about 25 days, or about 25 to about 30 days, optionally wherein the double-stranded iRNA agent is detected within about 25 days to about 30 days, optionally wherein the double-stranded iRNA agent is detected within about 29 days, or the double-stranded iRNA agent is detected after at least 29 days, or the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days, or the double-stranded iRNA agent is detected after about 1 to about 5 days, about 5 to about 10 days, about 10 to about 15 days, about 15 to about 20 days, about 20 to about 25 days, or about 25 to about 30 days, optionally wherein the double-stranded iRNA agent is detected after about 25 days to about 30 days, optionally wherein the double-stranded iRNA agent is detected after about 29 days;
the target gene is not HTT;
expression of the target gene is reduced by at least 96%, at least 97%, at least 98%, at least 99%, or at least 100%;
expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days; and/or
the sense strand comprises at least 15 contiguous nucleotides differing by no more than 3 nucleotides from the nucleotide sequence of the sense strand nucleotide sequence of duplex AD-454844, AD-1395836, AD-1397045, AD-961583, AD-961584, AD-961585, or AD-961586.

39-69. (canceled)

70. A method of treating a subject having a CNS disorder, comprising:
administering to a subarachnoid space of the subject a therapeutically effective amount of a double-stranded iRNA agent, thereby treating the subject.

71. The method of claim 70, wherein:
the double-stranded iRNA agent includes an antisense strand which comprises a region of complementarity to the target gene which comprises at least 15 contiguous nucleotides differing by no more than 3 nucleotides from any one of the antisense sequences listed in Table 2 or Table 3, a sense strand complementary to the antisense strand, and one or more lipophilic moieties (optionally wherein the lipophilic moiety contains a saturated or unsaturated C₁₆ hydrocarbon chain) conjugated to one or more internal positions on at least one strand, optionally via a linker or carrier, optionally wherein the hydrophobicity of the double-stranded iRNA agent, measured by the unbound fraction in the plasma protein binding assay of the double-stranded iRNA agent, exceeds 0.2, or the lipophilicity of the lipophilic moiety, measured by log K_{ow}, exceeds 0, optionally wherein the plasma protein binding assay is an electrophoretic mobility shift assay using human serum albumin protein;
the CNS disorder is selected from the group consisting of Alzheimer's disease (AD), amyotrophic lateral sclerosis (ALS), frontotemporal dementia, Huntington, Parkinson, spinocerebellar, prion, and Lafora;
the CNS disorder is a disorder affecting the cerebral cortex (optionally frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (e.g., cervical, thoracic, and/or lumbar DRG);
administering comprises injecting the double-stranded iRNA agent into cerebrospinal fluid (CSF) of the subarachnoid space, optionally wherein the subarachnoid space is or includes the cisterna magna;
administering comprises injecting the double-stranded iRNA agent into the cisterna magna of the subject;
the method reduces the expression of the target gene in brain tissue surrounding the cisterna magna of the subject, optionally wherein expression of the target gene is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, optionally wherein expression of the target gene is reduced within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days and/or expression of the target gene is reduced by about 20%, about 25%, about 30%, about 35%, about 40%, about 45%, about 50%, about 55%, about 60%, about 65%, about 70%, or about 75% within about 29 days; and/or
the double-stranded iRNA agent is directed to a target gene selected from the group consisting of APP, ATXN2, C9orf72, TARDBP, MAPT(Tau), HTT, SNCA, FUS, ATXN3, ATXN1, SCA1, SCA7, SCA8, MeCP2, PRNP, SOD1, DMPK, GPR75, LRRK2, SARM1, and RPS25; expression of the target gene is reduced by about 25% to about 35% within about 29 days; expression of the target gene is reduced by about 35% to about 45% within about 29 days; expression of the target gene is reduced by about 45% to about 55% within about 29 days; expression of the target gene is reduced by about 25% to about 50% within about 29 days; expression of the target gene is reduced by about 35% to about 50% within about 29 days; expression of the target gene is reduced by about 45% to about 50% within about 29 days; expression of the target gene is reduced by about 50% within about 29 days; expression of the target gene is reduced by about 25% to about 50% within about 25 to about 31 days; expression of the target gene is reduced by about 35% to about 50% within about 25 to about 31 days; expression of the target gene is reduced by about 45% to about 50% within about 25 to about 31 days; and/or expression of the target gene is reduced by about 50% within about 25 to about 31 days; and/or the double-stranded iRNA agent is detected in the cerebral cortex (optionally frontal, temporal, parietal, and/or occipital cortex), hypothalamus, cerebellum, striatum, hippocampus, cerebellum, brain stem, hypothalamus, pituitary, cervical spine, lumbar spine, thoracic spine, trigeminal ganglion, caudate nucleus, pons/medulla, and/or dorsal root ganglion (DRG) (optionally cervical, thoracic, and/or lumbar DRG) tissues or cells, optionally wherein the double-stranded iRNA agent is detected within about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days; and/or the double-stranded iRNA agent is detected within about 1 to about 5 days, about 5 to about 10 days, about 10 to about 15 days, about 15 to about 20 days, about 20 to about 25 days, or about 25 to about 30 days, optionally wherein the double-stranded iRNA agent is detected within about 25 days to about 30 days, optionally wherein the double-stranded iRNA agent is detected within about 29 days; and/or the double-stranded iRNA agent is detected after at least 29 days; and/or the double-stranded iRNA agent is detected after about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days; and/or the double-stranded iRNA agent is detected after about 1 to about 5 days, about 5 to about 10 days, about 10 to about 15 days, about 15 to about 20 days, about 20 to about 25 days, or about 25 to about 30 days, optionally wherein the double-stranded iRNA agent is detected after about 25 days to about 30 days, optionally wherein the double-stranded iRNA agent is detected after about 29 days; and/or the sense strand comprises at least 15 contiguous nucleotides differing by no more than 3 nucleotides from the nucleotide sequence of the sense strand nucleotide sequence of duplex AD-454844, AD-1395836, AD-1397045, AD-961583, AD-961584, AD-961585, or AD-961586; and/or expression of the target gene is reduced by at least 96%, at least 97%, at least 98%, at least 99%, or at least 100%; and/or expression of the target gene is reduced within about 10 to about 20 days, about 20 to about 30 days, about 30 to about 40 days, about 40 to about 50 days, about 50 to about 60 days, about 60 to about 70 days, about 70 to about 80 days, about 80 to about 90 days, or about 90 to about 100 days.

72-108. (canceled)
